(19) United States
(12) Patent Application Publication
(10) Pub. No.: US 2015/0246332 A1
(43) Pub. Date: Sep. 3, 2015

(54) FOAMING AGENT FOR USE IN INDUSTRIAL PRODUCTS

(71) Applicant: Solac, LLC, St. Louis, MO (US)

(72) Inventors: Zebin Wang, St. Louis, MO (US); Tam H. Tran, St. Louis, MO (US); Yeun S. Gu, St. Louis, MO (US)

(21) Appl. No.: 14/417,433

(22) PCT Filed: Jul. 26, 2013
(86) PCT No.: PCT/US2013/052398

§ 371 Date: Jan. 26, 2015

Related U.S. Application Data
(60) Provisional application No. 61/676,032, filed on Jul. 26, 2012.

(51) Int. Cl. B01F 17/00; A61K 8/04; A61K 8/64; A61Q 11/00; C11D 3/32; A61Q 1/14; A61Q 19/10; A61Q 5/02; C11D 11/00; C11D 3/00; C09K 21/14; A61Q 9/02

(52) U.S. Cl. CPC B01F 17/005; C09K 21/14; A61K 8/046; A61K 8/645; A61Q 11/00; A61Q 9/02; A61Q 1/14; A61Q 19/10; A61Q 5/02; C11D 11/0017; C11D 3/0094; C11D 3/32

(57) ABSTRACT

A foaming agent comprising an amount of soy whey protein is disclosed herein, the soy whey protein having been isolated from processing streams. The foaming agent is especially suitable for producing a personal care product or an industrial product.

### CROSS-REFERENCE TO RELATED APPLICATIONS

This patent application claims the benefit of U.S. Provisional Application Ser. No. 61/676,032, filed on Jul. 26, 2012, which is incorporated by reference herein in its entirety.

### FIELD OF THE INVENTION

The present disclosure relates to a foaming agent for use in personal care products and industrial products. Specifically, the foaming agent comprises an amount of soy whey protein having a soluble solids index (SSI) of at least about 80% across a pH range of from 2 to 10 and a temperature of 25° C.

### BACKGROUND OF THE INVENTION

Foam products for personal care and industrial use have become one of the fastest growing processing operations for the development of new innovative products. Aeration of products into foams has several benefits over non-foam products. First, foam increases the accuracy and precision of product application. Also, foam has the ability to expand in order to extend coverage of the product into areas that would otherwise be missed by a non-foaming product. Finally, because foam has the ability to expand, less of the product needs to be applied in a single application, which enables consumers to extend the life of the product and effectively get more for their money.

Foaming agents are used in many industrial processes, including for products used around the home, for personal care, and in a variety of industries. A foaming agent is a material that facilitates the formation of foam in a mixture. Foamability is the capacity of a foaming agent to incorporate air into liquid. A foaming agent will exhibit good foamability if it rapidly adsorbs onto the air-water interface. Most foaming agents typically used in the art exhibit good foamability because they work to lower the surface tension of water, which is also the goal of foaming agents. Foaming agents typically used in the art are low molecular weight compounds (such as small molecular surfactants), which are normally less than about 10 kilodaltons (kDa). Smaller compounds typically have good foamability because they have a high surface activity and are able to lower the surface tension of water better and more rapidly than high molecular weight compounds.

Foam stability refers to the rate of foam to lose liquid and/or the breakup of gas bubbles. High molecular weight compounds, for example, mild hydrolyzed keratin, unfold in the liquid-air interface once they are adsorbed onto the interface and form inter-molecular bonds, which results in stable film to prevent gas bubble from breakup. However, because of the high molecular weight, it is difficult for the molecules to adsorb onto the liquid-air interface, therefore the foamability is poor. If the high molecular proteins are highly hydrolyzed, the resulted small molecular fragments will behave like other small molecular surface active compounds. That is, the molecules may easily adsorb to liquid-air interface thus exhibiting high surface activity and good foamablity, but unable to form stable film, which greatly reduce foam stability.

High molecular weight compounds, for example, guar gum, are commonly used as foam stabilizers because such hydrocolloids unfold and align themselves at the liquid-gas interface of the bubbles reinforcing the bubble walls. However, because high molecular weight compounds exhibit low surface activity, they do not work well as foaming agents. Likewise, small molecular weight compounds do not typically work well to stabilize foams.

Foaming agents (typically, surfactants) are routinely added to personal care and industrial products to provide foam forming capability. Typically, for specific industrial foam uses, surfactants are needed to reduce the surface tension of liquid. Examples of commonly used foaming agents typically used in the art include, but are not limited to, mono- and diglycerides of fatty acids, esters of monoglycerides of fatty acids, propylene glycol monoesters, lecithin, hydroxylated lecithin, dioctyl sodium sulphosuccinate, sodium stearoyl-2-lactylate (SSL), calcium stearoyl lactate (CSL), sorbitan monolaurate (Polysorbate 20 or Tween20), sorbitan monopalmitate (Polysorbate 40 or Tween40), sorbitan monostearate (Polysorbate 60 or Tween60), sorbitan monooleate (Polysorbate 80 or Tween80), sorbitan tristearate, stearyl citrate, polyglycerol polyricinoleate (PGPR), lactylates, sodium lauryl ether sulfate (SLES), sodium dodecyl sulfate (SDS), ammonium lauryl sulfate (ALS), cocamide diethanolamine, triethanolamine, and sodium lauroyl sarcosinate. Some commonly used foaming agents are detergents and can be irritating when used on skin. It would be desirable to use protein based foaming agents disclosed herein in conjunction with or as a replacement for commonly used foaming agents in order to provide benefits to personal care products and industrial products, for example, less irritating side effects when placed in contact with skin.

An ideal foaming agent would be one that has a high surface activity to provide good foamability but also provides long-term foam stability. Small molecular weight surfactants have high surface activity, thereby providing good foamability, but fail to provide long-term foam stability. High molecular weight biopolymers, such as proteins and carbohydrates, have low surface activity, which does not promote good foamability, but they can provide long-term foam stability.

Thus, there is a need in the art for a foaming agent that contains a protein based substance and that provides both good foamability and long-term foam stability and is biodegradable. Accordingly, the present invention is directed to a foaming agent comprised in whole or in part of soy whey protein for use in an industrial product, thereby eliminating or reducing the need to add one or more additional foaming agents to the product.

### SUMMARY OF THE INVENTION

The present disclosure relates to a foaming agent for use in personal care products and industrial products. Specifically, the foaming agent comprises an amount of soy whey protein having a soluble solids index (SSI) of at least about 80% across a pH range of from 2 to 10 and a temperature of 25° C. The inclusion of soy whey protein as a foaming agent acts to provide long-term foam stability for the personal care products and industrial products.

The present disclosure further relates to personal care products and industrial products that contain a foaming agent comprising an amount of soy whey protein having a soluble solids index (SSI) of at least about 80% across a pH range of from 2 to 10 and a temperature of 25° C. The foaming agent disclosed herein is suitable for use in the preparation of various types of personal care products and industrial products that require some degree of aeration, for example, personal care products (such as hand soap, face soap, body soap, shaving foam, toothpaste, shampoo, hair color product, and hair styling foam), household products, flame retardant agent, foam insulation, foam mattress, foam rubber, foam packaging, and the like.

The present disclosure further relates to a method of making an industrial product, the method comprising combining a foaming agent with water and/or other ingredients to form an aerated mixture and processing the aerated mixture into the desired product, wherein the foaming agent comprises an amount of soy whey protein having been recovered from a processing stream and having a soluble solids index (SSI) of at least about 80% across a pH range of from 2 to 10 and a temperature of 25° C.

### BRIEF DESCRIPTION OF THE DRAWINGS

FIG. 1 is a chart setting forth the proteins found in soy whey streams and their characteristics.

FIG. 2 graphically depicts the solubility of the soy whey proteins over a pH range of 3-7 as compared to that of soy protein isolates.

FIG. 3 graphically depicts the rheological properties of the soy whey proteins compared to soy protein isolate, Supro® 760.

FIG. 4A is a schematic flow sheet depicting Steps 0 through 4 in a process for recovery of a purified soy whey protein from processing stream.

FIG. 4B is a schematic flow sheet depicting Steps 5, 6, 14, 15, 16, and 17 in a process for recovery of a purified soy whey protein from processing stream.

FIG. 4C is a schematic flow sheet depicting Steps 7 through 13 in a process for recovery of a purified soy whey protein from processing stream.

FIG. 5 graphically illustrates the breakthrough curve when loading soy whey at 10, 15, 20 and 30 mL/min (5.7, 8.5, 11.3, 17.0 cm/min linear flow rate, respectively) through a SP Gibco cation exchange resin bed plotted against empty column volumes loaded.

FIG. 6 graphically illustrates protein adsorption on SP Gibco cation exchange resin when passing soy whey at 10, 15, 20 and 30 mL/min (5.7, 8.5, 11.3, 17.0 cm/min linear flow rate, respectively) plotted against empty column volumes loaded.

FIG. 7 graphically illustrates the breakthrough curve when loading soy whey at 15 mL/min and soy whey concentrated by a factor of 3 and 5 through SP Gibco cation exchange resin bed plotted against empty column volumes loaded.

FIG. 8 graphically illustrates protein adsorption on SP Gibco cation exchange resin when passing soy whey and soy whey concentrated by a factor of 3 and 5 at 15 mL/min through SP Gibco cation exchange resin bed plotted against empty column volumes loaded.

FIG. 9 graphically depicts equilibrium protein adsorption on SP Gibco cation exchange resin when passing soy whey and soy whey concentrated by a factor of 3 and 5 at 15 mL/min through SP Gibco cation exchange resin bed plotted against equilibrium protein concentration in the flow through.

FIG. 10 graphically illustrates the elution profiles of soy whey proteins desorbed with varying linear velocities over time.

FIG. 11 graphically illustrates the elution profiles of soy whey proteins desorbed with varying linear velocities with column volumes.

FIG. 12 depicts a sodium dodecyl sulfate polyacrylamide gel electrophoresis (SDS-PAGE) analysis of Mimo6ME fractions.

FIG. 13 depicts a SDS-PAGE analysis of Mimo4SE fractions.

FIG. 14 depicts a SDS-PAGE analysis of Mimo6HE fractions.

FIG. 15 depicts a SDS-PAGE analysis of Mimo6ZE fractions.

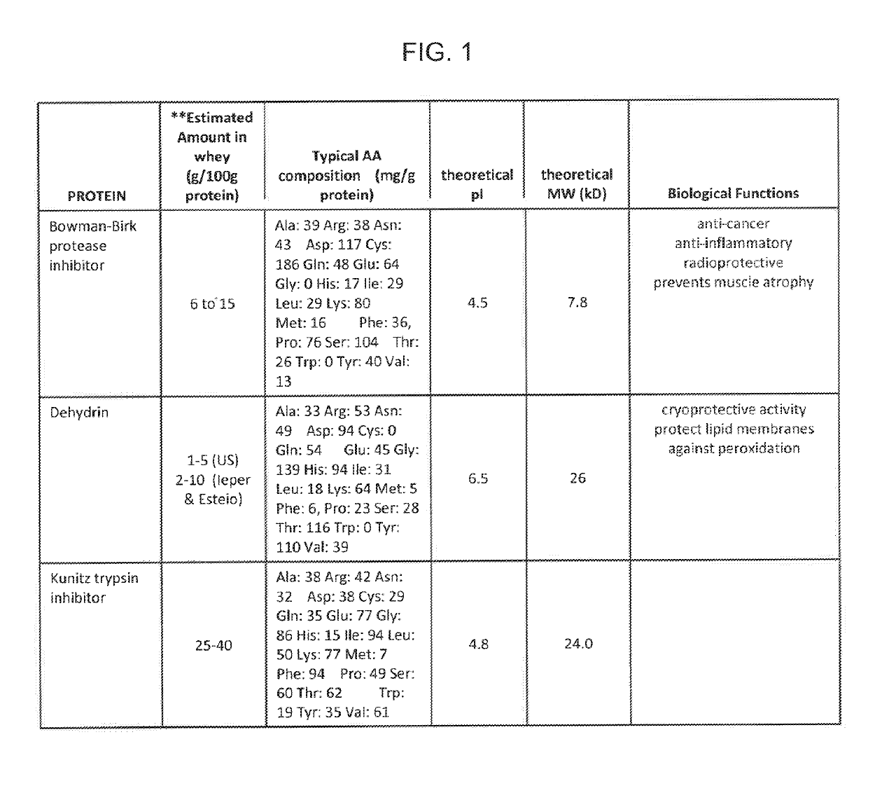

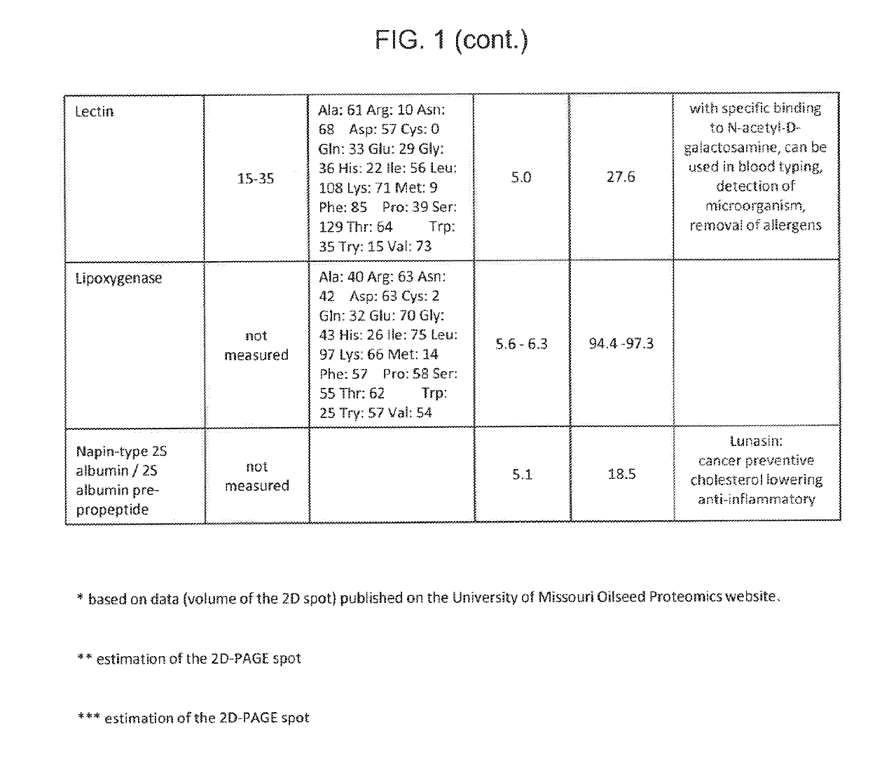

### DETAILED DESCRIPTION OF THE PREFERRED ASPECTS

The present invention provides a foaming agent comprising an amount of soy whey protein having a soluble solids index (SSI) of at least about 80% across a pH range of from 2 to 10 and a temperature of 25° C. The foaming agent, when added to personal care products and industrial products, is biodegradable and imparts foaming properties (i.e., foamability and foam stability) when comparing the resultant products to similar personal care products and industrial products in the market which contain commonly used foaming agents.

### I. Foaming Agent

The foaming agent of the present invention for use in personal care products and industrial products contains an amount of soy whey protein having a soluble solids index (SSI) of at least about 80% across a pH range of from 2 to 10 and a temperature of 25° C.

The soy whey proteins of the present invention have been discovered to impart excellent foaming properties (i.e., foamability and foam stability) when used in industrial compositions over foaming agents currently used in the art. It has been surprisingly discovered that while soy whey proteins are high molecular weight compounds (e.g., about 8 kDa to about 50 kDa), they possess the desired characteristics of both small molecular weight foaming agents and large molecular weight foaming agents. The soy whey proteins have a high molecular weight thus they are able to provide long-term foam stability but surprisingly behave as small molecular weight compounds (i.e., good foamability) in that they promote a reduction in surface tension.

In one embodiment, the foaming agent of the present invention contains 100% soy whey protein. In another embodiment, the foaming agent contains a combination of soy whey protein and at least one additional foaming agent. For instance, the foaming agent may comprise soy whey protein and at least one additional foaming agent selected from the group consisting of mono- & diglycerides of fatty acids, esters of monoglycerides of fatty acids, propylene glycol monoesters, lecithin, hydroxylated lecithin, dioctyl sodium sulphosuccinate, SSL, CSL, Polysorbate 20, Polysorbate 40, Polysorbate 60, Polysorbate 80, sorbitan tristearate, stearyl citrate, PGPR, lactylates, SLES, SDS, ALS, cocamide diethanolamine, triethanolamine, INCl, and combinations thereof. For example, the foaming agent may contain between about 5% to about 99.9% (w/w) of soy whey protein. Specifically, the foaming agent of the present invention may contain about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 98.5%, 99%, 99.5%, 99.6%, 99.7%, 99.8%, or 99.9% (w/w) of soy whey protein.

### II. Soy Whey Proteins

The soy whey proteins of the present disclosure represent a significant advance in the art over other soy proteins and isolates. As noted herein, the soy whey proteins of the present disclosure possess unique characteristics as compared to other soy proteins found in the art.

Soy protein isolates are typically precipitated from an aqueous extract of defatted soy flakes or soy flour at the isoelectric point of soy storage proteins (e.g. a pH of about 4.5±.5). Thus, soy protein isolates generally include proteins that are not soluble in acidic liquid media. Similarly, the proteins of soy protein concentrates, the second-most refined soy protein material, are likewise generally not soluble in acidic liquid media. However, soy whey proteins of the present disclosure differ in that they are generally acid-soluble, meaning they are soluble in acidic liquid media.

The present disclosure provides soy whey protein compositions derived from an aqueous soy whey that exhibit advantageous characteristics over soy proteins found in the prior art.

A. High Solubility

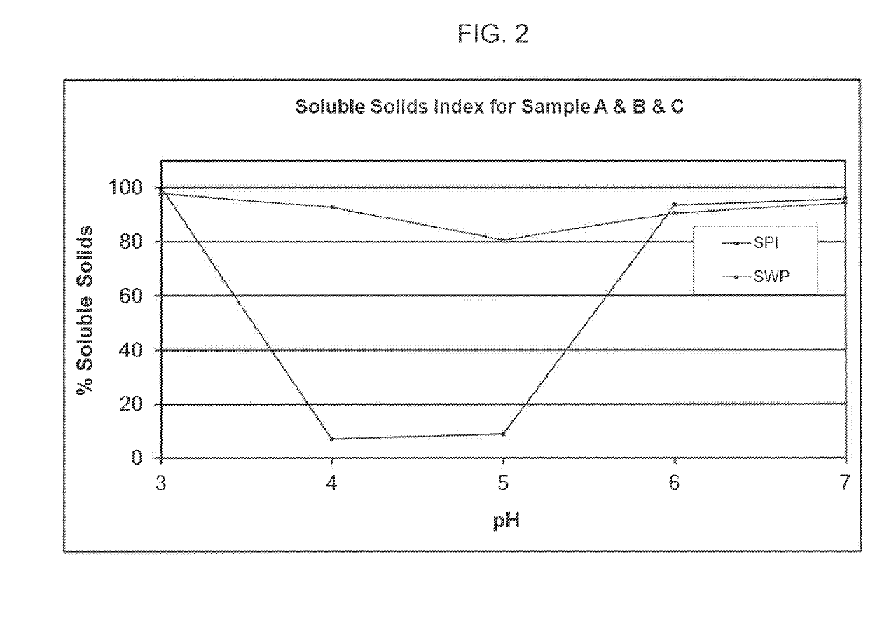

The soy whey proteins isolated according to the methods of the present invention possess high solubility (i.e. SSI % greater than 80) across a relatively wide pH range of the aqueous (typically acidic) medium (e.g. an aqueous medium having a pH of from about 2 to about 10, from about 2 to about 7, or from about 2 to about 6) at ambient conditions (e.g. a temperature of about 25° C.). As shown in Table 1 and graphically illustrated in FIG. 2, the solubility of the soy whey proteins isolated in accordance with the methods of the present disclosure, at all pH values tested, was at least 80%, and in all but one instance (i.e. pH 4) was at least about 90%. These findings were compared with soy protein isolate, which was shown to display poor solubility characteristics at the same acid pH values. This unique characteristic enables the soy whey proteins of the present invention to be used in applications having acidic pH levels, which represents a significant advantage over soy isolate.

B. Low Viscosity

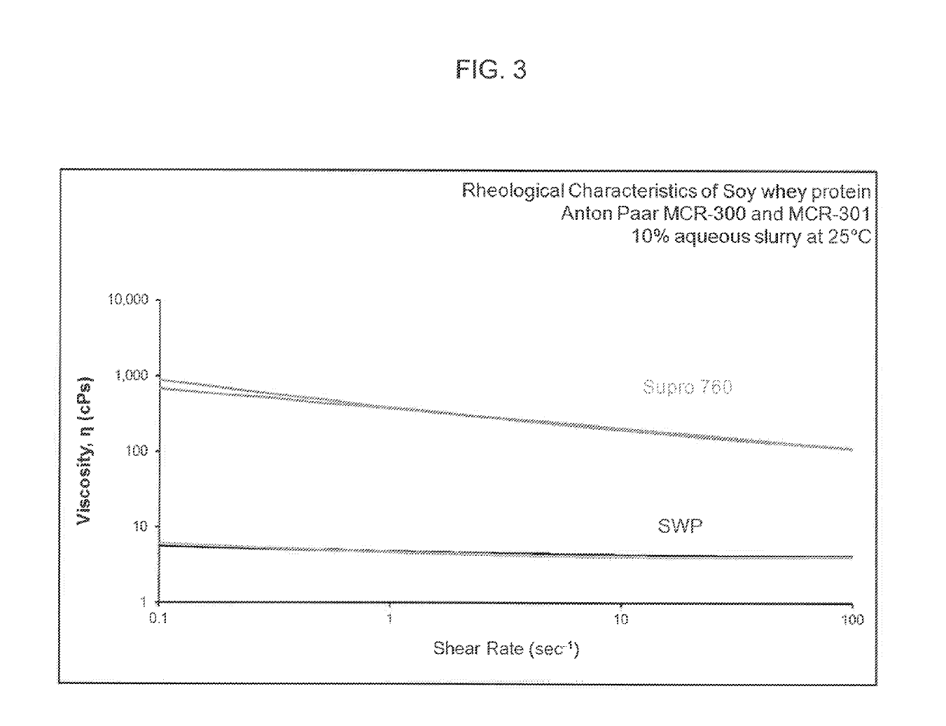

In addition to solubility, the soy whey proteins of the present disclosure also possess much lower viscosity than other soy proteins. As shown in Table 1 and as graphically illustrated in FIG. 3, the soy whey proteins of the present invention displayed viscoelastic properties (i.e. rheological properties) more similar to that of water than shown by soy protein isolate. The viscosity of water is about 1 centipoise (cP) at 20° C. The soy whey proteins of the present disclosure were found to exhibit viscosity within the range of from about 2.0 to 10.0 cP, and preferably from about 3.6 to 7.5 cP. This low viscosity, in addition to its high solubility at acidic pH levels, makes the soy whey protein of the present disclosure available and better suited for use in certain applications that regularly involve the use of other soy proteins (e.g., in personal care products and industrial products), because it has much better flow characteristics than that of soy isolate.

TABLE 1
Solubility and Viscoelastic Properties of Soy Whey Protein
(SWP) Compared to a Commercial Isolated Soy Protein

| | SWP | Supro ® 500E |
|---|---|---|
| SSI %, pH 3.0 | 99 | 100 |
| SSI %, pH 4.0 | 82.3 | 7 |
| SSI %, pH 5.0 | 89.4 | 9 |
| SSI %, pH 6.0 | 99.3 | 94 |
| SSI %, pH 7.0 | 99.4 | 96 |
| Viscosity*, cPs | 4.3 | 5700-7100 |

*Viscosity was measured on isolate in water slurries at as is pH. Concentration at 10% w/w (weight isolate/total sample weight). Temperature at 25° C. Shear rate was 1 1/s (second).

As Table 2 illustrates, the other physical characteristics, with the exception of the viscoelastic properties and solubility, of the soy whey protein recovered in accordance with the methods of the present disclosure were found to be very similar to that of soy isolate.

TABLE 2
Physical Characteristic Ranges of Soy
Whey Proteins from Two Plant Sites

| | ranges, | ranges, | combined |
|---|---|---|---|
| | leper | St. Louis | SWP range |
| moisture | 2.94-9.34 | 3.91-8.29 | 2.9-9.4 |
| protein_db | 71.0-89.3 | 62.48-85.17 | 62.4-89.3 |
| ash_ db | 1.19-6.23 | 1.19-13.57 | 1.19-13.57 |
| fat_db | 0.201-1.11 | 0.14-1.57 | 0.14-1.57 |
| carbohydrate | 7.2-23.7 | 5.4-30.5 | 5.4-30.5 |
| by diff _ db | (10 & 20 kDa | | |
| | membrane) | | |

| | leper | St. Louis | combined |
|---|---|---|---|
| | SWP | SWP | range |
| SSI %, pH 3.0 | 79-99 | 71.6-100 | 71-100 |
| SSI %, pH 4.0 | 68.7-97.3 | 67.4-94.7 | 67-98 |
| SSI %, pH 5.0 | 70.4-88.9 | 69.4-91.5 | 69-92 |
| SSI %, pH 6.0 | 79.1-93.49 | 75.1-100 | 75-100 |
| SSI %, pH 7.0 | 77.6-97.2 | 79.6-100 | 77-100 |
| viscosity, cPs | 3.6-7.5 | 3.3 | 3.3-7.5 |
| | | (1 sample only) | |

### III. Aqueous Whey Streams

Aqueous whey streams and molasses streams, which are types of soy processing streams, are generated from the process of refining a whole legume or oilseed. The whole legume or oilseed may be derived from a variety of suitable plants. By way of non-limiting example, suitable plants include leguminous plants, including for example, soybeans, corn, peas, canola, sunflowers, sorghum, rice, amaranth, potato, tapioca, arrowroot, canna, lupin, rape, wheat, oats, rye, barley, and mixtures thereof. In one embodiment, the leguminous plant is soybean and the aqueous whey stream generated from the process of refining the soybean is an aqueous soy whey stream.

Aqueous soy whey streams generated in the manufacture of soy protein isolates are generally relatively dilute and are typically discarded as waste. More particularly, the aqueous soy whey stream typically has a total solids content of less than about 10 wt. %, typically less than about 7.5 wt. % and, still more typically, less than about 5 wt. %. For example, in various aspects, the solids content of the aqueous soy whey stream is from about 0.5 to about 10 wt. %, from about 1 wt. % to about 4 wt. %, or from about 1 to about 3 wt. % (e.g. about 2 wt. %). Thus, during commercial soy protein isolate production, a significant volume of waste water that must be treated or disposed is generated.

Soy whey streams typically contain a significant portion of the initial soy protein content of the starting material soybeans. As used herein the term “soy protein” generally refers to any and all of the proteins native to soybeans. Naturally occurring soy proteins are generally globular proteins having a hydrophobic core surrounded by a hydrophilic shell. Numerous soy proteins have been identified including, for example, storage proteins such as glycinin and β-conglycinin. Soy proteins likewise include protease inhibitors, such as the above-noted BBI proteins. Soy proteins also include hemagglutinins such as lectin, lipoxygenases, β-amylase, and lunasin. It is to be noted that the soy plant may be transformed to produce other proteins not normally expressed by soy plants. It is to be understood that reference herein to “soy proteins” likewise contemplates proteins thus produced.

On a dry weight basis, soy proteins constitute at least about 10 wt. %, at least about 15 wt. %, or at least about 20 wt. % of the soy whey stream (dry weight basis). Typically, soy proteins constitute from about 10 to about 40 wt. %, or from about 25 to about 30 wt. % of the soy whey stream (dry weight basis). Soy protein isolates typically contain a significant portion of the storage proteins of the soybean. However, the soy whey stream remaining after isolate precipitation likewise contains one or more soy storage proteins.

In addition to the various soy proteins, the aqueous soy whey stream likewise comprises one or more carbohydrates (i.e. sugars). Generally, sugars constitute at least about 25%, at least about 35%, or at least about 45% by weight of the soy whey stream (dry weight basis). Typically, sugars constitute from about 25% to about 75%, more typically from about 35% to about 65% and, still more typically, from about 40% to about 60% by weight of the soy whey stream (dry weight basis).

The sugars of the soy whey stream generally include one or more monosaccharides, and/or one or more oligosaccharides or polysaccharides. For example, in various aspects, the soy whey stream comprises monosaccharides selected from the group consisting of glucose, fructose, and combinations thereof. Typically, monosaccharides constitute from about 0.5% to about 10 wt. % and, more typically from about 1% to about 5 wt. % of the soy whey stream (dry weight basis). Further in accordance with these and various other aspects, the soy whey stream comprises oligosaccharides selected from the group consisting of sucrose, raffinose, stachyose, and combinations thereof. Typically, oligosaccharides constitute from about 30% to about 60% and, more typically, from about 40% to about 50% by weight of the soy whey stream (dry weight basis).

The aqueous soy whey stream also typically comprises an ash fraction that includes a variety of components including, for example, various minerals, isoflavones, phytic acid, citric acid, saponins, and vitamins. Minerals typically present in the soy whey stream include sodium, potassium, calcium, phosphorus, magnesium, chloride, iron, manganese, zinc, copper, and combinations thereof. Vitamins present in the soy whey stream include, for example, thiamine and riboflavin. Regardless of its precise composition, the ash fraction typically constitutes from about 5% to about 30% and, more typically, from about 10% to about 25% by weight of the soy whey stream (dry weight basis).

The aqueous soy whey stream also typically comprises a fat fraction that generally constitutes from about 0.1% to about 5% by weight of the soy whey stream (dry weight basis). In certain aspects of the invention, the fat content is measured by acid hydrolysis and is about 3% by weight of the soy whey stream (dry weight basis).

In addition to the above components, the aqueous soy whey stream also typically comprises one or more microorganisms including, for example, various bacteria, molds, and yeasts. The proportions of these components typically vary from about 100 to about 1×10⁹ colony forming units (CFU) per milliliter. As detailed elsewhere herein, in various aspects, the aqueous soy whey stream is treated to remove these component(s) prior to protein recovery and/or isolation.

As noted, conventional production of soy protein isolates typically includes disposal of the aqueous soy whey stream remaining following isolation of the soy protein isolate. In accordance with the present disclosure, recovery of one or more proteins and various other components (e.g. sugars and minerals) results in a relatively pure aqueous whey stream. Conventional soy whey streams from which the protein and one or more components have not been removed generally require treatment prior to disposal and/or reuse. In accordance with various aspects of the present disclosure the aqueous whey stream may be disposed of or utilized as process water with minimal, if any, treatment. For example, the aqueous whey stream may be used in one or more filtration (e.g. diafiltration) operations of the present disclosure.

In addition to recovery of BBI proteins from aqueous soy whey streams generated in the manufacture of soy protein isolates, it is to be understood that the processes described herein are likewise suitable for recovery of one or more components of soy molasses streams generated in the manufacture of a soy protein concentrate, as soy molasses streams are an additional type of soy processing stream.

### IV. General Description of Process for Soy Whey Protein Recovery

Generally, the purification of the soy processing stream comprises one or more operations (e.g. membrane separation operations) selected and designed to provide recovery of the desired proteins or other products, or separation of various components of the soy whey stream, or both. Recovery of soy whey proteins (e.g. Bowman-Birk inhibitor (BBI) and Kunitz trypsin inhibitor (KTI) proteins) and one or more other components of the soy whey stream (e.g. various sugars, including oligosaccharides) may utilize a plurality of separation techniques, (e.g. membrane, chromatographic, centrifugation, or filtration). The specific separation technique will depend upon the desired component to be recovered by separating it from other components of the processing stream.

For example, a purified fraction is typically prepared by removal of one or more impurities (e.g. microorganisms or minerals), followed by removal of additional impurities including one or more soy storage proteins (i.e. glycinin and β-conglycinin), followed by removal of one or more soy whey proteins (including, for example, KTI and other non-BBI proteins or peptides), and/or followed by removal of one or more additional impurities including sugars from the soy whey. Recovery of various target components in high purity form is improved by removal of other major components of the whey stream (e.g. storage proteins, minerals, and sugars) that detract from purity by diluents, while likewise improving purity by purifying the protein fraction through removal of components that are antagonists to the proteins and/or have deleterious effects (e.g. endotoxins). Removal of the various components of the soy whey typically comprises concentration of the soy whey prior to and/or during removal of the components of the soy whey. The methods of the present invention also will reduce pollution generated from processing large quantities of aqueous waste.

Removal of storage proteins, sugars, minerals, and impurities yields fractions that are enriched in the individual, targeted proteins and free of impurities that may be antagonists or toxins, or may otherwise have a deleterious effect. For example, typically a soy storage protein-enriched fraction may be recovered, along with a fraction enriched in one or more soy whey proteins. A fraction enriched in one more sugars (e.g. oligosaccharides and/or polysaccharides) is also typically prepared. Thus, the present methods provide a fraction that is suitable as a substrate for recovery of individual, targeted proteins, and also provide other fractions that can be used as substrates for economical recovery of other useful products from aqueous soy whey. For example, removal of sugars and/or minerals from the soy whey stream produces a useful fraction from which the sugars can be further separated, thus yielding additional useful fractions: a concentrated sugar and a mineral fraction (that may include citric acid), and a relatively pure aqueous fraction that may be disposed of with minimal, if any, treatment or recycled as process water. Process water thus produced may be especially useful in practicing the present methods. Thus, a further advantage of the present methods may be reduced process water requirements as compared to conventional isolate preparation processes.

Methods of the present disclosure provide advantages over conventional methods for manufacture of soy protein isolates and concentrates in at least two ways. As noted, conventional methods for manufacturing soy protein materials typically dispose of the soy whey stream (e.g. aqueous soy whey or soy molasses). Thus, the products recovered by the methods of the present disclosure represent an additional product, and a revenue source not currently realized in connection with conventional soy protein isolate and soy protein concentrate manufacture. Furthermore, treatment of the soy whey stream or soy molasses to recover saleable products preferably reduces the costs associated with treatment and disposal of the soy whey stream or soy molasses. For example, as detailed elsewhere herein, various methods of the present invention provide a relatively pure soy processing stream that may be readily utilized in various other processes or disposed of with minimal, if any, treatment, thereby reducing the environmental impact of the process. Certain costs exist in association with the methods of the present disclosure, but the benefits of the additional product(s) isolated and minimization of waste disposal are believed to compensate for any added costs.

The following is a general description of the various steps that make up the overall process. A key to the process is to start with the whey protein pretreatment step, which uniquely changes the soy whey and protein properties. From there, the other steps may be performed using the raw material sources as listed in each step, as will be shown in the discussion of the various embodiments to follow.

It is understood by those skilled in the art of separation technology that there can be residual components in each permeate or retentate stream since separation is never 100%. Further, one skilled in the art realizes that separation technology can vary depending on the starting raw material.

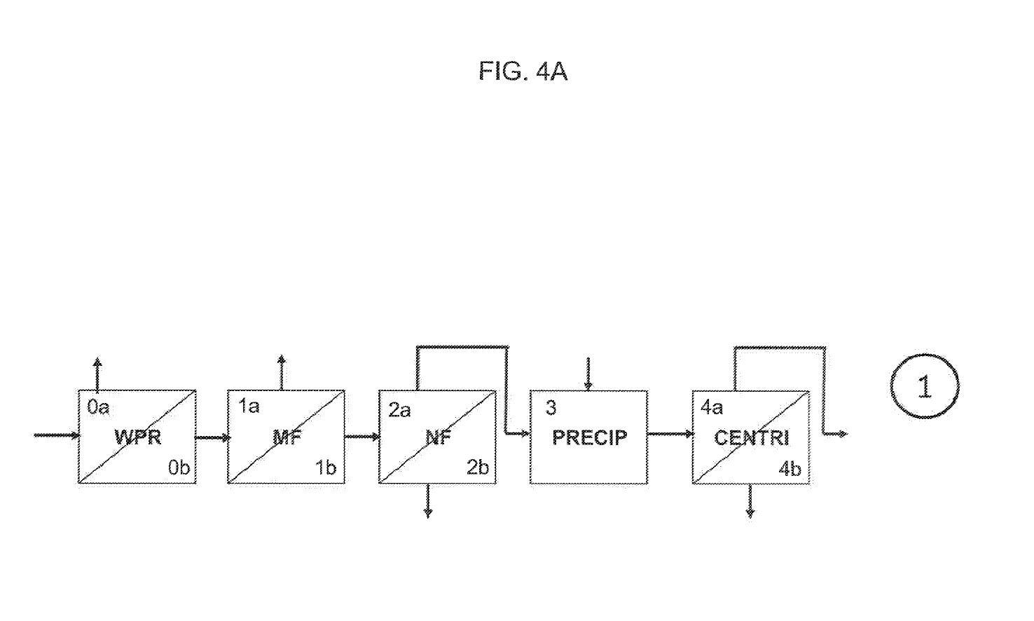

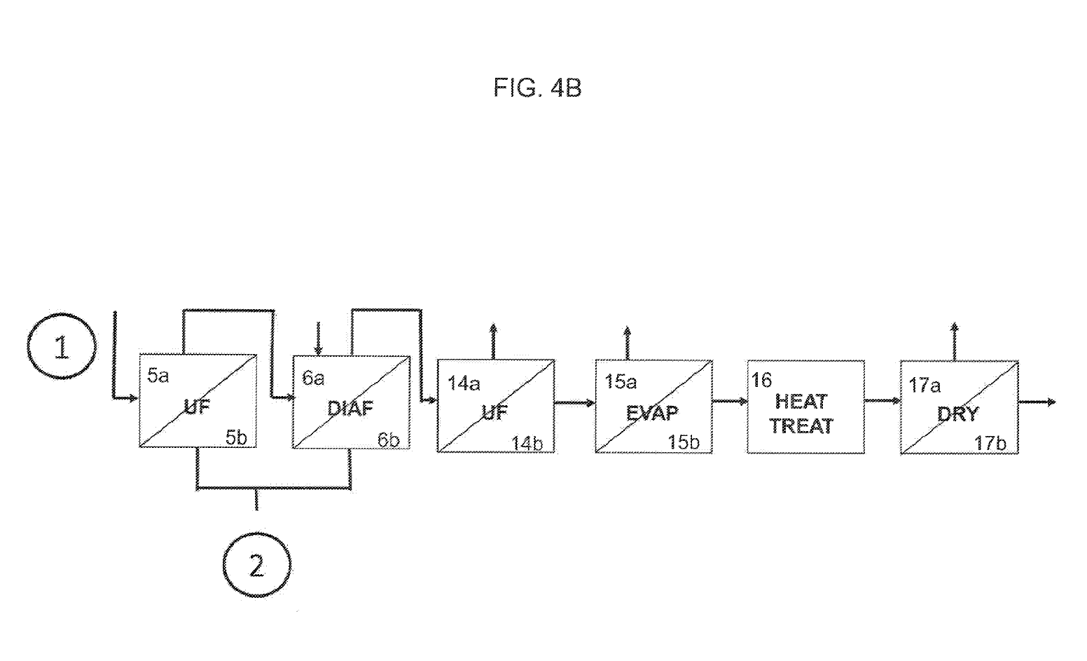

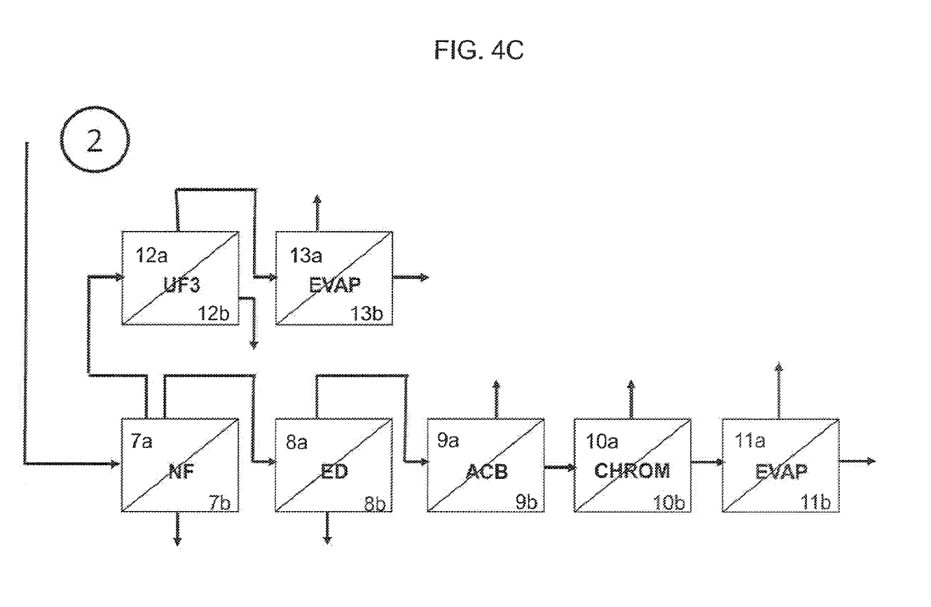

Step 0 (as shown in FIG. 4A)—Whey protein pretreatment can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 can be between about 3.0 and about 6.0, preferably 4.5. The temperature can be between about 70° C. and about 95° C., preferably about 85° C. Temperature hold times can vary between about 0 minutes to about 20 minutes, preferably about 10 minutes. Products from the whey protein pretreatment include but are not limited to soluble components in the aqueous phase of the whey stream (pre-treated soy whey) (molecular weight of equal to or less than about 50 kDa in stream 0a (retentate) and insoluble large molecular weight proteins (between about 300 kD and between about 50 kD) in stream 0b (permeate), such as pre-treated soy whey, storage proteins, and combinations thereof.

Step 1 (as shown in FIG. 4A)—Microbiology reduction can start with the product of the whey protein pretreatment step, including but not limited to pre-treated soy whey. This step involves microfiltration of the pre-treated soy whey. Process variables and alternatives in this step include but are not limited to, centrifugation, dead-end filtration, heat sterilization, ultraviolet sterilization, microfiltration, cross flow membrane filtration, and combinations thereof. Crossflow membrane filtration includes but is not limited to: spiral-wound, plate and frame, hollow fiber, ceramic, dynamic or rotating disk, nanofiber, and combinations thereof. The pH of step 1 can be between about 2.0 and about 12.0, preferably about 5.3. The temperature can be between about 5° C. and about 90° C., preferably about 50° C. Products from step 1 include but are not limited to storage proteins, microorganisms, silicon, and combinations thereof in stream 1a (retentate) and purified pre-treated soy whey in stream 4b (permeate).

Step 2 (as shown in FIG. 4A)—A water and mineral removal can start with the purified pre-treated soy whey from stream 1b or 4a, or pre-treated soy whey from stream 0b. It includes a nanofiltration step for water removal and partial mineral removal. Process variables and alternatives in this step include but are not limited to, crossflow membrane filtration, reverse osmosis, evaporation, nanofiltration, and combinations thereof. Crossflow membrane filtration includes but is not limited to: spiral-wound, plate and frame, hollow fiber, ceramic, dynamic or rotating disk, nanofiber, and combinations thereof. The pH of step 2 can be between about 2.0 and about 12.0, preferably about 5.3. The temperature can be between about 5° C. and about 90° C., preferably about 50° C. Products from this water removal step include but are not limited to purified pre-treated soy whey in stream 2a (retentate) and water, some minerals, monovalent cations and combinations thereof in stream 2b (permeate).

Step 3 (as shown in FIG. 4A)—the mineral precipitation step can start with purified pre-treated soy whey from stream 2a or pretreated soy whey from streams 0a or 1b. It includes a precipitation step by pH and/or temperature change. Process variables and alternatives in this step include but are not limited to, an agitated or recirculating reaction tank. Processing aids that can be used in the mineral precipitation step include but are not limited to, acids, bases, calcium hydroxide, sodium hydroxide, hydrochloric acid, sodium chloride, phytase, and combinations thereof. The pH of step 3 can be between about 2.0 and about 12.0, preferably about 8.0. The temperature can be between about 5° C. and about 90° C., preferably about 50° C. The pH hold times can vary between about 0 minutes to about 60 minutes, preferably about 10 minutes. The product of stream 3 is a suspension of purified pre-treated soy whey and precipitated minerals.

Step 4 (as shown in FIG. 4A)—the mineral removal step can start with the suspension of purified pre-treated whey and precipitated minerals from stream 3. It includes a centrifugation step. Process variables and alternatives in this step include but are not limited to, centrifugation, filtration, dead-end filtration, crossflow membrane filtration and combinations thereof. Crossflow membrane filtration includes but is not limited to: spiral-wound, plate and frame, hollow fiber, ceramic, dynamic or rotating disk, nanofiber, and combinations thereof. Products from the mineral removal step include but are not limited to a de-mineralized pre-treated whey in stream 4a (retentate) and insoluble minerals with some protein mineral complexes in stream 4b (permeate).

Step 5 (as shown in FIG. 4B)—the protein separation and concentration step can start with purified pre-treated whey from stream 4a or the whey from streams 0a, 1b, or 2a. It includes an ultrafiltration step. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, preferably about 8.0. The temperature can be between about 5° C. and about 90° C., preferably about 75° C. Products from stream 5a (retentate) include but are not limited to, soy whey protein, BBI, KTI, storage proteins, other proteins and combinations thereof. Other proteins include but are not limited to lunasin, lectins, dehydrins, lipoxygenase, and combinations thereof. Products from stream 5b (permeate) include but are not limited to, peptides, soy oligosaccharides, minerals and combinations thereof. Soy oligosaccharides include but are not limited to sucrose, raffinose, stachyose, verbascose, monosaccharides, and combinations thereof. Minerals include but are not limited to calcium citrate.

Step 6 (as shown in FIG. 4B)—the protein washing and purification step can start with soy whey protein, BBI, KTI, storage proteins, other proteins or purified pre-treated whey from stream 4a or 5a, or whey from streams 0a, 1b, or 2a. It includes a diafiltration step. Process variables and alternatives in this step include but are not limited to, reslurrying, crossflow membrane filtration, ultrafiltration, water diafiltration, buffer diafiltration, and combinations thereof. Crossflow membrane filtration includes but is not limited to: spiral-wound, plate and frame, hollow fiber, ceramic, dynamic or rotating disk, nanofiber, and combinations thereof. Processing aids that can be used in the protein washing and purification step include but are not limited to, water, steam, and combinations thereof. The pH of step 6 can be between about 2.0 and about 12.0, preferably about 7.0. The temperature can be between about 5° C. and about 90° C., preferably about 75° C. Products from stream 6a (retentate) include but are not limited to, soy whey protein, BBI, KTI, storage proteins, other proteins, and combinations thereof. Other proteins include but are not limited to lunasin, lectins, dehydrins, lipoxygenase, and combinations thereof. Products from stream 6b (permeate) include but are not limited to, peptides, soy oligosaccharides, water, minerals, and combinations thereof. Soy oligosaccharides include but are not limited to sucrose, raffinose, stachyose, verbascose, monosaccharides, and combinations thereof. Minerals include but are not limited to calcium citrate.

Step 7 (as shown in FIG. 4C)—a water removal step can start with peptides, soy oligosaccharides, water, minerals, and combinations thereof from stream 5b and/or stream 6b. Soy oligosaccharides include but are not limited to sucrose, raffinose, stachyose, verbascose, monosaccharides, and combinations thereof. It includes a nanofiltration step. Process variables and alternatives in this step include but are not limited to, reverse osmosis, evaporation, nanofiltration, water diafiltration, buffer diafiltration, and combinations thereof. The pH of step 7 can be between about 2.0 and about 12.0, preferably about 7.0. The temperature can be between about 5° C. and about 90° C., preferably about 50° C. Products from stream 7a (retentate) include but are not limited to, peptides, soy oligosaccharides, water, minerals, and combinations thereof. Soy oligosaccharides include but are not limited to sucrose, raffinose, stachyose, verbascose, monosaccharides, and combinations thereof. Products from stream 7b (permeate) include but are not limited to, water, minerals, and combinations thereof.

Step 8 (as shown in FIG. 4C)—a mineral removal step can start with peptides, soy oligosaccharides, water, minerals, and combinations thereof from streams 5b, 6b, 7a, and/or 12a. Soy oligosaccharides include but are not limited to sucrose, raffinose, stachyose, verbascose, monosaccharides, and combinations thereof. It includes an electrodialysis membrane step. Process variables and alternatives in this step include but are not limited to, ion exchange columns, chromatography, and combinations thereof. Processing aids that can be used in this mineral removal step include but are not limited to, water, enzymes, and combinations thereof. Enzymes include but are not limited to protease, phytase, and combinations thereof. The pH of step 8 can be between about 2.0 and about 12.0, preferably about 7.0. The temperature can be between about 5° C. and about 90° C., preferably about 40° C. Products from stream 8a (retentate) include but are not limited to, de-mineralized soy oligosaccharides with conductivity between about 10 milli Siemens (mS) and about 0.5 mS, preferably about 2 mS, and combinations thereof. Soy oligosaccharides include but are not limited to sucrose, raffinose, stachyose, verbascose, monosaccharides, and combinations thereof. Products from stream 8b include but are not limited to, minerals, water, and combinations thereof.

Step 9 (as shown in FIG. 4C)—a color removal step can start with de-mineralized soy oligosaccharides from streams 8a, 5b, 6b, and/or 7a). It utilizes an active carbon bed. Process variables and alternatives in this step include but are not limited to, ion exchange. Processing aids that can be used in this color removal step include but are not limited to, active carbon, ion exchange resins, and combinations thereof. The temperature can be between about 5° C. and about 90° C., preferably about 40° C. Products from stream 9a (retentate) include but are not limited to, color compounds. Stream 9b is decolored. Products from stream 9b (permeate) include but are not limited to, soy oligosaccharides, and combinations thereof. Soy oligosaccharides include but are not limited to sucrose, raffinose, stachyose, verbascose, monosaccharides, and combinations thereof.

Step 10 (as shown in FIG. 4C)—a soy oligosaccharide fractionation step can start with soy oligosaccharides, and combinations thereof from streams 9b, 5b, 6b, 7a, and/or 8a. Soy oligosaccharides include but are not limited to sucrose, raffinose, stachyose, verbascose, monosaccharides, and combinations thereof. It includes a chromatography step. Process variables and alternatives in this step include but are not limited to, chromatography, nanofiltration, and combinations thereof. Processing aids that can be used in this soy oligosaccharide fractionation step include but are not limited to acid and base to adjust the pH as one know in the art and related to the resin used. Products from stream 10a (retentate) include but are not limited to, soy oligosaccharides such as sucrose, monosaccharides, and combinations thereof. Products from stream 10b (permeate) include but are not limited to soy oligosaccharides such as, raffinose, stachyose, verbascose, and combinations thereof.

Step 11 (as shown in FIG. 4C)—a water removal step can start with soy oligosaccharides such as, raffinose, stachyose, verbascose, and combinations thereof from streams 9b, 5b, 6b, 7a, 8a, and/or 10a. It includes an evaporation step. Process variables and alternatives in this step include but are not limited to, evaporation, reverse osmosis, nanofiltration, and combinations thereof. Processing aids that can be used in this water removal step include but are not limited to, defoamer, steam, vacuum, and combinations thereof. The temperature can be between about 5° C. and about 90° C., preferably about 60° C. Products from stream 11a (retentate) include but are not limited to, water. Products from stream 11b (permeate) include but are not limited to, soy oligosaccharides, such as, raffinose, stachyose, verbascose, and combinations thereof.

Step 12 (as shown in FIG. 4C)—an additional protein separation from soy oligosaccharides step can start with peptides, soy oligosaccharides, water, minerals, and combinations thereof from stream 7b. Soy oligosaccharides include but are not limited to sucrose, raffinose, stachyose, verbascose, monosaccharides, and combinations thereof. It includes an ultrafiltration step. Process variables and alternatives in this step include but are not limited to, crossflow membrane filtration, ultrafiltration with pore sizes between about 50 kDa and about 1 kDa, and combinations thereof. Crossflow membrane filtration includes but is not limited to: spiral-wound, plate and frame, hollow fiber, ceramic, dynamic or rotating disk, nanofiber, and combinations thereof. Processing aids that can be used in this protein separation from sugars step include but are not limited to, acids, bases, protease, phytase, and combinations thereof. The pH of step 12 can be between about 2.0 and about 12.0, preferably about 7.0. The temperature can be between about 5° C. and about 90° C., preferably about 75° C. Products from stream 12a (retentate) include but are not limited to, soy oligosaccharides, water, minerals, and combinations thereof. Soy oligosaccharides include but are not limited to sucrose, raffinose, stachyose, verbascose, monosaccharides, and combinations thereof. Minerals include but are not limited to calcium citrate. This stream 12a stream can be fed to stream 8. Products from stream 12b (permeate) include but are not limited to, peptides, and other proteins. Other proteins include but are not limited to lunasin, lectins, dehydrins, lipoxygenase, and combinations thereof.

Step 13 (as shown in FIG. 4C)—a water removal step can start with, peptides, and other proteins. Other proteins include but are not limited to lunasin, lectins, dehydrins, lipoxygenase, and combinations thereof. It includes an evaporation step. Process variables and alternatives in this step include but are not limited to, reverse osmosis, nanofiltration, spray drying and combinations thereof. Products from stream 13a (retentate) include but are not limited to, water. Products from stream 13b (permeate) include but are not limited to, peptides, other proteins, and combinations thereof. Other proteins include but are not limited to lunasin, lectins, dehydrins, lipoxygenase, and combinations thereof.

Step 14 (as shown in FIG. 4B)—a protein fractionation step may be done by starting with soy whey protein, BBI, KTI, storage proteins, other proteins, and combinations thereof from streams 6a and/or 5a. Other proteins include but are not limited to lunasin, lectins, dehydrins, lipoxygenase, and combinations thereof. It includes an ultrafiltration (with pore sizes from 100 kD to 10 kD) step. Process variables and alternatives in this step include but are not limited to, crossflow membrane filtration, ultrafiltration, nanofiltration, and combinations thereof. Crossflow membrane filtration includes but is not limited to: spiral-wound, plate and frame, hollow fiber, ceramic, dynamic or rotating disk, nanofiber, and combinations thereof. The pH of step 14 can be between about 2.0 and about 12.0, preferably about 7.0. The temperature can be between about 5° C. and about 90° C., preferably about 75° C. Products from stream 14a (retentate) include but are not limited to, storage proteins. Products from stream 14b (permeate) include but are not limited to, soy whey protein, BBI, KTI and, other proteins. Other proteins include but are not limited to lunasin, lectins, dehydrins, lipoxygenase, and combinations thereof.

Step 15 (as shown in FIG. 4B)—a water removal step can start with soy whey protein, BBI, KTI and, other proteins from streams 6a, 5a, and/or 14b. Other proteins include but are not limited to lunasin, lectins, dehydrins, lipoxygenase, and combinations thereof. It includes an evaporation step. Process variables and alternatives in this step include but are not limited to, evaporation, nanofiltration, RO, and combinations thereof. Products from stream 15a (retentate) include but are not limited to, water. Stream 15b (permeate) products include but are not limited to soy whey protein, BBI, KTI and, other proteins. Other proteins include but are not limited to lunasin, lectins, dehydrins, lipoxygenase, and combinations thereof.

Step 16 (as shown in FIG. 4B)—a heat treatment and flash cooling step can start with soy whey protein, BBI, KTI and, other proteins from streams 6a, 5a, 14b, and/or 15b. Other proteins include but are not limited to lunasin, lectins, dehydrins, lipoxygenase, and combinations thereof. It includes an ultra high temperature step. Process variables and alternatives in this step include but are not limited to, heat sterilization, evaporation, and combinations thereof. Processing aids that can be used in this heat treatment and flash cooling step include but are not limited to, water, steam, and combinations thereof. The temperature can be between about 129° C. and about 160° C., preferably about 152° C. Temperature hold time can be between about 8 seconds and about 15 seconds, preferably about 9 seconds. Products from stream 16 include but are not limited to, soy whey protein.

Step 17 (as shown in FIG. 4B)—a drying step can start with soy whey protein, BBI, KTI and, other proteins from streams 6a, 5a, 14b, 15b, and/or 16. It includes a drying step. The liquid feed temperature can be between about 50° C. and about 95° C., preferably about 82° C. The inlet temperature can be between about 175° C. and about 370° C., preferably about 290° C. The exhaust temperature can be between about 65° C. and about 98° C., preferably about 88° C. Products from stream 17a (retentate) include but are not limited to, water. Products from stream 17b (permeate) include but are not limited to, soy whey protein which includes, BBI, KTI and, other proteins. Other proteins include but are not limited to lunasin, lectins, dehydrins, lipoxygenase, and combinations thereof.

The soy whey protein products of the current application include raw whey, a soy whey protein precursor after the ultrafiltration step of Step 17, a dry soy whey protein that can be dried by any means known in the art, and combinations thereof. All of these products can be used as is as soy whey protein or can be further processed to purify specific components of interest, such as, but not limited to BBI, KTI, and combinations thereof.

### IV. Preferred Embodiments of the Process for the Recovery of Soy Whey Protein

Embodiment 1 starts with Step 0 (See FIG. 4A) as follows: Whey protein pretreatment can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 can be between about 3.0 and about 6.0, preferably 4.5. The temperature can be between about 70° C. and about 95° C., preferably about 85° C. Temperature hold times can vary between about 0 minutes to about 20 minutes, preferably about 10 minutes. Products from the whey protein pretreatment include but are not limited to soluble components in the aqueous phase of the whey stream (pre-treated soy whey) (molecular weight of equal to or less than about 50 kDa in stream 0a (retentate) and insoluble large molecular weight proteins (between about 300 kDa and between about 50 kDa in stream 0b (permeate), such as pre-treated soy whey, storage proteins, and combinations thereof. Next.

Step 5 (See FIG. 4B) is done. Thus, the protein separation and concentration step in this embodiment starts with the whey from stream 0a. It includes an ultrafiltration step. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, preferably about 8.0. The temperature can be between about 5° C. and about 90° C., preferably about 75° C. Products from stream 5a (retentate) include but are not limited to, soy whey protein, BBI, KTI, storage proteins, other proteins and combinations thereof. Other proteins include but are not limited to lunasin, lectins, dehydrins, lipoxygenase, and combinations thereof. Products from stream 5b (permeate) include but are not limited to, peptides, soy oligosaccharides, minerals and combinations thereof. Soy oligosaccharides include but are not limited to sucrose, raffinose, stachyose, verbascose, monosaccharides, and combinations thereof. Minerals include but are not limited to calcium citrate.

Embodiment 2—starts with Step 0 (See FIG. 4A) as follows: Whey protein pretreatment can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 can be between about 3.0 and about 6.0, preferably 4.5. The temperature can be between about 70° C. and about 95° C., preferably about 85° C. Temperature hold times can vary between about 0 minutes to about 20 minutes, preferably about 10 minutes. Products from the whey protein pretreatment include but are not limited to soluble components in the aqueous phase of the whey stream (pre-treated soy whey) (molecular weight of equal to or less than about 50 kDa in stream 0a (retentate) and insoluble large molecular weight proteins (between about 300 kDa and between about 50 kDa in stream 0b (permeate), such as pre-treated soy whey, storage proteins, and combinations thereof.

Next Step 5 (See FIG. 4B) is done. Thus, the protein separation and concentration step in this embodiment starts with the whey from stream 0a. It includes an ultrafiltration step. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, preferably about 8.0. The temperature can be between about 5° C. and about 90° C., preferably about 75° C. Products from stream 5a (retentate) include but are not limited to, soy whey protein, BBI, KTI, storage proteins, other proteins and combinations thereof. Other proteins include but are not limited to lunasin, lectins, dehydrins, lipoxygenase, and combinations thereof. Products from stream 5b (permeate) include but are not limited to, peptides, soy oligosaccharides, minerals and combinations thereof. Soy oligosaccharides include but are not limited to sucrose, raffinose, stachyose, verbascose, monosaccharides, and combinations thereof. Minerals include but are not limited to calcium citrate.

Finally Step 6 (See FIG. 4B), the protein washing and purification step starts with soy whey protein, BBI, KTI, storage proteins, other proteins or purified pre-treated whey from stream 5a. It includes a diafiltration step. Process variables and alternatives in this step include but are not limited to, reslurrying, crossflow membrane filtration, ultrafiltration, water diafiltration, buffer diafiltration, and combinations thereof. Crossflow membrane filtration includes but is not limited to: spiral-wound, plate and frame, hollow fiber, ceramic, dynamic or rotating disk, nanofiber, and combinations thereof. Processing aids that can be used in the protein washing and purification step include but are not limited to, water, steam, and combinations thereof. The pH of step 6 can be between about 2.0 and about 12.0, preferably about 7.0. The temperature can be between about 5° C. and about 90° C., preferably about 75° C. Products from stream 6a (retentate) include but are not limited to, soy whey protein, BBI, KTI, storage proteins, other proteins, and combinations thereof. Other proteins include but are not limited to lunasin, lectins, dehydrins, lipoxygenase, and combinations thereof. Products from stream 6b (permeate) include but are not limited to, peptides, soy oligosaccharides, water, minerals, and combinations thereof. Soy oligosaccharides include but are not limited to sucrose, raffinose, stachyose, verbascose, monosaccharides, and combinations thereof. Minerals include but are not limited to calcium citrate.

Embodiment 3 starts with Step 0 (See FIG. 4A) which is a whey protein pretreatment that can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 can be between about 3.0 and about 6.0, preferably 4.5. The temperature can be between about 70° C. and about 95° C., preferably about 85° C. Temperature hold times can vary between about 0 minutes to about 20 minutes, preferably about 10 minutes. Products from the whey protein pretreatment include but are not limited to soluble components in the aqueous phase of the whey stream (pre-treated soy whey) (molecular weight of equal to or less than about 50 kDa in stream 0a (retentate) and insoluble large molecular weight proteins (between about 300 kDa and between about 50 kDa in stream 0b (permeate), such as pre-treated soy whey, storage proteins, and combinations thereof.

Step 3 (See FIG. 4A) the mineral precipitation step can start with purified pre-treated soy whey from stream 0a. It includes a precipitation step by pH and/or temperature change. Process variables and alternatives in this step include but are not limited to, an agitated or recirculating reaction tank. Processing aids that can be used in the mineral precipitation step include but are not limited to, acids, bases, calcium hydroxide, sodium hydroxide, hydrochloric acid, sodium chloride, phytase, and combinations thereof. The pH of step 3 can be between about 2.0 and about 12.0, preferably about 8.0. The temperature can be between about 5° C. and about 90° C., preferably about 50° C. The pH hold times can vary between about 0 minutes to about 60 minutes, preferably about 10 minutes. The product of stream 3 is a suspension of purified pre-treated soy whey and precipitated minerals.

Step 4 (See FIG. 4A) the mineral removal step can start with the suspension of purified pre-treated whey and precipitated minerals from stream 3. It includes a centrifugation step. Process variables and alternatives in this step include but are not limited to, centrifugation, filtration, dead-end filtration, crossflow membrane filtration and combinations thereof. Crossflow membrane filtration includes but is not limited to: spiral-wound, plate and frame, hollow fiber, ceramic, dynamic or rotating disk, nanofiber, and combinations thereof. Products from the mineral removal step include but are not limited to a de-mineralized pre-treated whey in stream 4a (retentate) and insoluble minerals with some protein mineral complexes in stream 4b (permeate).

Finally, Step 5 (See FIG. 4B) the protein separation and concentration step can start with purified pre-treated whey from stream 4a. It includes an ultrafiltration step. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, preferably about 8.0. The temperature can be between about 5° C. and about 90° C., preferably about 75° C. Products from stream 5a (retentate) include but are not limited to, soy whey protein, BBI, KTI, storage proteins, other proteins and combinations thereof. Other proteins include but are not limited to lunasin, lectins, dehydrins, lipoxygenase, and combinations thereof. Products from stream 5b (permeate) include but are not limited to, peptides, soy oligosaccharides, minerals and combinations thereof. Soy oligosaccharides include but are not limited to sucrose, raffinose, stachyose, verbascose, monosaccharides, and combinations thereof. Minerals include but are not limited to calcium citrate.

Embodiment 4 starts with Step 0 (See FIG. 4A) whey protein pretreatment that can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 can be between about 3.0 and about 6.0, preferably 4.5. The temperature can be between about 70° C. and about 95° C., preferably about 85° C. Temperature hold times can vary between about 0 minutes to about 20 minutes, preferably about 10 minutes. Products from the whey protein pretreatment include but are not limited to soluble components in the aqueous phase of the whey stream (pre-treated soy whey) (molecular weight of equal to or less than about 50 kDa in stream 0a (retentate) and insoluble large molecular weight proteins (between about 300 kDa and between about 50 kDa in stream 0b (permeate), such as pre-treated soy whey, storage proteins, and combinations thereof.

Step 3 (See FIG. 4A) the mineral precipitation step can start with purified pre-treated soy whey from stream 0a. It includes a precipitation step by pH and/or temperature change. Process variables and alternatives in this step include but are not limited to, an agitated or recirculating reaction tank. Processing aids that can be used in the mineral precipitation step include but are not limited to, acids, bases, calcium hydroxide, sodium hydroxide, hydrochloric acid, sodium chloride, phytase, and combinations thereof. The pH of step 3 can be between about 2.0 and about 12.0, preferably about 8.0. The temperature can be between about 5° C. and about 90° C., preferably about 50° C. The pH hold times can vary between about 0 minutes to about 60 minutes, preferably about 10 minutes. The product of stream 3 is a suspension of purified pre-treated soy whey and precipitated minerals.

Step 4 (See FIG. 4A)—the mineral removal step can start with the suspension of purified pre-treated whey and precipitated minerals from stream 3. It includes a centrifugation step. Process variables and alternatives in this step include but are not limited to, centrifugation, filtration, dead-end filtration, crossflow membrane filtration and combinations thereof. Crossflow membrane filtration includes but is not limited to: spiral-wound, plate and frame, hollow fiber, ceramic, dynamic or rotating disk, nanofiber, and combinations thereof. Products from the mineral removal step include but are not limited to a de-mineralized pre-treated whey in stream 4a (retentate) and insoluble minerals with some protein mineral complexes in stream 4b (permeate).

Step 5 (See FIG. 4B)—the protein separation and concentration step can start with purified pre-treated whey from stream 4a. It includes an ultrafiltration step. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, preferably about 8.0. The temperature can be between about 5° C. and about 90° C., preferably about 75° C. Products from stream 5a (retentate) include but are not limited to, soy whey protein, BBI, KTI, storage proteins, other proteins and combinations thereof. Other proteins include but are not limited to lunasin, lectins, dehydrins, lipoxygenase, and combinations thereof. Products from stream 5b (permeate) include but are not limited to, peptides, soy oligosaccharides, minerals and combinations thereof. Soy oligosaccharides include but are not limited to sucrose, raffinose, stachyose, verbascose, monosaccharides, and combinations thereof. Minerals include but are not limited to calcium citrate.

Finally, Step 6 (See FIG. 4B) the protein washing and purification step can start with soy whey protein, BBI, KTI, storage proteins, other proteins or purified pre-treated whey from stream 5a. It includes a diafiltration step. Process variables and alternatives in this step include but are not limited to, reslurrying, crossflow membrane filtration, ultrafiltration, water diafiltration, buffer diafiltration, and combinations thereof. Crossflow membrane filtration includes but is not limited to: spiral-wound, plate and frame, hollow fiber, ceramic, dynamic or rotating disk, nanofiber, and combinations thereof. Processing aids that can be used in the protein washing and purification step include but are not limited to, water, steam, and combinations thereof. The pH of step 6 can be between about 2.0 and about 12.0, preferably about 7.0. The temperature can be between about 5° C. and about 90° C., preferably about 75° C. Products from stream 6a (retentate) include but are not limited to, soy whey protein, BBI, KTI, storage proteins, other proteins, and combinations thereof. Other proteins include but are not limited to lunasin, lectins, dehydrins, lipoxygenase, and combinations thereof. Products from stream 6b (permeate) include but are not limited to, peptides, soy oligosaccharides, water, minerals, and combinations thereof. Soy oligosaccharides include but are not limited to sucrose, raffinose, stachyose, verbascose, monosaccharides, and combinations thereof. Minerals include but are not limited to calcium citrate.

Embodiment 5 starts with Step 0 (See FIG. 4A) the whey protein pretreatment can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 can be between about 3.0 and about 6.0, preferably 4.5. The temperature can be between about 70° C. and about 95° C., preferably about 85° C. Temperature hold times can vary between about 0 minutes to about 20 minutes, preferably about 10 minutes. Products from the whey protein pretreatment include but are not limited to soluble components in the aqueous phase of the whey stream (pre-treated soy whey) (molecular weight of equal to or less than about 50 kDa in stream 0a (retentate) and insoluble large molecular weight proteins (between about 300 kDa and between about 50 kDa in stream 0b (permeate), such as pre-treated soy whey, storage proteins, and combinations thereof.

Step 3 (See FIG. 4A) the mineral precipitation step can start with pre-treated soy whey from stream 0a. It includes a precipitation step by pH and/or temperature change. Process variables and alternatives in this step include but are not limited to, an agitated or recirculating reaction tank. Processing aids that can be used in the mineral precipitation step include but are not limited to, acids, bases, calcium hydroxide, sodium hydroxide, hydrochloric acid, sodium chloride, phytase, and combinations thereof. The pH of step 3 can be between about 2.0 and about 12.0, preferably about 8.0. The temperature can be between about 5° C. and about 90° C., preferably about 50° C. The pH hold times can vary between about 0 minutes to about 60 minutes, preferably about 10 minutes. The product of stream 3 is a suspension of purified pre-treated soy whey and precipitated minerals.

Step 4 (See FIG. 4A)—the mineral removal step can start with the suspension of purified pre-treated whey and precipitated minerals from stream 3. It includes a centrifugation step. Process variables and alternatives in this step include but are not limited to, centrifugation, filtration, dead-end filtration, crossflow membrane filtration and combinations thereof. Crossflow membrane filtration includes but is not limited to: spiral-wound, plate and frame, hollow fiber, ceramic, dynamic or rotating disk, nanofiber, and combinations thereof. Products from the mineral removal step include but are not limited to a de-mineralized pre-treated whey in stream 4a (retentate) and insoluble minerals with some protein mineral complexes in stream 4b (permeate).

Step 5 (See FIG. 4B) the protein separation and concentration step can start with purified pre-treated whey from stream 4a. It includes an ultrafiltration step. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, preferably about 8.0. The temperature can be between about 5° C. and about 90° C., preferably about 75° C. Products from stream 5a (retentate) include but are not limited to, soy whey protein, BBI, KTI, storage proteins, other proteins and combinations thereof. Other proteins include but are not limited to lunasin, lectins, dehydrins, lipoxygenase, and combinations thereof. Products from stream 5b (permeate) include but are not limited to, peptides, soy oligosaccharides, minerals and combinations thereof. Soy oligosaccharides include but are not limited to sucrose, raffinose, stachyose, verbascose, monosaccharides, and combinations thereof. Minerals include but are not limited to calcium citrate.

Step 6 (See FIG. 4B)—the protein washing and purification step can start with soy whey protein, BBI, KTI, storage proteins, other proteins or purified pre-treated whey from stream 5a. It includes a diafiltration step. Process variables and alternatives in this step include but are not limited to, reslurrying, crossflow membrane filtration, ultrafiltration, water diafiltration, buffer diafiltration, and combinations thereof. Crossflow membrane filtration includes but is not limited to: spiral-wound, plate and frame, hollow fiber, ceramic, dynamic or rotating disk, nanofiber, and combinations thereof. Processing aids that can be used in the protein washing and purification step include but are not limited to, water, steam, and combinations thereof. The pH of step 6 can be between about 2.0 and about 12.0, preferably about 7.0. The temperature can be between about 5° C. and about 90° C., preferably about 75° C. Products from stream 6a (retentate) include but are not limited to, soy whey protein, BBI, KTI, storage proteins, other proteins, and combinations thereof. Other proteins include but are not limited to lunasin, lectins, dehydrins, lipoxygenase, and combinations thereof. Products from stream 6b (permeate) include but are not limited to, peptides, soy oligosaccharides, water, minerals, and combinations thereof. Soy oligosaccharides include but are not limited to sucrose, raffinose, stachyose, verbascose, monosaccharides, and combinations thereof. Minerals include but are not limited to calcium citrate.

Step 16 (See FIG. 4B) a heat treatment and flash cooling step can start with soy whey protein, BBI, KTI and, other proteins from streams 6a. Other proteins include but are not limited to lunasin, lectins, dehydrins, lipoxygenase, and combinations thereof. It includes an ultra high temperature step. Process variables and alternatives in this step include but are not limited to, heat sterilization, evaporation, and combinations thereof. Processing aids that can be used in this heat treatment and flash cooling step include but are not limited to, water, steam, and combinations thereof. The temperature can be between about 129° C. and about 160° C., preferably about 152° C. Temperature hold time can be between about 8 seconds and about 15 seconds, preferably about 9 seconds. Products from stream 16 include but are not limited to, soy whey protein.

Finally, Step 17 (See FIG. 4B)—a drying step can start with soy whey protein , BBI, KTI and, other proteins from stream 16. It includes a drying step. The liquid feed temperature can be between about 50° C. and about 95° C., preferably about 82° C. The inlet temperature can be between about 175° C. and about 370° C., preferably about 290° C. The exhaust temperature can be between about 65° C. and about 98° C., preferably about 88° C. Products from stream 17a (retentate) include but are not limited to, water. Products from stream 17b (permeate) include but are not limited to, soy whey protein which includes, BBI, KTI and, other proteins. Other proteins include but are not limited to lunasin, lectins, dehydrins, lipoxygenase, and combinations thereof.

Embodiment 6 starts with Step 0 (See FIG. 4A) the whey protein pretreatment can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 can be between about 3.0 and about 6.0, preferably 4.5. The temperature can be between about 70° C. and about 95° C., preferably about 85° C. Temperature hold times can vary between about 0 minutes to about 20 minutes, preferably about 10 minutes. Products from the whey protein pretreatment include but are not limited to soluble components in the aqueous phase of the whey stream (pre-treated soy whey) (molecular weight of equal to or less than about 50 kDa in stream 0a (retentate) and insoluble large molecular weight proteins (between about 300 kDa and between about 50 kDa in stream 0b (permeate), such as pre-treated soy whey, storage proteins, and combinations thereof.

Step 3 (See FIG. 4A) the mineral precipitation step can start with pre-treated soy whey from stream 0a. It includes a precipitation step by pH and/or temperature change. Process variables and alternatives in this step include but are not limited to, an agitated or recirculating reaction tank. Processing aids that can be used in the mineral precipitation step include but are not limited to, acids, bases, calcium hydroxide, sodium hydroxide, hydrochloric acid, sodium chloride, phytase, and combinations thereof. The pH of step 3 can be between about 2.0 and about 12.0, preferably about 8.0. The temperature can be between about 5° C. and about 90° C., preferably about 50° C. The pH hold times can vary between about 0 minutes to about 60 minutes, preferably about 10 minutes. The product of stream 3 is a suspension of purified pre-treated soy whey and precipitated minerals.

Step 4 (See FIG. 4A) the mineral removal step can start with the suspension of purified pre-treated whey and precipitated minerals from stream 3. It includes a centrifugation step. Process variables and alternatives in this step include but are not limited to, centrifugation, filtration, dead-end filtration, crossflow membrane filtration and combinations thereof. Crossflow membrane filtration includes but is not limited to: spiral-wound, plate and frame, hollow fiber, ceramic, dynamic or rotating disk, nanofiber, and combinations thereof. Products from the mineral removal step include but are not limited to a de-mineralized pre-treated whey in stream 4a (retentate) and insoluble minerals with some protein mineral complexes in stream 4b (permeate).

Step 5 (See FIG. 4B) the protein separation and concentration step can start with purified pre-treated whey from stream 4a. It includes an ultrafiltration step. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, preferably about 8.0. The temperature can be between about 5° C. and about 90° C., preferably about 75° C. Products from stream 5a (retentate) include but are not limited to, soy whey protein, BBI, KTI, storage proteins, other proteins and combinations thereof. Other proteins include but are not limited to lunasin, lectins, dehydrins, lipoxygenase, and combinations thereof. Products from stream 5b (permeate) include but are not limited to, peptides, soy oligosaccharides, minerals and combinations thereof. Soy oligosaccharides include but are not limited to sucrose, raffinose, stachyose, verbascose, monosaccharides, and combinations thereof. Minerals include but are not limited to calcium citrate.

Step 6 (See FIG. 4B) the protein washing and purification step can start with soy whey protein, BBI, KTI, storage proteins, other proteins or purified pre-treated whey from stream 5a. It includes a diafiltration step. Process variables and alternatives in this step include but are not limited to, reslurrying, crossflow membrane filtration, ultrafiltration, water diafiltration, buffer diafiltration, and combinations thereof. Crossflow membrane filtration includes but is not limited to: spiral-wound, plate and frame, hollow fiber, ceramic, dynamic or rotating disk, nanofiber, and combinations thereof. Processing aids that can be used in the protein washing and purification step include but are not limited to, water, steam, and combinations thereof. The pH of step 6 can be between about 2.0 and about 12.0, preferably about 7.0. The temperature can be between about 5° C. and about 90° C., preferably about 75° C. Products from stream 6a (retentate) include but are not limited to, soy whey protein, BBI, KTI, storage proteins, other proteins, and combinations thereof. Other proteins include but are not limited to lunasin, lectins, dehydrins, lipoxygenase, and combinations thereof. Products from stream 6b (permeate) include but are not limited to, peptides, soy oligosaccharides, water, minerals, and combinations thereof. Soy oligosaccharides include but are not limited to sucrose, raffinose, stachyose, verbascose, monosaccharides, and combinations thereof. Minerals include but are not limited to calcium citrate.

Step 15 (See FIG. 4B) a water removal step can start with soy whey protein, BBI, KTI and, other proteins from stream 6a. Other proteins include but are not limited to lunasin, lectins, dehydrins, lipoxygenase, and combinations thereof. It includes an evaporation step. Process variables and alternatives in this step include but are not limited to, evaporation, nanofiltration, RO, and combinations thereof. Products from stream 15a (retentate) include but are not limited to, water. Stream 15b (permeate) products include but are not limited to soy whey protein, BBI, KTI and, other proteins. Other proteins include but are not limited to lunasin, lectins, dehydrins, lipoxygenase, and combinations thereof.

Step 16 (See FIG. 4B) a heat treatment and flash cooling step can start with soy whey protein, BBI, KTI and, other proteins from stream 15b. Other proteins include but are not limited to lunasin, lectins, dehydrins, lipoxygenase, and combinations thereof. It includes an ultra high temperature step. Process variables and alternatives in this step include but are not limited to, heat sterilization, evaporation, and combinations thereof. Processing aids that can be used in this heat treatment and flash cooling step include but are not limited to, water, steam, and combinations thereof. The temperature can be between about 129° C. and about 160° C., preferably about 152° C. Temperature hold time can be between about 8 seconds and about 15 seconds, preferably about 9 seconds. Products from stream 16 include but are not limited to, soy whey protein.

Finally, Step 17 (See FIG. 4B)—a drying step can start with soy whey protein , BBI, KTI and, other proteins from stream 16. It includes a drying step. The liquid feed temperature can be between about 50° C. and about 95° C., preferably about 82° C. The inlet temperature can be between about 175° C. and about 370° C., preferably about 290° C. The exhaust temperature can be between about 65° C. and about 98° C., preferably about 88° C. Products from stream 17a (retentate) include but are not limited to, water. Products from stream 17b (permeate) include but are not limited to, soy whey protein which includes, BBI, KTI and, other proteins. Other proteins include but are not limited to lunasin, lectins, dehydrins, lipoxygenase, and combinations thereof.

Embodiment 7 starts with Step 0 (See FIG. 4A) the whey protein pretreatment can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 can be between about 3.0 and about 6.0, preferably 4.5. The temperature can be between about 70° C. and about 95° C., preferably about 85° C. Temperature hold times can vary between about 0 minutes to about 20 minutes, preferably about 10 minutes. Products from the whey protein pretreatment include but are not limited to soluble components in the aqueous phase of the whey stream (pre-treated soy whey) (molecular weight of equal to or less than about 50 kDa in stream 0a (retentate) and insoluble large molecular weight proteins (between about 300 kDa and between about 50 kDa in stream 0b (permeate), such as pre-treated soy whey, storage proteins, and combinations thereof.

Step 2 (See FIG. 4A) a water and mineral removal can start with the pre-treated soy whey from stream 0b. It includes a nanofiltration step for water removal and partial mineral removal. Process variables and alternatives in this step include but are not limited to, crossflow membrane filtration, reverse osmosis, evaporation, nanofiltration, and combinations thereof. Crossflow membrane filtration includes but is not limited to: spiral-wound, plate and frame, hollow fiber, ceramic, dynamic or rotating disk, nanofiber, and combinations thereof. The pH of step 2 can be between about 2.0 and about 12.0, preferably about 5.3. The temperature can be between about 5° C. and about 90° C., preferably about 50° C. Products from this water removal step include but are not limited to purified pre-treated soy whey in stream 2a (retentate) and water, some minerals, monovalent cations and combinations thereof in stream 2b (permeate).

Finally, Step 5 (See FIG. 4B) the protein separation and concentration step can start with the whey from stream 2a. It includes an ultrafiltration step. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, preferably about 8.0. The temperature can be between about 5° C. and about 90° C., preferably about 75° C. Products from stream 5a (retentate) include but are not limited to, soy whey protein, BBI, KTI, storage proteins, other proteins and combinations thereof. Other proteins include but are not limited to lunasin, lectins, dehydrins, lipoxygenase, and combinations thereof. Products from stream 5b (permeate) include but are not limited to, peptides, soy oligosaccharides, minerals and combinations thereof. Soy oligosaccharides include but are not limited to sucrose, raffinose, stachyose, verbascose, monosaccharides, and combinations thereof. Minerals include but are not limited to calcium citrate.

Embodiment 8 starts with Step 0 (See FIG. 4A) the whey protein pretreatment can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 can be between about 3.0 and about 6.0, preferably 4.5. The temperature can be between about 70° C. and about 95° C., preferably about 85° C. Temperature hold times can vary between about 0 minutes to about 20 minutes, preferably about 10 minutes. Products from the whey protein pretreatment include but are not limited to soluble components in the aqueous phase of the whey stream (pre-treated soy whey) (molecular weight of equal to or less than about 50 kDa in stream 0a (retentate) and insoluble large molecular weight proteins (between about 300 kDa and between about 50 kDa in stream 0b (permeate), such as pre-treated soy whey, storage proteins, and combinations thereof.

Step 2 (See FIG. 4A) a water and mineral removal can start with the pre-treated soy whey from stream 0b. It includes a nanofiltration step for water removal and partial mineral removal. Process variables and alternatives in this step include but are not limited to, crossflow membrane filtration, reverse osmosis, evaporation, nanofiltration, and combinations thereof. Crossflow membrane filtration includes but is not limited to: spiral-wound, plate and frame, hollow fiber, ceramic, dynamic or rotating disk, nanofiber, and combinations thereof. The pH of step 2 can be between about 2.0 and about 12.0, preferably about 5.3. The temperature can be between about 5° C. and about 90° C., preferably about 50° C. Products from this water removal step include but are not limited to purified pre-treated soy whey in stream 2a (retentate) and water, some minerals, monovalent cations and combinations thereof in stream 2b (permeate).

Step 5 (See FIG. 4B) the protein separation and concentration step can start with the whey from stream 2a. It includes an ultrafiltration step. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, preferably about 8.0. The temperature can be between about 5° C. and about 90° C., preferably about 75° C. Products from stream 5a (retentate) include but are not limited to, soy whey protein, BBI, KTI, storage proteins, other proteins and combinations thereof. Other proteins include but are not limited to lunasin, lectins, dehydrins, lipoxygenase, and combinations thereof. Products from stream 5b (permeate) include but are not limited to, peptides, soy oligosaccharides, minerals and combinations thereof. Soy oligosaccharides include but are not limited to sucrose, raffinose, stachyose, verbascose, monosaccharides, and combinations thereof. Minerals include but are not limited to calcium citrate.

Finally, Step 6 (See FIG. 4B) the protein washing and purification step can start with soy whey protein, BBI, KTI, storage proteins, other proteins or purified pre-treated whey from stream 5a. It includes a diafiltration step. Process variables and alternatives in this step include but are not limited to, reslurrying, crossflow membrane filtration, ultrafiltration, water diafiltration, buffer diafiltration, and combinations thereof. Crossflow membrane filtration includes but is not limited to: spiral-wound, plate and frame, hollow fiber, ceramic, dynamic or rotating disk, nanofiber, and combinations thereof. Processing aids that can be used in the protein washing and purification step include but are not limited to, water, steam, and combinations thereof. The pH of step 6 can be between about 2.0 and about 12.0, preferably about 7.0. The temperature can be between about 5° C. and about 90° C., preferably about 75° C. Products from stream 6a (retentate) include but are not limited to, soy whey protein, BBI, KTI, storage proteins, other proteins, and combinations thereof. Other proteins include but are not limited to lunasin, lectins, dehydrins, lipoxygenase, and combinations thereof. Products from stream 6b (permeate) include but are not limited to, peptides, soy oligosaccharides, water, minerals, and combinations thereof. Soy oligosaccharides include but are not limited to sucrose, raffinose, stachyose, verbascose, monosaccharides, and combinations thereof. Minerals include but are not limited to calcium citrate.

Embodiment 9 starts with Step 0 (See FIG. 4A) the whey protein pretreatment can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 can be between about 3.0 and about 6.0, preferably 4.5. The temperature can be between about 70° C. and about 95° C., preferably about 85° C. Temperature hold times can vary between about 0 minutes to about 20 minutes, preferably about 10 minutes. Products from the whey protein pretreatment include but are not limited to soluble components in the aqueous phase of the whey stream (pre-treated soy whey) (molecular weight of equal to or less than about 50 kDa in stream 0a (retentate) and insoluble large molecular weight proteins (between about 300 kDa and between about 50 kDa in stream 0b (permeate), such as pre-treated soy whey, storage proteins, and combinations thereof.

Step 2 (See FIG. 4A) a water and mineral removal can start with the pre-treated soy whey from stream 0b. It includes a nanofiltration step for water removal and partial mineral removal. Process variables and alternatives in this step include but are not limited to, crossflow membrane filtration, reverse osmosis, evaporation, nanofiltration, and combinations thereof. Crossflow membrane filtration includes but is not limited to: spiral-wound, plate and frame, hollow fiber, ceramic, dynamic or rotating disk, nanofiber, and combinations thereof. The pH of step 2 can be between about 2.0 and about 12.0, preferably about 5.3. The temperature can be between about 5° C. and about 90° C., preferably about 50° C. Products from this water removal step include but are not limited to purified pre-treated soy whey in stream 2a (retentate) and water, some minerals, monovalent cations and combinations thereof in stream 2b (permeate).

Step 3 (See FIG. 4A) the mineral precipitation step can start with purified pre-treated soy whey from stream 2a . It includes a precipitation step by pH and/or temperature change. Process variables and alternatives in this step include but are not limited to, an agitated or recirculating reaction tank. Processing aids that can be used in the mineral precipitation step include but are not limited to, acids, bases, calcium hydroxide, sodium hydroxide, hydrochloric acid, sodium chloride, phytase, and combinations thereof. The pH of step 3 can be between about 2.0 and about 12.0, preferably about 8.0. The temperature can be between about 5° C. and about 90° C., preferably about 50° C. The pH hold times can vary between about 0 minutes to about 60 minutes, preferably about 10 minutes. The product of stream 3 is a suspension of purified pre-treated soy whey and precipitated minerals.

Step 4 (See FIG. 4A) the mineral removal step can start with the suspension of purified pre-treated whey and precipitated minerals from stream 3. It includes a centrifugation step. Process variables and alternatives in this step include but are not limited to, centrifugation, filtration, dead-end filtration, crossflow membrane filtration and combinations thereof. Crossflow membrane filtration includes but is not limited to: spiral-wound, plate and frame, hollow fiber, ceramic, dynamic or rotating disk, nanofiber, and combinations thereof. Products from the mineral removal step include but are not limited to a de-mineralized pre-treated whey in stream 4a (retentate) and insoluble minerals with some protein mineral complexes in stream 4b (permeate).

Step 5 (See FIG. 4B) the protein separation and concentration step can start with purified pre-treated whey from stream 4a. It includes an ultrafiltration step. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, preferably about 8.0. The temperature can be between about 5° C. and about 90° C., preferably about 75° C. Products from stream 5a (retentate) include but are not limited to, soy whey protein, BBI, KTI, storage proteins, other proteins and combinations thereof. Other proteins include but are not limited to lunasin, lectins, dehydrins, lipoxygenase, and combinations thereof. Products from stream 5b (permeate) include but are not limited to, peptides, soy oligosaccharides, minerals and combinations thereof. Soy oligosaccharides include but are not limited to sucrose, raffinose, stachyose, verbascose, monosaccharides, and combinations thereof. Minerals include but are not limited to calcium citrate.

Embodiment 10 starts with Step 0 (See FIG. 4A) the whey protein pretreatment can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 can be between about 3.0 and about 6.0, preferably 4.5. The temperature can be between about 70° C. and about 95° C., preferably about 85° C. Temperature hold times can vary between about 0 minutes to about 20 minutes, preferably about 10 minutes. Products from the whey protein pretreatment include but are not limited to soluble components in the aqueous phase of the whey stream (pre-treated soy whey) (molecular weight of equal to or less than about 50 kDa in stream 0a (retentate) and insoluble large molecular weight proteins (between about 300 kDa and between about 50 kDa in stream 0b (permeate), such as pre-treated soy whey, storage proteins, and combinations thereof.

Step 2 (See FIG. 4A) a water and mineral removal can start with the pre-treated soy whey from stream 0b. It includes a nanofiltration step for water removal and partial mineral removal. Process variables and alternatives in this step include but are not limited to, crossflow membrane filtration, reverse osmosis, evaporation, nanofiltration, and combinations thereof. Crossflow membrane filtration includes but is not limited to: spiral-wound, plate and frame, hollow fiber, ceramic, dynamic or rotating disk, nanofiber, and combinations thereof. The pH of step 2 can be between about 2.0 and about 12.0, preferably about 5.3. The temperature can be between about 5° C. and about 90° C., preferably about 50° C. Products from this water removal step include but are not limited to purified pre-treated soy whey in stream 2a (retentate) and water, some minerals, monovalent cations and combinations thereof in stream 2b (permeate).

Step 3 (See FIG. 4A) the mineral precipitation step can start with purified pre-treated soy whey from stream 2a. It includes a precipitation step by pH and/or temperature change. Process variables and alternatives in this step include but are not limited to, an agitated or recirculating reaction tank. Processing aids that can be used in the mineral precipitation step include but are not limited to, acids, bases, calcium hydroxide, sodium hydroxide, hydrochloric acid, sodium chloride, phytase, and combinations thereof. The pH of step 3 can be between about 2.0 and about 12.0, preferably about 8.0. The temperature can be between about 5° C. and about 90° C., preferably about 50° C. The pH hold times can vary between about 0 minutes to about 60 minutes, preferably about 10 minutes. The product of stream 3 is a suspension of purified pre-treated soy whey and precipitated minerals.

Step 4 (See FIG. 4A) the mineral removal step can start with the suspension of purified pre-treated whey and precipitated minerals from stream 3. It includes a centrifugation step. Process variables and alternatives in this step include but are not limited to, centrifugation, filtration, dead-end filtration, crossflow membrane filtration and combinations thereof. Crossflow membrane filtration includes but is not limited to: spiral-wound, plate and frame, hollow fiber, ceramic, dynamic or rotating disk, nanofiber, and combinations thereof. Products from the mineral removal step include but are not limited to a de-mineralized pre-treated whey in stream 4a (retentate) and insoluble minerals with some protein mineral complexes in stream 4b (permeate).

Step 5 (See FIG. 4B) the protein separation and concentration step can start with purified pre-treated whey from stream 4a. It includes an ultrafiltration step. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, preferably about 8.0. The temperature can be between about 5° C. and about 90° C., preferably about 75° C. Products from stream 5a (retentate) include but are not limited to, soy whey protein, BBI, KTI, storage proteins, other proteins and combinations thereof. Other proteins include but are not limited to lunasin, lectins, dehydrins, lipoxygenase, and combinations thereof. Products from stream 5b (permeate) include but are not limited to, peptides, soy oligosaccharides, minerals and combinations thereof. Soy oligosaccharides include but are not limited to sucrose, raffinose, stachyose, verbascose, monosaccharides, and combinations thereof. Minerals include but are not limited to calcium citrate.

Finally, Step 6 (See FIG. 4B) the protein washing and purification step can start with soy whey protein, BBI, KTI, storage proteins, other proteins or purified pre-treated whey from stream 5a. It includes a diafiltration step. Process variables and alternatives in this step include but are not limited to, reslurrying, crossflow membrane filtration, ultrafiltration, water diafiltration, buffer diafiltration, and combinations thereof. Crossflow membrane filtration includes but is not limited to: spiral-wound, plate and frame, hollow fiber, ceramic, dynamic or rotating disk, nanofiber, and combinations thereof. Processing aids that can be used in the protein washing and purification step include but are not limited to, water, steam, and combinations thereof. The pH of step 6 can be between about 2.0 and about 12.0, preferably about 7.0. The temperature can be between about 5° C. and about 90° C., preferably about 75° C. Products from stream 6a (retentate) include but are not limited to, soy whey protein, BBI, KTI, storage proteins, other proteins, and combinations thereof. Other proteins include but are not limited to lunasin, lectins, dehydrins, lipoxygenase, and combinations thereof. Products from stream 6b (permeate) include but are not limited to, peptides, soy oligosaccharides, water, minerals, and combinations thereof. Soy oligosaccharides include but are not limited to sucrose, raffinose, stachyose, verbascose, monosaccharides, and combinations thereof. Minerals include but are not limited to calcium citrate.

Embodiment 11 starts with Step 0 (See FIG. 4A) the whey protein pretreatment can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 can be between about 3.0 and about 6.0, preferably 4.5. The temperature can be between about 70° C. and about 95° C., preferably about 85° C. Temperature hold times can vary between about 0 minutes to about 20 minutes, preferably about 10 minutes. Products from the whey protein pretreatment include but are not limited to soluble components in the aqueous phase of the whey stream (pre-treated soy whey) (molecular weight of equal to or less than about 50 kDa in stream 0a (retentate) and insoluble large molecular weight proteins (between about 300 kDa and between about 50 kDa in stream 0b (permeate), such as pre-treated soy whey, storage proteins, and combinations thereof.

Step 2 (See FIG. 4A) a water and mineral removal can start with the pre-treated soy whey from stream 0b. It includes a nanofiltration step for water removal and partial mineral removal. Process variables and alternatives in this step include but are not limited to, crossflow membrane filtration, reverse osmosis, evaporation, nanofiltration, and combinations thereof. Crossflow membrane filtration includes but is not limited to: spiral-wound, plate and frame, hollow fiber, ceramic, dynamic or rotating disk, nanofiber, and combinations thereof. The pH of step 2 can be between about 2.0 and about 12.0, preferably about 5.3. The temperature can be between about 5° C. and about 90° C., preferably about 50° C. Products from this water removal step include but are not limited to purified pre-treated soy whey in stream 2a (retentate) and water, some minerals, monovalent cations and combinations thereof in stream 2b (permeate).

Step 3 (See FIG. 4A) the mineral precipitation step can start with purified pre-treated soy whey from stream 2a. It includes a precipitation step by pH and/or temperature change. Process variables and alternatives in this step include but are not limited to, an agitated or recirculating reaction tank. Processing aids that can be used in the mineral precipitation step include but are not limited to, acids, bases, calcium hydroxide, sodium hydroxide, hydrochloric acid, sodium chloride, phytase, and combinations thereof. The pH of step 3 can be between about 2.0 and about 12.0, preferably about 8.0. The temperature can be between about 5° C. and about 90° C., preferably about 50° C. The pH hold times can vary between about 0 minutes to about 60 minutes, preferably about 10 minutes. The product of stream 3 is a suspension of purified pre-treated soy whey and precipitated minerals.

Step 4 (See FIG. 4A)—the mineral removal step can start with the suspension of purified pre-treated whey and precipitated minerals from stream 3. It includes a centrifugation step. Process variables and alternatives in this step include but are not limited to, centrifugation, filtration, dead-end filtration, crossflow membrane filtration and combinations thereof. Crossflow membrane filtration includes but is not limited to: spiral-wound, plate and frame, hollow fiber, ceramic, dynamic or rotating disk, nanofiber, and combinations thereof. Products from the mineral removal step include but are not limited to a de-mineralized pre-treated whey in stream 4a (retentate) and insoluble minerals with some protein mineral complexes in stream 4b (permeate).

Step 5 (See FIG. 4B)—the protein separation and concentration step can start with purified pre-treated whey from stream 4a. It includes an ultrafiltration step. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, preferably about 8.0. The temperature can be between about 5° C. and about 90° C., preferably about 75° C. Products from stream 5a (retentate) include but are not limited to, soy whey protein, BBI, KTI, storage proteins, other proteins and combinations thereof. Other proteins include but are not limited to lunasin, lectins, dehydrins, lipoxygenase, and combinations thereof. Products from stream 5b (permeate) include but are not limited to, peptides, soy oligosaccharides, minerals and combinations thereof. Soy oligosaccharides include but are not limited to sucrose, raffinose, stachyose, verbascose, monosaccharides, and combinations thereof. Minerals include but are not limited to calcium citrate.

Step 6 (See FIG. 4B) the protein washing and purification step can start with soy whey protein, BBI, KTI, storage proteins, other proteins or purified pre-treated whey from stream 5a. It includes a diafiltration step. Process variables and alternatives in this step include but are not limited to, reslurrying, crossflow membrane filtration, ultrafiltration, water diafiltration, buffer diafiltration, and combinations thereof. Crossflow membrane filtration includes but is not limited to: spiral-wound, plate and frame, hollow fiber, ceramic, dynamic or rotating disk, nanofiber, and combinations thereof. Processing aids that can be used in the protein washing and purification step include but are not limited to, water, steam, and combinations thereof. The pH of step 6 can be between about 2.0 and about 12.0, preferably about 7.0. The temperature can be between about 5° C. and about 90° C., preferably about 75° C. Products from stream 6a (retentate) include but are not limited to, soy whey protein, BBI, KTI, storage proteins, other proteins, and combinations thereof. Other proteins include but are not limited to lunasin, lectins, dehydrins, lipoxygenase, and combinations thereof. Products from stream 6b (permeate) include but are not limited to, peptides, soy oligosaccharides, water, minerals, and combinations thereof. Soy oligosaccharides include but are not limited to sucrose, raffinose, stachyose, verbascose, monosaccharides, and combinations thereof. Minerals include but are not limited to calcium citrate.

Step 16 (See FIG. 4B) a heat treatment and flash cooling step can start with soy whey protein, BBI, KTI and, other proteins from stream 6a. Other proteins include but are not limited to lunasin, lectins, dehydrins, lipoxygenase, and combinations thereof. It includes an ultra high temperature step. Process variables and alternatives in this step include but are not limited to, heat sterilization, evaporation, and combinations thereof. Processing aids that can be used in this heat treatment and flash cooling step include but are not limited to, water, steam, and combinations thereof. The temperature can be between about 129° C. and about 160° C., preferably about 152° C. Temperature hold time can be between about 8 seconds and about 15 seconds, preferably about 9 seconds. Products from stream 16 include but are not limited to, soy whey protein.

Finally, Step 17 (See FIG. 4B)—a drying step can start with soy whey protein, BBI, KTI and, other proteins from stream 16. It includes a drying step. The liquid feed temperature can be between about 50° C. and about 95° C., preferably about 82° C. The inlet temperature can be between about 175° C. and about 370° C., preferably about 290° C. The exhaust temperature can be between about 65° C. and about 98° C., preferably about 88° C. Products from stream 17a (retentate) include but are not limited to, water. Products from stream 17b (permeate) include but are not limited to, soy whey protein which includes, BBI, KTI and, other proteins. Other proteins include but are not limited to lunasin, lectins, dehydrins, lipoxygenase, and combinations thereof.

Embodiment 12 starts with Step 0 (See FIG. 4A) the whey protein pretreatment can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 can be between about 3.0 and about 6.0, preferably 4.5. The temperature can be between about 70° C. and about 95° C., preferably about 85° C. Temperature hold times can vary between about 0 minutes to about 20 minutes, preferably about 10 minutes. Products from the whey protein pretreatment include but are not limited to soluble components in the aqueous phase of the whey stream (pre-treated soy whey) (molecular weight of equal to or less than about 50 kDa in stream 0a (retentate) and insoluble large molecular weight proteins (between about 300 kDa and between about 50 kDa in stream 0b (permeate), such as pre-treated soy whey, storage proteins, and combinations thereof.

Step 2 (See FIG. 4A) a water and mineral removal can start with the purified pre-treated soy whey from stream 1b or pre-treated soy whey from stream 0b. It includes a nanofiltration step for water removal and partial mineral removal. Process variables and alternatives in this step include but are not limited to, crossflow membrane filtration, reverse osmosis, evaporation, nanofiltration, and combinations thereof. Crossflow membrane filtration includes but is not limited to: spiral-wound, plate and frame, hollow fiber, ceramic, dynamic or rotating disk, nanofiber, and combinations thereof. The pH of step 2 can be between about 2.0 and about 12.0, preferably about 5.3. The temperature can be between about 5° C. and about 90° C., preferably about 50° C. Products from this water removal step include but are not limited to purified pre-treated soy whey in stream 2a (retentate) and water, some minerals, monovalent cations and combinations thereof in stream 2b (permeate).

Step 3 (See FIG. 4A) the mineral precipitation step can start with purified pre-treated soy whey from stream 2a. It includes a precipitation step by pH and/or temperature change. Process variables and alternatives in this step include but are not limited to, an agitated or recirculating reaction tank. Processing aids that can be used in the mineral precipitation step include but are not limited to, acids, bases, calcium hydroxide, sodium hydroxide, hydrochloric acid, sodium chloride, phytase, and combinations thereof. The pH of step 3 can be between about 2.0 and about 12.0, preferably about 8.0. The temperature can be between about 5° C. and about 90° C., preferably about 50° C. The pH hold times can vary between about 0 minutes to about 60 minutes, preferably about 10 minutes. The product of stream 3 is a suspension of purified pre-treated soy whey and precipitated minerals.

Step 4 (See FIG. 4A) the mineral removal step can start with the suspension of purified pre-treated whey and precipitated minerals from stream 3. It includes a centrifugation step. Process variables and alternatives in this step include but are not limited to, centrifugation, filtration, dead-end filtration, crossflow membrane filtration and combinations thereof. Crossflow membrane filtration includes but is not limited to: spiral-wound, plate and frame, hollow fiber, ceramic, dynamic or rotating disk, nanofiber, and combinations thereof. Products from the mineral removal step include but are not limited to a de-mineralized pre-treated whey in stream 4a (retentate) and insoluble minerals with some protein mineral complexes in stream 4b (permeate).

Step 5 (See FIG. 4B) the protein separation and concentration step can start with purified pre-treated whey from stream 4a. It includes an ultrafiltration step. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, preferably about 8.0. The temperature can be between about 5° C. and about 90° C., preferably about 75° C. Products from stream 5a (retentate) include but are not limited to, soy whey protein, BBI, KTI, storage proteins, other proteins and combinations thereof. Other proteins include but are not limited to lunasin, lectins, dehydrins, lipoxygenase, and combinations thereof. Products from stream 5b (permeate) include but are not limited to, peptides, soy oligosaccharides, minerals and combinations thereof. Soy oligosaccharides include but are not limited to sucrose, raffinose, stachyose, verbascose, monosaccharides, and combinations thereof. Minerals include but are not limited to calcium citrate.

Step 6 (See FIG. 4B) the protein washing and purification step can start with soy whey protein, BBI, KTI, storage proteins, other proteins or purified pre-treated whey from stream 5a. It includes a diafiltration step. Process variables and alternatives in this step include but are not limited to, reslurrying, crossflow membrane filtration, ultrafiltration, water diafiltration, buffer diafiltration, and combinations thereof. Crossflow membrane filtration includes but is not limited to: spiral-wound, plate and frame, hollow fiber, ceramic, dynamic or rotating disk, nanofiber, and combinations thereof. Processing aids that can be used in the protein washing and purification step include but are not limited to, water, steam, and combinations thereof. The pH of step 6 can be between about 2.0 and about 12.0, preferably about 7.0. The temperature can be between about 5° C. and about 90° C., preferably about 75° C. Products from stream 6a (retentate) include but are not limited to, soy whey protein, BBI, KTI, storage proteins, other proteins, and combinations thereof. Other proteins include but are not limited to lunasin, lectins, dehydrins, lipoxygenase, and combinations thereof. Products from stream 6b (permeate) include but are not limited to, peptides, soy oligosaccharides, water, minerals, and combinations thereof. Soy oligosaccharides include but are not limited to sucrose, raffinose, stachyose, verbascose, monosaccharides, and combinations thereof. Minerals include but are not limited to calcium citrate.

Step 15 (See FIG. 4B) a water removal step can start with soy whey protein, BBI, KTI and, other proteins from stream 6a. Other proteins include but are not limited to lunasin, lectins, dehydrins, lipoxygenase, and combinations thereof. It includes an evaporation step. Process variables and alternatives in this step include but are not limited to, evaporation, nanofiltration, RO, and combinations thereof. Products from stream 15a (retentate) include but are not limited to, water. Stream 15b (permeate) products include but are not limited to soy whey protein, BBI, KTI and, other proteins. Other proteins include but are not limited to lunasin, lectins, dehydrins, lipoxygenase, and combinations thereof.

Step 16 (See FIG. 4B) a heat treatment and flash cooling step can start with soy whey protein, BBI, KTI and, other proteins from stream 15b. Other proteins include but are not limited to lunasin, lectins, dehydrins, lipoxygenase, and combinations thereof. It includes an ultra high temperature step. Process variables and alternatives in this step include but are not limited to, heat sterilization, evaporation, and combinations thereof. Processing aids that can be used in this heat treatment and flash cooling step include but are not limited to, water, steam, and combinations thereof. The temperature can be between about 129° C. and about 160° C., preferably about 152° C. Temperature hold time can be between about 8 seconds and about 15 seconds, preferably about 9 seconds. Products from stream 16 include but are not limited to, soy whey protein.

Finally, Step 17 (See FIG. 4B) a drying step can start with soy whey protein, BBI, KTI and, other proteins from stream 16. It includes a drying step. The liquid feed temperature can be between about 50° C. and about 95° C., preferably about 82° C. The inlet temperature can be between about 175° C. and about 370° C., preferably about 290° C. The exhaust temperature can be between about 65° C. and about 98° C., preferably about 88° C. Products from stream 17a (retentate) include but are not limited to, water. Products from stream 17b (permeate) include but are not limited to, soy whey protein which includes, BBI, KTI and, other proteins. Other proteins include but are not limited to lunasin, lectins, dehydrins, lipoxygenase, and combinations thereof.

Embodiment 13 starts with Step 0 (See FIG. 4A) the whey protein pretreatment can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 can be between about 3.0 and about 6.0, preferably 4.5. The temperature can be between about 70° C. and about 95° C., preferably about 85° C. Temperature hold times can vary between about 0 minutes to about 20 minutes, preferably about 10 minutes. Products from the whey protein pretreatment include but are not limited to soluble components in the aqueous phase of the whey stream (pre-treated soy whey) (molecular weight of equal to or less than about 50 kDa in stream 0a (retentate) and insoluble large molecular weight proteins (between about 300 kDa and between about 50 kDa in stream 0b (permeate), such as pre-treated soy whey, storage proteins, and combinations thereof.

Step 3 (See FIG. 4A) the mineral precipitation step can start with pre-treated soy whey from stream 0a. It includes a precipitation step by pH and/or temperature change. Process variables and alternatives in this step include but are not limited to, an agitated or recirculating reaction tank. Processing aids that can be used in the mineral precipitation step include but are not limited to, acids, bases, calcium hydroxide, sodium hydroxide, hydrochloric acid, sodium chloride, phytase, and combinations thereof. The pH of step 3 can be between about 2.0 and about 12.0, preferably about 8.0. The temperature can be between about 5° C. and about 90° C., preferably about 50° C. The pH hold times can vary between about 0 minutes to about 60 minutes, preferably about 10 minutes. The product of stream 3 is a suspension of purified pre-treated soy whey and precipitated minerals.

Step 4 (See FIG. 4A) the mineral removal step can start with the suspension of purified pre-treated whey and precipitated minerals from stream 3. It includes a centrifugation step. Process variables and alternatives in this step include but are not limited to, centrifugation, filtration, dead-end filtration, crossflow membrane filtration and combinations thereof. Crossflow membrane filtration includes but is not limited to: spiral-wound, plate and frame, hollow fiber, ceramic, dynamic or rotating disk, nanofiber, and combinations thereof. Products from the mineral removal step include but are not limited to a de-mineralized pre-treated whey in stream 4a (retentate) and insoluble minerals with some protein mineral complexes in stream 4b (permeate).

Step 2 (See FIG. 4A) a water and mineral removal can start with the purified pre-treated soy whey from stream 1b or pre-treated soy whey from stream 0b. It includes a nanofiltration step for water removal and partial mineral removal. Process variables and alternatives in this step include but are not limited to, crossflow membrane filtration, reverse osmosis, evaporation, nanofiltration, and combinations thereof. Crossflow membrane filtration includes but is not limited to: spiral-wound, plate and frame, hollow fiber, ceramic, dynamic or rotating disk, nanofiber, and combinations thereof. The pH of step 2 can be between about 2.0 and about 12.0, preferably about 5.3. The temperature can be between about 5° C. and about 90° C., preferably about 50° C. Products from this water removal step include but are not limited to purified pre-treated soy whey in stream 2a (retentate) and water, some minerals, monovalent cations and combinations thereof in stream 2b (permeate).

Finally, Step 5 (See FIG. 4B) the protein separation and concentration step can start with the whey from stream 2a. It includes an ultrafiltration step. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, preferably about 8.0. The temperature can be between about 5° C. and about 90° C., preferably about 75° C. Products from stream 5a(retentate) include but are not limited to, soy whey protein, BBI, KTI, storage proteins, other proteins and combinations thereof. Other proteins include but are not limited to lunasin, lectins, dehydrins, lipoxygenase, and combinations thereof. Products from stream 5b (permeate) include but are not limited to, peptides, soy oligosaccharides, minerals and combinations thereof. Soy oligosaccharides include but are not limited to sucrose, raffinose, stachyose, verbascose, monosaccharides, and combinations thereof. Minerals include but are not limited to calcium citrate.

Embodiment 14 starts with Step 0 (See FIG. 4A) the whey protein pretreatment can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 can be between about 3.0 and about 6.0, preferably 4.5. The temperature can be between about 70° C. and about 95° C., preferably about 85° C. Temperature hold times can vary between about 0 minutes to about 20 minutes, preferably about 10 minutes. Products from the whey protein pretreatment include but are not limited to soluble components in the aqueous phase of the whey stream (pre-treated soy whey) (molecular weight of equal to or less than about 50 kDa in stream 0a (retentate) and insoluble large molecular weight proteins (between about 300 kDa and between about 50 kDa in stream 0b (permeate), such as pre-treated soy whey, storage proteins, and combinations thereof.

Step 3 (See FIG. 4A) the mineral precipitation step can start with pretreated soy whey from stream 0a. It includes a precipitation step by pH and/or temperature change. Process variables and alternatives in this step include but are not limited to, an agitated or recirculating reaction tank. Processing aids that can be used in the mineral precipitation step include but are not limited to, acids, bases, calcium hydroxide, sodium hydroxide, hydrochloric acid, sodium chloride, phytase, and combinations thereof. The pH of step 3 can be between about 2.0 and about 12.0, preferably about 8.0. The temperature can be between about 5° C. and about 90° C., preferably about 50° C. The pH hold times can vary between about 0 minutes to about 60 minutes, preferably about 10 minutes. The product of stream 3 is a suspension of purified pre-treated soy whey and precipitated minerals.

Step 4 (See FIG. 4A) the mineral removal step can start with the suspension of purified pre-treated whey and precipitated minerals from stream 3. It includes a centrifugation step. Process variables and alternatives in this step include but are not limited to, centrifugation, filtration, dead-end filtration, crossflow membrane filtration and combinations thereof. Crossflow membrane filtration includes but is not limited to: spiral-wound, plate and frame, hollow fiber, ceramic, dynamic or rotating disk, nanofiber, and combinations thereof. Products from the mineral removal step include but are not limited to a de-mineralized pre-treated whey in stream 4a (retentate) and insoluble minerals with some protein mineral complexes in stream 4b (permeate).

Step 2 (See FIG. 4A) a water and mineral removal can start with the purified pre-treated soy whey from stream 4a. It includes a nanofiltration step for water removal and partial mineral removal. Process variables and alternatives in this step include but are not limited to, crossflow membrane filtration, reverse osmosis, evaporation, nanofiltration, and combinations thereof. Crossflow membrane filtration includes but is not limited to: spiral-wound, plate and frame, hollow fiber, ceramic, dynamic or rotating disk, nanofiber, and combinations thereof. The pH of step 2 can be between about 2.0 and about 12.0, preferably about 5.3. The temperature can be between about 5° C. and about 90° C., preferably about 50° C. Products from this water removal step include but are not limited to purified pre-treated soy whey in stream 2a retentate) and water, some minerals, monovalent cations and combinations thereof in stream 2b permeate).

Step 5 (See FIG. 4B) the protein separation and concentration step can start with the whey from stream 2a. It includes an ultrafiltration step. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, preferably about 8.0. The temperature can be between about 5° C. and about 90° C., preferably about 75° C. Products from stream 5a (retentate) include but are not limited to, soy whey protein, BBI, KTI, storage proteins, other proteins and combinations thereof. Other proteins include but are not limited to lunasin, lectins, dehydrins, lipoxygenase, and combinations thereof. Products from stream 5b (permeate) include but are not limited to, peptides, soy oligosaccharides, minerals and combinations thereof. Soy oligosaccharides include but are not limited to sucrose, raffinose, stachyose, verbascose, monosaccharides, and combinations thereof. Minerals include but are not limited to calcium citrate.

Finally, Step 6 (See FIG. 4B) the protein washing and purification step can start with soy whey protein, BBI, KTI, storage proteins, other proteins or purified pre-treated whey from stream 5a. It includes a diafiltration step. Process variables and alternatives in this step include but are not limited to, reslurrying, crossflow membrane filtration, ultrafiltration, water diafiltration, buffer diafiltration, and combinations thereof. Crossflow membrane filtration includes but is not limited to: spiral-wound, plate and frame, hollow fiber, ceramic, dynamic or rotating disk, nanofiber, and combinations thereof. Processing aids that can be used in the protein washing and purification step include but are not limited to, water, steam, and combinations thereof. The pH of step 6 can be between about 2.0 and about 12.0, preferably about 7.0. The temperature can be between about 5° C. and about 90° C., preferably about 75° C. Products from stream 6a (retentate) include but are not limited to, soy whey protein, BBI, KTI, storage proteins, other proteins, and combinations thereof. Other proteins include but are not limited to lunasin, lectins, dehydrins, lipoxygenase, and combinations thereof. Products from stream 6b (permeate) include but are not limited to, peptides, soy oligosaccharides, water, minerals, and combinations thereof. Soy oligosaccharides include but are not limited to sucrose, raffinose, stachyose, verbascose, monosaccharides, and combinations thereof. Minerals include but are not limited to calcium citrate.

Embodiment 15 starts with Step 0 (See FIG. 4A) the whey protein pretreatment can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 can be between about 3.0 and about 6.0, preferably 4.5. The temperature can be between about 70° C. and about 95° C., preferably about 85° C. Temperature hold times can vary between about 0 minutes to about 20 minutes, preferably about 10 minutes. Products from the whey protein pretreatment include but are not limited to soluble components in the aqueous phase of the whey stream (pre-treated soy whey) (molecular weight of equal to or less than about 50 kDa in stream 0a (retentate) and insoluble large molecular weight proteins (between about 300 kDa and between about 50 kDa in stream 0b (permeate), such as pre-treated soy whey, storage proteins, and combinations thereof.

Step 3 (See FIG. 4A) the mineral precipitation step can start with pretreated soy whey from stream 0a. It includes a precipitation step by pH and/or temperature change. Process variables and alternatives in this step include but are not limited to, an agitated or recirculating reaction tank. Processing aids that can be used in the mineral precipitation step include but are not limited to, acids, bases, calcium hydroxide, sodium hydroxide, hydrochloric acid, sodium chloride, phytase, and combinations thereof. The pH of step 3 can be between about 2.0 and about 12.0, preferably about 8.0. The temperature can be between about 5° C. and about 90° C., preferably about 50° C. The pH hold times can vary between about 0 minutes to about 60 minutes, preferably about 10 minutes. The product of stream 3 is a suspension of purified pre-treated soy whey and precipitated minerals.

Step 4 (See FIG. 4A) the mineral removal step can start with the suspension of purified pre-treated whey and precipitated minerals from stream 3. It includes a centrifugation step. Process variables and alternatives in this step include but are not limited to, centrifugation, filtration, dead-end filtration, crossflow membrane filtration and combinations thereof. Crossflow membrane filtration includes but is not limited to: spiral-wound, plate and frame, hollow fiber, ceramic, dynamic or rotating disk, nanofiber, and combinations thereof. Products from the mineral removal step include but are not limited to a de-mineralized pre-treated whey in stream 4a (retentate) and insoluble minerals with some protein mineral complexes in stream 4b (permeate).

Step 2 (See FIG. 4A) a water and mineral removal can start with the purified pre-treated soy whey from stream 1b or pre-treated soy whey from stream 0b. It includes a nanofiltration step for water removal and partial mineral removal. Process variables and alternatives in this step include but are not limited to, crossflow membrane filtration, reverse osmosis, evaporation, nanofiltration, and combinations thereof. Crossflow membrane filtration includes but is not limited to: spiral-wound, plate and frame, hollow fiber, ceramic, dynamic or rotating disk, nanofiber, and combinations thereof. The pH of step 2 can be between about 2.0 and about 12.0, preferably about 5.3. The temperature can be between about 5° C. and about 90° C., preferably about 50° C. Products from this water removal step include but are not limited to purified pre-treated soy whey in stream 2a (retentate) and water, some minerals, monovalent cations and combinations thereof in stream 2b (permeate).

Step 5 (See FIG. 4B) the protein separation and concentration step can start with the whey from stream 2a. It includes an ultrafiltration step. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, preferably about 8.0. The temperature can be between about 5° C. and about 90° C., preferably about 75° C. Products from stream 5a (retentate) include but are not limited to, soy whey protein, BBI, KTI, storage proteins, other proteins and combinations thereof. Other proteins include but are not limited to lunasin, lectins, dehydrins, lipoxygenase, and combinations thereof. Products from stream 5b (permeate) include but are not limited to, peptides, soy oligosaccharides, minerals and combinations thereof. Soy oligosaccharides include but are not limited to sucrose, raffinose, stachyose, verbascose, monosaccharides, and combinations thereof. Minerals include but are not limited to calcium citrate.

Step 6 (See FIG. 4B) the protein washing and purification step can start with soy whey protein, BBI, KTI, storage proteins, other proteins or purified pre-treated whey from stream 5a. It includes a diafiltration step. Process variables and alternatives in this step include but are not limited to, reslurrying, crossflow membrane filtration, ultrafiltration, water diafiltration, buffer diafiltration, and combinations thereof. Crossflow membrane filtration includes but is not limited to: spiral-wound, plate and frame, hollow fiber, ceramic, dynamic or rotating disk, nanofiber, and combinations thereof. Processing aids that can be used in the protein washing and purification step include but are not limited to, water, steam, and combinations thereof. The pH of step 6 can be between about 2.0 and about 12.0, preferably about 7.0. The temperature can be between about 5° C. and about 90° C., preferably about 75° C. Products from stream 6a (retentate) include but are not limited to, soy whey protein, BBI, KTI, storage proteins, other proteins, and combinations thereof. Other proteins include but are not limited to lunasin, lectins, dehydrins, lipoxygenase, and combinations thereof. Products from stream 6b (permeate) include but are not limited to, peptides, soy oligosaccharides, water, minerals, and combinations thereof. Soy oligosaccharides include but are not limited to sucrose, raffinose, stachyose, verbascose, monosaccharides, and combinations thereof. Minerals include but are not limited to calcium citrate.

Step 16 (See FIG. 4B) a heat treatment and flash cooling step can start with soy whey protein, BBI, KTI and, other proteins from stream 6a. Other proteins include but are not limited to lunasin, lectins, dehydrins, lipoxygenase, and combinations thereof. It includes an ultra high temperature step. Process variables and alternatives in this step include but are not limited to, heat sterilization, evaporation, and combinations thereof. Processing aids that can be used in this heat treatment and flash cooling step include but are not limited to, water, steam, and combinations thereof. The temperature can be between about 129° C. and about 160° C., preferably about 152° C. Temperature hold time can be between about 8 seconds and about 15 seconds, preferably about 9 seconds. Products from stream 16 include but are not limited to, soy whey protein.

Finally, Step 17 (See FIG. 4B) a drying step can start with soy whey protein, BBI, KTI and, other proteins from stream 16. It includes a drying step. The liquid feed temperature can be between about 50° C. and about 95° C., preferably about 82° C. The inlet temperature can be between about 175° C. and about 370° C., preferably about 290° C. The exhaust temperature can be between about 65° C. and about 98° C., preferably about 88° C. Products from stream 17a (retentate) include but are not limited to, water. Products from stream 17b (permeate) include but are not limited to, soy whey protein which includes, BBI, KTI and, other proteins. Other proteins include but are not limited to lunasin, lectins, dehydrins, lipoxygenase, and combinations thereof.

Embodiment 16 starts with Step 0 (See FIG. 4A) the whey protein pretreatment can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 can be between about 3.0 and about 6.0, preferably 4.5. The temperature can be between about 70° C. and about 95° C., preferably about 85° C. Temperature hold times can vary between about 0 minutes to about 20 minutes, preferably about 10 minutes. Products from the whey protein pretreatment include but are not limited to soluble components in the aqueous phase of the whey stream (pre-treated soy whey) (molecular weight of equal to or less than about 50 kDa in stream 0a (retentate) and insoluble large molecular weight proteins (between about 300 kDa and between about 50 kDa in stream 0b (permeate), such as pre-treated soy whey, storage proteins, and combinations thereof.

Step 3 (See FIG. 4A) the mineral precipitation step can start with pretreated soy whey from stream 0a. It includes a precipitation step by pH and/or temperature change. Process variables and alternatives in this step include but are not limited to, an agitated or recirculating reaction tank. Processing aids that can be used in the mineral precipitation step include but are not limited to, acids, bases, calcium hydroxide, sodium hydroxide, hydrochloric acid, sodium chloride, phytase, and combinations thereof. The pH of step 3 can be between about 2.0 and about 12.0, preferably about 8.0. The temperature can be between about 5° C. and about 90° C., preferably about 50° C. The pH hold times can vary between about 0 minutes to about 60 minutes, preferably about 10 minutes. The product of stream 3 is a suspension of purified pre-treated soy whey and precipitated minerals.

Step 4 (See FIG. 4A) the mineral removal step can start with the suspension of purified pre-treated whey and precipitated minerals from stream 3. It includes a centrifugation step. Process variables and alternatives in this step include but are not limited to, centrifugation, filtration, dead-end filtration, crossflow membrane filtration and combinations thereof. Crossflow membrane filtration includes but is not limited to: spiral-wound, plate and frame, hollow fiber, ceramic, dynamic or rotating disk, nanofiber, and combinations thereof. Products from the mineral removal step include but are not limited to a de-mineralized pre-treated whey in stream 4a (retentate) and insoluble minerals with some protein mineral complexes in stream 4b (permeate). p Step 2 (See FIG. 4A) a water and mineral removal can start with the purified pre-treated soy whey from stream 4a. It includes a nanofiltration step for water removal and partial mineral removal. Process variables and alternatives in this step include but are not limited to, crossflow membrane filtration, reverse osmosis, evaporation, nanofiltration, and combinations thereof. Crossflow membrane filtration includes but is not limited to: spiral-wound, plate and frame, hollow fiber, ceramic, dynamic or rotating disk, nanofiber, and combinations thereof. The pH of step 2 can be between about 2.0 and about 12.0, preferably about 5.3. The temperature can be between about 5° C. and about 90° C., preferably about 50° C. Products from this water removal step include but are not limited to purified pre-treated soy whey in stream 2a (retentate) and water, some minerals, monovalent cations and combinations thereof in stream 2b (permeate).

Step 5 (See FIG. 4B) the protein separation and concentration step can start with the whey from stream 2a. It includes an ultrafiltration step. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, preferably about 8.0. The temperature can be between about 5° C. and about 90° C., preferably about 75° C. Products from stream 5a (retentate) include but are not limited to, soy whey protein, BBI, KTI, storage proteins, other proteins and combinations thereof. Other proteins include but are not limited to lunasin, lectins, dehydrins, lipoxygenase, and combinations thereof. Products from stream 5b (permeate) include but are not limited to, peptides, soy oligosaccharides, minerals and combinations thereof. Soy oligosaccharides include but are not limited to sucrose, raffinose, stachyose, verbascose, monosaccharides, and combinations thereof. Minerals include but are not limited to calcium citrate.

Step 6 (See FIG. 4B) the protein washing and purification step can start with soy whey protein, BBI, KTI, storage proteins, other proteins or purified pre-treated whey from stream 5a. It includes a diafiltration step. Process variables and alternatives in this step include but are not limited to, reslurrying, crossflow membrane filtration, ultrafiltration, water diafiltration, buffer diafiltration, and combinations thereof. Crossflow membrane filtration includes but is not limited to: spiral-wound, plate and frame, hollow fiber, ceramic, dynamic or rotating disk, nanofiber, and combinations thereof. Processing aids that can be used in the protein washing and purification step include but are not limited to, water, steam, and combinations thereof. The pH of step 6 can be between about 2.0 and about 12.0, preferably about 7.0. The temperature can be between about 5° C. and about 90° C., preferably about 75° C. Products from stream 6a (retentate) include but are not limited to, soy whey protein, BBI, KTI, storage proteins, other proteins, and combinations thereof. Other proteins include but are not limited to lunasin, lectins, dehydrins, lipoxygenase, and combinations thereof. Products from stream 6b (permeate) include but are not limited to, peptides, soy oligosaccharides, water, minerals, and combinations thereof. Soy oligosaccharides include but are not limited to sucrose, raffinose, stachyose, verbascose, monosaccharides, and combinations thereof. Minerals include but are not limited to calcium citrate.

Step 15 (See FIG. 4B) a water removal step can start with soy whey protein, BBI, KTI and, other proteins from stream 6a. Other proteins include but are not limited to lunasin, lectins, dehydrins, lipoxygenase, and combinations thereof. It includes an evaporation step. Process variables and alternatives in this step include but are not limited to, evaporation, nanofiltration, RO, and combinations thereof. Products from stream 15a (retentate) include but are not limited to, water. Stream 15b permeate) products include but are not limited to soy whey protein, BBI, KTI and, other proteins. Other proteins include but are not limited to lunasin, lectins, dehydrins, lipoxygenase, and combinations thereof.

Step 16 (See FIG. 4B) a heat treatment and flash cooling step can start with soy whey protein, BBI, KTI and, other proteins from stream 15b. Other proteins include but are not limited to lunasin, lectins, dehydrins, lipoxygenase, and combinations thereof. It includes an ultra high temperature step. Process variables and alternatives in this step include but are not limited to, heat sterilization, evaporation, and combinations thereof. Processing aids that can be used in this heat treatment and flash cooling step include but are not limited to, water, steam, and combinations thereof. The temperature can be between about 129° C. and about 160° C., preferably about 152° C. Temperature hold time can be between about 8 seconds and about 15 seconds, preferably about 9 seconds. Products from stream 16 include but are not limited to, soy whey protein.

Finally, Step 17 (See FIG. 4B) a drying step can start with soy whey protein , BBI, KTI and, other proteins from stream 16. It includes a drying step. The liquid feed temperature can be between about 50° C. and about 95° C., preferably about 82° C. The inlet temperature can be between about 175° C. and about 370° C., preferably about 290° C. The exhaust temperature can be between about 65° C. and about 98° C., preferably about 88° C. Products from stream 17a (retentate) include but are not limited to, water. Products from stream 17b (permeate) include but are not limited to, soy whey protein which includes, BBI, KTI and, other proteins. Other proteins include but are not limited to lunasin, lectins, dehydrins, lipoxygenase, and combinations thereof.

Embodiment 17 starts with Step 0 (See FIG. 4A) the whey protein pretreatment can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 can be between about 3.0 and about 6.0, preferably 4.5. The temperature can be between about 70° C. and about 95° C., preferably about 85° C. Temperature hold times can vary between about 0 minutes to about 20 minutes, preferably about 10 minutes. Products from the whey protein pretreatment include but are not limited to soluble components in the aqueous phase of the whey stream (pre-treated soy whey) (molecular weight of equal to or less than about 50 kDa in stream 0a (retentate) and insoluble large molecular weight proteins (between about 300 kDa and between about 50 kDa in stream 0b (permeate), such as pre-treated soy whey, storage proteins, and combinations thereof.

Step 1 (See FIG. 4A) Microbiology reduction can start with the product of the whey protein pretreatment step, including but not limited to pre-treated soy whey. This step involves microfiltration of the pre-treated soy whey. Process variables and alternatives in this step include but are not limited to, centrifugation, dead-end filtration, heat sterilization, ultraviolet sterilization, microfiltration, crossflow membrane filtration, and combinations thereof. Crossflow membrane filtration includes but is not limited to: spiral-wound, plate and frame, hollow fiber, ceramic, dynamic or rotating disk, nanofiber, and combinations thereof. The pH of step 1 can be between about 2.0 and about 12.0, preferably about 5.3. The temperature can be between about 5° C. and about 90° C., preferably about 50° C. Products from step 1 include but are not limited to storage proteins, microorganisms, silicon, and combinations thereof in stream 1a (retentate) and purified pre-treated soy whey in stream 1b (permeate).

Step 3 (See FIG. 4A) the mineral precipitation step can start with pretreated soy whey from stream 1b. It includes a precipitation step by pH and/or temperature change. Process variables and alternatives in this step include but are not limited to, an agitated or recirculating reaction tank. Processing aids that can be used in the mineral precipitation step include but are not limited to, acids, bases, calcium hydroxide, sodium hydroxide, hydrochloric acid, sodium chloride, phytase, and combinations thereof. The pH of step 3 can be between about 2.0 and about 12.0, preferably about 8.0. The temperature can be between about 5° C. and about 90° C., preferably about 50° C. The pH hold times can vary between about 0 minutes to about 60 minutes, preferably about 10 minutes. The product of stream 3 is a suspension of purified pre-treated soy whey and precipitated minerals.

Step 4 (See FIG. 4A) the mineral removal step can start with the suspension of purified pre-treated whey and precipitated minerals from stream 3. It includes a centrifugation step. Process variables and alternatives in this step include but are not limited to, centrifugation, filtration, dead-end filtration, crossflow membrane filtration and combinations thereof. Crossflow membrane filtration includes but is not limited to: spiral-wound, plate and frame, hollow fiber, ceramic, dynamic or rotating disk, nanofiber, and combinations thereof. Products from the mineral removal step include but are not limited to a de-mineralized pre-treated whey in stream 4a (retentate) and insoluble minerals with some protein mineral complexes in stream 4b (permeate).

Step 2 (See FIG. 4A)—A water and mineral removal can start with the purified pre-treated soy whey from stream 4a. It includes a nanofiltration step for water removal and partial mineral removal. Process variables and alternatives in this step include but are not limited to, crossflow membrane filtration, reverse osmosis, evaporation, nanofiltration, and combinations thereof. Crossflow membrane filtration includes but is not limited to: spiral-wound, plate and frame, hollow fiber, ceramic, dynamic or rotating disk, nanofiber, and combinations thereof. The pH of step 2 can be between about 2.0 and about 12.0, preferably about 5.3. The temperature can be between about 5° C. and about 90° C., preferably about 50° C. Products from this water removal step include but are not limited to purified pre-treated soy whey in stream 2a (retentate) and water, some minerals, monovalent cations and combinations thereof in stream 2b (permeate).

Step 5 (See FIG. 4B) the protein separation and concentration step can start with the whey from stream 2a. It includes an ultrafiltration step. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, preferably about 8.0. The temperature can be between about 5° C. and about 90° C., preferably about 75° C. Products from stream 5a (retentate) include but are not limited to, soy whey protein, BBI, KTI, storage proteins, other proteins and combinations thereof. Other proteins include but are not limited to lunasin, lectins, dehydrins, lipoxygenase, and combinations thereof. Products from stream 5b (permeate) include but are not limited to, peptides, soy oligosaccharides, minerals and combinations thereof. Soy oligosaccharides include but are not limited to sucrose, raffinose, stachyose, verbascose, monosaccharides, and combinations thereof. Minerals include but are not limited to calcium citrate.

Step 6 (See FIG. 4B) the protein washing and purification step can start with soy whey protein, BBI, KTI, storage proteins, other proteins or purified pre-treated whey from stream 5a. It includes a diafiltration step. Process variables and alternatives in this step include but are not limited to, reslurrying, crossflow membrane filtration, ultrafiltration, water diafiltration, buffer diafiltration, and combinations thereof. Crossflow membrane filtration includes but is not limited to: spiral-wound, plate and frame, hollow fiber, ceramic, dynamic or rotating disk, nanofiber, and combinations thereof. Processing aids that can be used in the protein washing and purification step include but are not limited to, water, steam, and combinations thereof. The pH of step 6 can be between about 2.0 and about 12.0, preferably about 7.0. The temperature can be between about 5° C. and about 90° C., preferably about 75° C. Products from stream 6a (retentate) include but are not limited to, soy whey protein, BBI, KTI, storage proteins, other proteins, and combinations thereof. Other proteins include but are not limited to lunasin, lectins, dehydrins, lipoxygenase, and combinations thereof. Products from stream 6b (permeate) include but are not limited to, peptides, soy oligosaccharides, water, minerals, and combinations thereof. Soy oligosaccharides include but are not limited to sucrose, raffinose, stachyose, verbascose, monosaccharides, and combinations thereof. Minerals include but are not limited to calcium citrate.

Step 15 (See FIG. 4B) a water removal step can start with soy whey protein, BBI, KTI and, other proteins from stream 6a. Other proteins include but are not limited to lunasin, lectins, dehydrins, lipoxygenase, and combinations thereof. It includes an evaporation step. Process variables and alternatives in this step include but are not limited to, evaporation, nanofiltration, reverse osmosis, and combinations thereof. Products from stream 15a (retentate) include but are not limited to, water. Stream 15b (permeate) products include but are not limited to soy whey protein, BBI, KTI and, other proteins. Other proteins include but are not limited to lunasin, lectins, dehydrins, lipoxygenase, and combinations thereof.

Step 16 (See FIG. 4B) a heat treatment and flash cooling step can start with soy whey protein, BBI, KTI and, other proteins from stream 15b. Other proteins include but are not limited to lunasin, lectins, dehydrins, lipoxygenase, and combinations thereof. It includes an ultra high temperature step. Process variables and alternatives in this step include but are not limited to, heat sterilization, evaporation, and combinations thereof. Processing aids that can be used in this heat treatment and flash cooling step include but are not limited to, water, steam, and combinations thereof. The temperature can be between about 129° C. and about 160° C., preferably about 152° C. Temperature hold time can be between about 8 seconds and about 15 seconds, preferably about 9 seconds. Products from stream 16 include but are not limited to, soy whey protein.

Finally, Step 17 (See FIG. 4B) a drying step can start with soy whey protein, BBI, KTI and, other proteins from stream 16. It includes a drying step. The liquid feed temperature can be between about 50° C. and about 95° C., preferably about 82° C. The inlet temperature can be between about 175° C. and about 370° C., preferably about 290° C. The exhaust temperature can be between about 65° C. and about 98° C., preferably about 88° C. Products from stream 17a (retentate) include but are not limited to, water. Products from stream 17b (permeate) include but are not limited to, soy whey protein which includes, BBI, KTI and, other proteins. Other proteins include but are not limited to lunasin, lectins, dehydrins, lipoxygenase, and combinations thereof.

Embodiment 18 starts with Step 0(See FIG. 4A) the whey protein pretreatment can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 can be between about 3.0 and about 6.0, preferably 4.5. The temperature can be between about 70° C. and about 95° C., preferably about 85° C. Temperature hold times can vary between about 0 minutes to about 20 minutes, preferably about 10 minutes. Products from the whey protein pretreatment include but are not limited to soluble components in the aqueous phase of the whey stream (pre-treated soy whey) (molecular weight of equal to or less than about 50 kDa in stream 0a (retentate) and insoluble large molecular weight proteins (between about 300 kDa and between about 50 kDa in stream 0b (permeate), such as pre-treated soy whey, storage proteins, and combinations thereof.

Step 1 (See FIG. 4A) Microbiology reduction can start with the product of the whey protein pretreatment step, including but not limited to pre-treated soy whey. This step involves microfiltration of the pre-treated soy whey. Process variables and alternatives in this step include but are not limited to, centrifugation, dead-end filtration, heat sterilization, ultraviolet sterilization, microfiltration, crossflow membrane filtration, and combinations thereof. Crossflow membrane filtration includes but is not limited to: spiral-wound, plate and frame, hollow fiber, ceramic, dynamic or rotating disk, nanofiber, and combinations thereof. The pH of step 1 can be between about 2.0 and about 12.0, preferably about 5.3. The temperature can be between about 5° C. and about 90° C., preferably about 50° C. Products from step 1 include but are not limited to storage proteins, microorganisms, silicon, and combinations thereof in stream 1a (retentate) and purified pre-treated soy whey in stream 1b (permeate).

Step 2 (See FIG. 4A) a water and mineral removal can start with the purified pre-treated soy whey from stream 1b. It includes a nanofiltration step for water removal and partial mineral removal. Process variables and alternatives in this step include but are not limited to, crossflow membrane filtration, reverse osmosis, evaporation, nanofiltration, and combinations thereof. Crossflow membrane filtration includes but is not limited to: spiral-wound, plate and frame, hollow fiber, ceramic, dynamic or rotating disk, nanofiber, and combinations thereof. The pH of step 2 can be between about 2.0 and about 12.0, preferably about 5.3. The temperature can be between about 5° C. and about 90° C., preferably about 50° C. Products from this water removal step include but are not limited to purified pre-treated soy whey in stream 2a (retentate) and water, some minerals, monovalent cations and combinations thereof in stream 2b (permeate).

Step 3 (See FIG. 4A) the mineral precipitation step can start with purified pre-treated soy whey from stream 2a. It includes a precipitation step by pH and/or temperature change. Process variables and alternatives in this step include but are not limited to, an agitated or recirculating reaction tank. Processing aids that can be used in the mineral precipitation step include but are not limited to, acids, bases, calcium hydroxide, sodium hydroxide, hydrochloric acid, sodium chloride, phytase, and combinations thereof. The pH of step 3 can be between about 2.0 and about 12.0, preferably about 8.0. The temperature can be between about 5° C. and about 90° C., preferably about 50° C. The pH hold times can vary between about 0 minutes to about 60 minutes, preferably about 10 minutes. The product of stream 3 is a suspension of purified pre-treated soy whey and precipitated minerals.

Step 4 (See FIG. 4A)—the mineral removal step can start with the suspension of purified pre-treated whey and precipitated minerals from stream 3. It includes a centrifugation step. Process variables and alternatives in this step include but are not limited to, centrifugation, filtration, dead-end filtration, crossflow membrane filtration and combinations thereof. Crossflow membrane filtration includes but is not limited to: spiral-wound, plate and frame, hollow fiber, ceramic, dynamic or rotating disk, nanofiber, and combinations thereof. Products from the mineral removal step include but are not limited to a de-mineralized pre-treated whey in stream 4a (retentate) and insoluble minerals with some protein mineral complexes in stream 4b (permeate).

Step 5 (See FIG. 4B) the protein separation and concentration step can start with purified pre-treated whey from stream 4a. It includes an ultrafiltration step. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, preferably about 8.0. The temperature can be between about 5° C. and about 90° C., preferably about 75° C. Products from stream 5a (retentate) include but are not limited to, soy whey protein, BBI, KTI, storage proteins, other proteins and combinations thereof. Other proteins include but are not limited to lunasin, lectins, dehydrins, lipoxygenase, and combinations thereof. Products from stream 5b (permeate) include but are not limited to, peptides, soy oligosaccharides, minerals and combinations thereof. Soy oligosaccharides include but are not limited to sucrose, raffinose, stachyose, verbascose, monosaccharides, and combinations thereof. Minerals include but are not limited to calcium citrate.

Step 6 (See FIG. 4B) the protein washing and purification step can start with soy whey protein, BBI, KTI, storage proteins, other proteins or purified pre-treated whey from stream 5a. It includes a diafiltration step. Process variables and alternatives in this step include but are not limited to, reslurrying, crossflow membrane filtration, ultrafiltration, water diafiltration, buffer diafiltration, and combinations thereof. Crossflow membrane filtration includes but is not limited to: spiral-wound, plate and frame, hollow fiber, ceramic, dynamic or rotating disk, nanofiber, and combinations thereof. Processing aids that can be used in the protein washing and purification step include but are not limited to, water, steam, and combinations thereof. The pH of step 6 can be between about 2.0 and about 12.0, preferably about 7.0. The temperature can be between about 5° C. and about 90° C., preferably about 75° C. Products from stream 6a (retentate) include but are not limited to, soy whey protein, BBI, KTI, storage proteins, other proteins, and combinations thereof. Other proteins include but are not limited to lunasin, lectins, dehydrins, lipoxygenase, and combinations thereof. Products from stream 6b (permeate) include but are not limited to, peptides, soy oligosaccharides, water, minerals, and combinations thereof. Soy oligosaccharides include but are not limited to sucrose, raffinose, stachyose, verbascose, monosaccharides, and combinations thereof. Minerals include but are not limited to calcium citrate.

Step 15 (See FIG. 4B) a water removal step can start with soy whey protein, BBI, KTI and, other proteins from stream 6a. Other proteins include but are not limited to lunasin, lectins, dehydrins, lipoxygenase, and combinations thereof. It includes an evaporation step. Process variables and alternatives in this step include but are not limited to, evaporation, nanofiltration, reverse osmosis, and combinations thereof. Products from stream 15a (retentate) include but are not limited to, water. Stream 15b (permeate) products include but are not limited to soy whey protein, BBI, KTI and, other proteins. Other proteins include but are not limited to lunasin, lectins, dehydrins, lipoxygenase, and combinations thereof.

Step 16 (See FIG. 4B) a heat treatment and flash cooling step can start with soy whey protein, BBI, KTI and, other proteins from stream 15b. Other proteins include but are not limited to lunasin, lectins, dehydrins, lipoxygenase, and combinations thereof. It includes an ultra high temperature step. Process variables and alternatives in this step include but are not limited to, heat sterilization, evaporation, and combinations thereof. Processing aids that can be used in this heat treatment and flash cooling step include but are not limited to, water, steam, and combinations thereof. The temperature can be between about 129° C. and about 160° C., preferably about 152° C. Temperature hold time can be between about 8 seconds and about 15 seconds, preferably about 9 seconds. Products from stream 16 include but are not limited to, soy whey protein.

Finally, Step 17 (See FIG. 4B) a drying step can start with soy whey protein, BBI, KTI and, other proteins from stream 16. It includes a drying step. The liquid feed temperature can be between about 50° C. and about 95° C., preferably about 82° C. The inlet temperature can be between about 175° C. and about 370° C., preferably about 290° C. The exhaust temperature can be between about 65° C. and about 98° C., preferably about 88° C. Products from stream 17a (retentate) include but are not limited to, water. Products from stream 17b (permeate) include but are not limited to, soy whey protein which includes, BBI, KTI and, other proteins. Other proteins include but are not limited to lunasin, lectins, dehydrins, lipoxygenase, and combinations thereof.

### V. Personal Care Products and Industrial Products Comprising a Foaming Agent

The present disclosure further relates to products that contain a foaming agent comprising an amount of soy whey protein having a soluble solids index (SSI) of at least about 80% across a pH range of from 2 to 10 and a temperature of 25° C. The foaming agent disclosed herein is suitable for use in a variety of products, but is especially suitable for use in products requiring aeration, such as, for example, personal care products, industrial products, household products, flame retardant agent, foam insulation, foam mattress, foam rubber, foam packaging, and the like. One of skill in the art will appreciate that the amount of foaming agent used can and will vary depending upon the type of product.

In one embodiment, the product comprising the foaming agent may be a personal care product, such as hand soap, face soap, body soap, shaving foam, toothpaste, shampoo, hair color products, and hair styling foam.

In another embodiment, the product comprising the foaming agent may be an industrial or household product, such as a cleaner, soap, detergent, foam mattress, foam cushion, and foam pillow.

In another embodiment, the product comprising the foaming agent may be flame retardant foam.

In another embodiment, the product comprising the foaming agent may be a packaging material such as packing foam.

In another embodiment, the product comprising the foaming agent may be foam insulation for residential or commercial use.

Typically, the amount of foaming agent present in the product can and will vary depending on the desired product and the amount of foam needed to make the product. By way of example, a personal care product may contain between about 0.02% and about 10% (by weight) of a foaming agent. Specifically, a personal care product may contain about 10%, 9%, 8%, 7%, 6%, 5%, 4%, 3%, 2.5%, 2%, 1.5%, 1%, 0.50%, 0.25%, 0.1%, 0.05%, or 0.02% (by weight) of a foaming agent. In one embodiment, the amount of foaming agent present in the product may range from about 0.02% to about 3% by weight. Additionally, the amount of foaming agent present in the product may comprise between about 0.02% to about 2% by weight.

The foaming agent may be added at the initial hydration step or to the pre-mix or at a subsequent processing step in the preparation of the final product. In one embodiment, the foaming agent is added in water as part of the initial hydration of the protein followed by the addition of other ingredients. In an alternative embodiment, the foaming agent is added to the dry ingredients in a dry form as part of the dry blend pre-mix before adding to the liquid ingredients.

a. Additional Ingredients

Additional ingredients may be combined with the foaming agent to form a desired product. One of skill in the art will appreciate that the specific additional ingredients will vary depending on the type of industrial product to be made. Examples of additional ingredients that may be combined with the foaming agent containing an amount of soy whey protein, include thickening agents, water, moisturizing agents, abrasives, stain removing agents, fragrances, pigments, viscosity control agents, preservatives, humectants, other additives, and combinations thereof.

1. Additional Foaming Agent

The industrial product may optionally contain at least one additional foaming agent to inhibit the separation of the industrial product into air and water phases. The additional foaming agent may be a surfactant. Because the soy whey proteins of the present invention have been found to further exhibit stabilizing properties in addition to foaming properties, additional foaming agents may not be needed. However, non-limiting examples of suitable foaming agents in the art that may be used in addition to soy whey protein include mono- & diglycerides of fatty acids, esters of monoglycerides of fatty acids, mono- & diglycerides, propylene glycol monoesters, lecithin, hydroxylated lecithin, dioctyl sodium sulphosuccinate, SSL, CSL, Polysorbate 20, Polysorbate 40, Polysorbate 60, Polysorbate 80, sorbitan tristearate, stearyl citrate, PGPR, lactylates, SLES, SDS, ALS, cocamide diethanolamine, triethanolamine, sodium lauroyl sarcosinate, and combinations thereof.

By way of example, if added, the additional foaming agent(s) may be present in a personal care product or industrial product at a level from about 0.1% to about 10% and preferably from about 1% to about 5% by weight of the product. As will be appreciated by one of skill in the art, the amount of additional foaming agent, if any, added to the product can and will depend upon the type of product desired (e.g., personal care product, industrial product, etc.).

2. Thickening Agent

The industrial product may optionally include a thickening agent depending on the desired final product to be produced. Suitable thickening agents may include carrageenan, cellulose gum, cellulose gel, starch, low DE maltodextrin, gum arabic, xanthan gum, and any other thickening agent known and used in the industry. The thickening agent may be present in the industrial product at levels from about 0.01% to about 10%, preferably from about 0.05% to about 5%, and more preferably from about 0.1% to about 2% by weight of the product. As will be appreciated by a skilled artisan, the amount of thickening agent, if any, added to the product can and will depend upon the type of product desired.

3. Fragrance

The product may optionally include a variety of fragrances to naturally enhance the aroma of the final product. As will be appreciated by a skilled artisan, the selection of fragrances added to a product can and will depend upon the type of product desired (e.g., personal care product, industrial product, etc.).

Perfume oils which may be added can be mixtures of natural and synthetic fragrances. Natural fragrances are extracts from flowers (lily, lavender, rose, jasmine, neroli, ylang-ylang), stems and leaves (geranium, patchouli, petitgrain), fruits (anise, coriander, caraway, juniper), fruit peels (bergamot, lemons, oranges), roots (mace, angelica, celery, cardamom, costus, iris, thyme), needles and branches (spruce, fir, pine, dwarf-pine), resins and balsams (galbanum, elemi, benzoin, myrrh, olibanum, opoponax). Animal raw materials are also suitable, such as, for example, civet and castoreum. Typical synthetic fragrance compounds are products of the ester, ether, aldehyde, ketone, alcohol and hydrocarbon type. Fragrance compounds of the ester type are, for example, benzyl acetate, phenoxyethyl isobutyrate, p-tert-butyl cyclohexylacetate, linalyl acetate, dimethylbenzylcarbinyl acetate, phenylethyl acetate, linalyl benzoate, benzyl formate, ethylmethyl phenylglycinate, allyl cyclohexylpropionate, styrallyl propionate and benzyl salicylate. The ethers include, for example, benzyl ethyl ether, the aldehydes include, for example, the linear alkanals having 8 to 18 carbon atoms, citral, citronellal, citronellyloxyacetaldehyde, cyclamenaldehyde, hydroxycitronellal, lilial and bourgeonal, the ketones include, for example, the ionones, .alpha.-isomethylionone and methyl cedryl ketone, the alcohols include anethol, citronellol, eugenol, isoeugenol, geraniol, linalool, phenylethyl alcohol and terpineol, and the hydrocarbons include primarily the terpenes and balsams. However, preference is given to using mixtures of different fragrances which together produce a pleasing scent note. Essential oils of low volatility, which are mostly used as aroma components, are also suitable as perfume oils, for example sage oil, camomile oil, oil of cloves, melissa oil, mint oil, cinnamon leaf oil, linden blossom oil, juniper berry oil, vetiver oil, olibanum oil, galbanum oil, labdanum oil and lavandin oil.

4. Pigments

In an additional embodiment, the product may further comprise a pigment. The pigments are colorants which are virtually insoluble in the application medium and may be inorganic or organic. Inorganic-organic mixed pigments are also possible. Preference is given to inorganic pigments. The advantage of the inorganic pigments is their excellent fastness to light, weather and temperature. The inorganic pigments may be of natural origin, for example prepared from chalk, ocker, umbra, green earth, burnt sienna or graphite. The pigments may be white pigments, such as, for example, titanium dioxide or zinc oxide, black pigments, such as, for example, iron oxide black, colored pigments, such as, for example ultramarine or iron oxide red, luster pigments, metal effect pigments, pearlescent pigments, and fluorescent and phosphorescent pigments, where preferably at least one pigment is a colored, nonwhite pigment.

Metal oxides, hydroxides and oxide hydrates, mixed phase pigments sulfur-containing silicates, metal sulfides, complex metal cyanides, metal sulfates, chromates and molybdates, and also the metals themselves (bronze pigments) are suitable. Of particular suitability are titanium dioxide (CI 77891), black iron oxide (CI 77499), yellow iron oxide (CI 77492), red and brown iron oxide (CI 77491), manganese violet (CI 77742), ultramarine (sodium aluminum sulfosilicates, CI 77007, pigment blue 29), chromium oxide hydrate (C177289), iron blue (ferric ferrocyanide, C17751 0), carmine (cochineal).

Particular preference is given to pearlescent and colored pigments based on mica which are coated with a metal oxide or a metal oxychloride such as titanium dioxide or bismuth oxychloride, and, if appropriate, further color-imparting substances, such as iron oxides, iron blue, ultramarine, carmine etc., and where the color can be determined by varying the layer thickness. Pigments of this type are sold, for example, under the trade names Rona®, Colorona®, Dichrona® and Timiron® (Merck).

Organic pigments are, for example, the natural pigments sepia, gamboge, charcoal, Cassel brown, indigo, chlorophyll, phylloxanthins, such as astaxanthin and cryptoxanthin, and other plant pigments. Synthetic organic pigments are, for example, azo pigments, anthraquinoids, indigoids, dioxazine, quinacridone, phthalocyanine, isoindolinone, perylene and perinone, metal complex, alkali blue and diketopyrrolopyrrole pigments.

These pigments may be combined or mixed as is common to those skilled in the art to produce a final pigment.

### VI. Method of Making Personal Care and Industrial Products

As referenced herein, the personal care and industrial products comprising a foaming agent containing an amount of soy whey protein may undergo typical processing known in the industry to produce the desired final product. Generally speaking, any method of processing known in the industry can be used to produce the desired personal care products and industrial products.

### DEFINITIONS

To facilitate understanding of the invention, several terms are defined below.

The term “acid soluble” as used herein refers to a substance having a solubility of at least about 80% with a concentration of 10 grams per liter (g/L) in an aqueous medium having a pH of from about 2 to about 7.

The terms “soy protein isolate” or “isolated soy protein,” as used herein, refer to a soy material having a protein content of at least about 90% soy protein on a moisture free basis.

The term “soluble solids index” or “SSI” as used herein refers to the solubility of a soy protein material in an aqueous solution as measured according to the following formula: SSI (%)=(Soluble Solids/Total Solids)×100. Soluble Solids and Total Solids are determined as provided in Example 15.

The term “other proteins” as used herein referred to throughout the application are defined as including but not limited to: lunasin, lectins, dehydrins, lipoxygenase, and combinations thereof.

The term “soy whey protein” as used herein is defined as including protein soluble at those pHs where soy storage proteins are typically insoluble, including but not limited to BBI, KTI, lunasin, lipoxygenase, dehydrins, lectins, and combinations thereof. Soy whey protein may further include storage proteins.

The term “processing stream” as used herein refers to the secondary or incidental product derived from the process of refining a whole legume or oilseed, including an aqueous or solvent stream, which includes, for example, an aqueous soy extract stream, an aqueous soymilk extract stream, an aqueous soy whey stream, an aqueous soy molasses stream, an aqueous soy protein concentrate soy molasses stream, an aqueous soy permeate stream, and an aqueous tofu whey stream, and additionally includes soy whey protein, for example, in both liquid and dry powder form, that can be recovered as an intermediate product in accordance with the methods disclosed herein.

The term “personal care products” and “industrial products” as used herein broadly refers to a mixture of a combination of safe and suitable ingredients including, but not limited to, a foaming agent containing an amount of soy whey protein. Other additives, such as additional foaming agents, thickening agents, preservatives, pigments, and fragrances, may also be included.

The term “proteins other than soy whey protein” is defined as any animal or vegetable protein other than soy whey protein.

When introducing elements of the present invention or the preferred embodiments(s) thereof, the articles “a,” “an,” “the” and “said” are intended to mean that there are one or more of the elements. The terms “comprising,” “including” and “having” are intended to be inclusive and mean that there may be additional elements other than the listed elements.

The term “invention” or “present invention” as used herein is a non-limiting term and is not intended to refer to any single embodiment of the particular invention but encompasses all possible embodiments as described in the specification and the claims.

As used herein, the term “about” modifying the quantity of an ingredient of the invention employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities.

As various changes could be made in the above compounds, products and methods without departing from the scope of the invention, it is intended that all matter contained in the above description and in the examples given below, shall be interpreted as illustrative and not in a limiting sense.

### EXAMPLES

### Example 1

### Recovery and Fractionation of Soy Whey Protein From Aqueous Soy Whey Using Novel Membrane Process

145 liters of aqueous raw soy whey (not pre-treated) with a total solids content of 3.7% and dry basis protein content of 19.8% was microfiltered using two different membranes in an OPTISEP® 7000 module, manufactured by SmartFlow Technologies. The first membrane, BTS-25, was a polysulfone construction with 0.5 um pore size manufactured by Pall. Aqueous soy whey was concentrated to a 1.6× factor, at an average flux of 30 liters/meter2/hr (LMH). The concentrated aqueous soy whey was then passed through a modified polysulfone microfiltration membrane, MPS 0.45, manufactured by Pall. The aqueous soy whey was concentrated from 1.6× to 11× at an average flux of 28 LMH.

Permeate from the microfiltration process, 132 liters total, was then introduced into an OPTISEP® 7000 module with ultrafiltration membranes, RC100, which are 100 kDa regenerated cellulose membranes manufactured by Microdyn-Nadir. The microfiltered aqueous soy whey was concentrated to about 20× using a 20 L tank setup at an average flux of 30 LMH before being transferred to a 5 L tank setup in order to minimize the hold-up volume of the system. In the smaller tank, the aqueous soy whey was concentrated from 20× to 66× at an average flux rate of 9 LMH, reaching a final retentate volume of 2 liters. The final retentate was 24.0% total solids, and 83.0% dry basis protein content.

128 liters of sugar and mineral enriched RC100 permeate was then introduced into an OPTISEP® 7000 module with polysulfone thin film nanofiltration membranes with a 35% NaCl rejection rate, NF20, manufactured by Sepro. The feed was concentrated 18× at an average flux rate of 4.7 LMH. The retentate from this process step, 9 liters, was enriched in the various sugar species. The permeate stream from the NF20 separation process, 121 liters, contained the minerals and water.

The permeate of the NF20 process was then introduced into an OPTISEP® 3000 module with thin film reverse osmosis membranes with a 98.2% NaCl rejection rate, SG, manufactured by GE. The feed was concentrated 12× at an average flux rate of 8 LMH. The permeate of the SG membrane, 9.2 liters, consisted primarily of water, suitable for re-use in a process with minimal further treatment. The retentate of the SG process, 0.8 liters, consisted predominantly of a concentrated mineral fraction.

### Example 2

### Recovery and Fractionation of Soy Whey Protein from Soy Molasses Using Novel Membrane Process

61.7 liters of soy molasses with a total solids content of 62.7% and dry basis protein content of 18.5% was diluted with 61.7 liters of water prior to microfiltration. The diluted soy molasses was then microfiltered using an OPTISEP® 7000 module, manufactured by SmartFlow Technologies. The diluted soy molasses passed through a modified polysulfone microfiltration membrane, MPS 0.45, manufactured by Pall. The diluted soy molasses was concentrated to a 1.3× factor, at an average flux of 6 liters/meter2/hr (LMH).

Permeate from the microfiltration process, 25 liters total, was then introduced into an OPTISEP® 7000 module with ultrafiltration membranes, RC100, which are 100 kDa regenerated cellulose membranes manufactured by Microdyn-Nadir. The microfiltered diluted soy molasses was diafiltered with 2 volumes of water prior to being concentrated to 7.6× at an average flux of 20 LMH, reaching a final retentate volume of 2 liters. The final retentate was 17.5% total solids, and 22.0% dry basis protein content.

72 liters of sugar and mineral enriched RC100 permeate was then introduced into an OPTISEP® 7000 module with polysulfone thin film nanofiltration membranes with a 35% NaCl rejection rate, NF20, manufactured by Sepro. The feed was concentrated 3× at an average flux rate of 4.0 LMH. The retentate from this process step, 23 liters, was enriched in the various sugar species. The permeate stream from the NF20 separation process, 48 liters, contained the minerals and water.

A portion of the permeate of the NF20 process, 10 liters, was then introduced into an OPTISEP® 3000 module with thin film reverse osmosis membranes with a 98.2% NaCl rejection rate, SG, manufactured by GE. The feed was concentrated 6.7× at an average flux rate of 7.9 LMH. The permeate of the SG membrane, 8.5 liters, consisted primarily of water, suitable for re-use in a process with minimal further treatment. The retentate of the SG process, 1.5 liters, consisted predominantly of a concentrated mineral fraction.

### Example 3

### Capture of Bulk Soy Whey Protein From Defatted Soy Flour Extract

Defatted soy flour (DSF) was extracted by adding a 15:1 ratio of water to DSF at a pH of 7.8 and stirring for 20 minutes prior to filtration. The extract was microfiltered using an OPTISEP® 800 module, manufactured by SmartFlow Technologies. The microfiltration membrane, MMM-0.8, was a polysulfone and polyvinylpropylene construction with 0.8 um pore size manufactured by Pall. Aqueous soy extract was concentrated to a 2.0× factor, at an average flux of 29 liters/meter2/hr (LMH). Permeate from the microfiltration process was then introduced into an OPTISEP® 800 module with ultrafiltration membranes, RC100, which are 100 kDa regenerated cellulose membranes manufactured by Microdyn-Nadir. The microfiltered aqueous soy extract was concentrated to about 6.3× at an average flux rate of 50 LMH. The final retentate measured 84.7% dry basis protein content.

### Example 4

### Capture of Bulk Soy Whey Protein Using Continuous Separation Technology CSEP (Simulated Moving Bed Chromatography)

CSEP experiments were performed by passing feed material (soy whey) through a column (ID 1.55 cm, length 9.5 cm, volume 18 mL) packed with SP GibcoCel resin. The column was connected to a positive displacement pump and samples of flow through and eluates were collected at the outlet of the column. Different experimental conditions were used to determine the effect of feed concentration, feed flow rate and elution flow rate on the binding capacity of the resin.

Feed Concentration

Soy whey was prepared from the defatted soy flake. Briefly, one part of defatted flake was mixed with 15 parts of water at 32° C. The pH of the solution was adjusted to 7.0 using 2 M NaOH and proteins were extracted into the aqueous phase by stirring the solution for 15 min. The protein extract was separated from the insoluble material by centrifugation at 3000×g for 10 min. The pH of the collected supernatant was adjusted to 4.5 using 1 M HCl and the solution was stirred for 15 min followed by heating to a temperature of 57° C. This treatment resulted in precipitation of the storage proteins while the whey proteins remained soluble. The precipitated proteins were separated from the whey by centrifugation at 3000×g for 10 min.

In some cases, the soy whey was concentrated using a Lab-Scale Amicon DC-10LA ultrafiltration unit and Amicon 3K membrane. Prior to ultrafiltration, pH of soy whey was adjusted to 5.5 with 2 M NaOH to avoid membrane fouling at acidic conditions. 10 L of whey was processed with the flux at ˜100 mL/min. Once the concentration factor of 5 in the retentate was reached, both retentate and permeate streams were collected. Soy whey concentrates 2.5×, 3×, and 4× were prepared by mixing a known amount of permeate and 5× whey concentrate. The pH of all soy concentrates was readjusted if necessary to 4.5.

Feed Flow Rate

During dynamic adsorption, as fluid flows through the resin bed, the proteins are adsorbed by the resin and reach equilibrium with the liquid phase. As the whey is loaded onto the column, the bound protein band extends down the column and reaches equilibrium with the liquid phase. When the resin is saturated with adsorbed proteins, the concentration of the proteins in the liquid phase exiting the column will be similar to the protein concentration in the feed. The curve describing the change in the flow through concentration compared to the feed concentration with the passage of fluid is the breakthrough curve. The concentration of protein in the solid phase increases as the breakthrough curve is developed, and the adsorption wave moves through the bed. As more fluid is passed through the bed, the flow through concentration increases asymptotically to the incoming fluid stream and at the same time a similar phenomena is achieved with the solid phase.

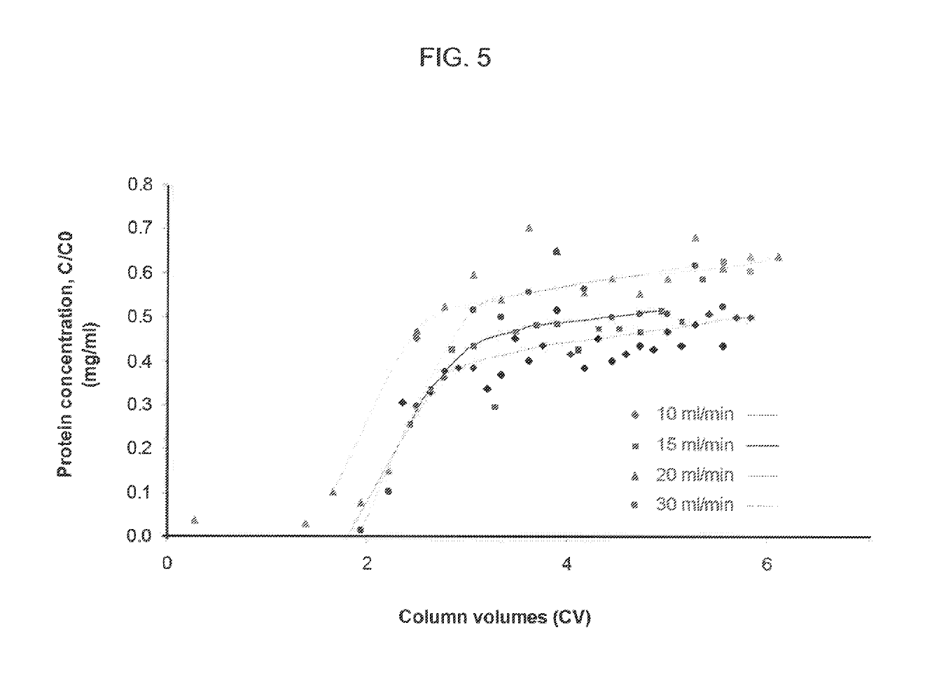

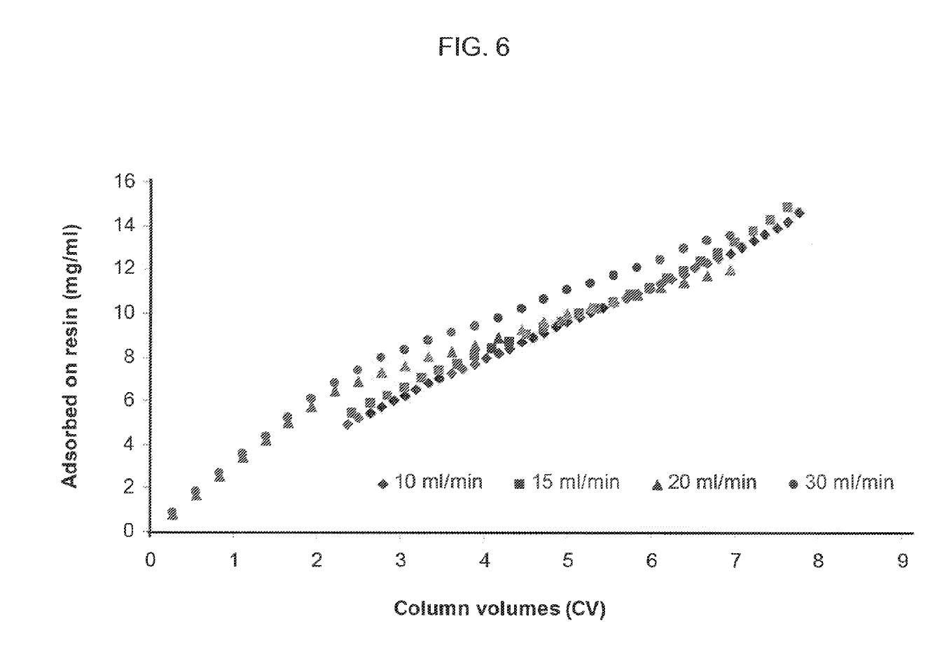

The flow through protein concentration data at three different linear velocity rates were plotted against the column volumes of soy whey loaded (see FIG. 5). These data indicated that increasing the linear flow rate of loading by a factor of 3 resulted in about 10% increase in the unabsorbed proteins in the flow through after loading 6 column volumes of soy whey. Therefore the linear flow rate does not significantly impact the adsorption characteristics of the soy whey proteins with the SP Gibco resin. The equilibrium adsorption data (see FIG. 6) showed that the soy whey protein adsorbed on the resin (calculated using mass balance of protein feed to the system and the protein concentration in the flow through, in equilibrium with the protein in the liquid stream, and plotted against the column volumes passed through the resin bed) varied little with flow rate of the feed at the fluxes tested.

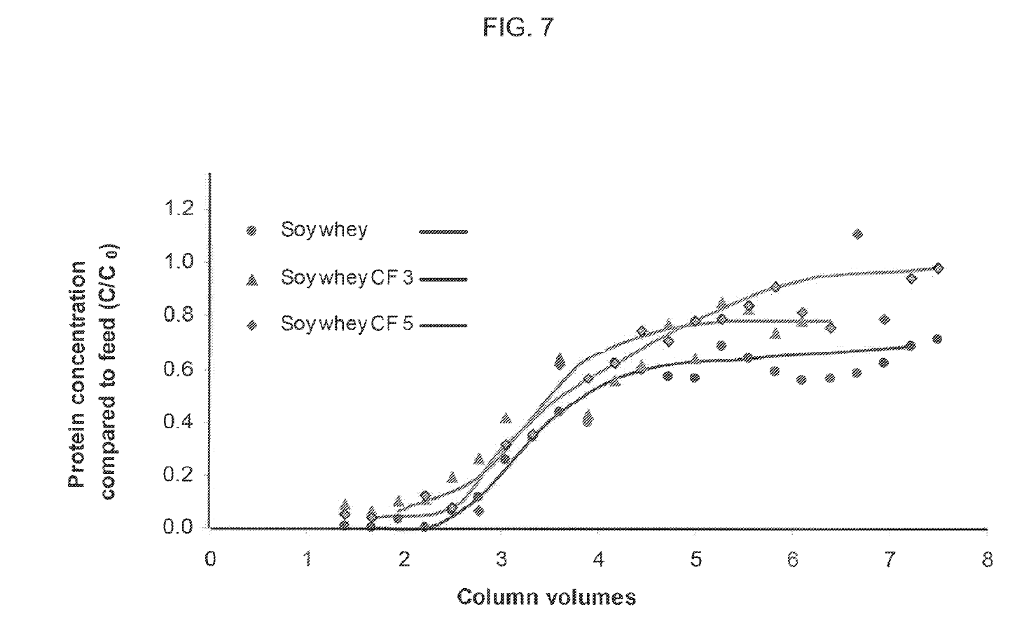

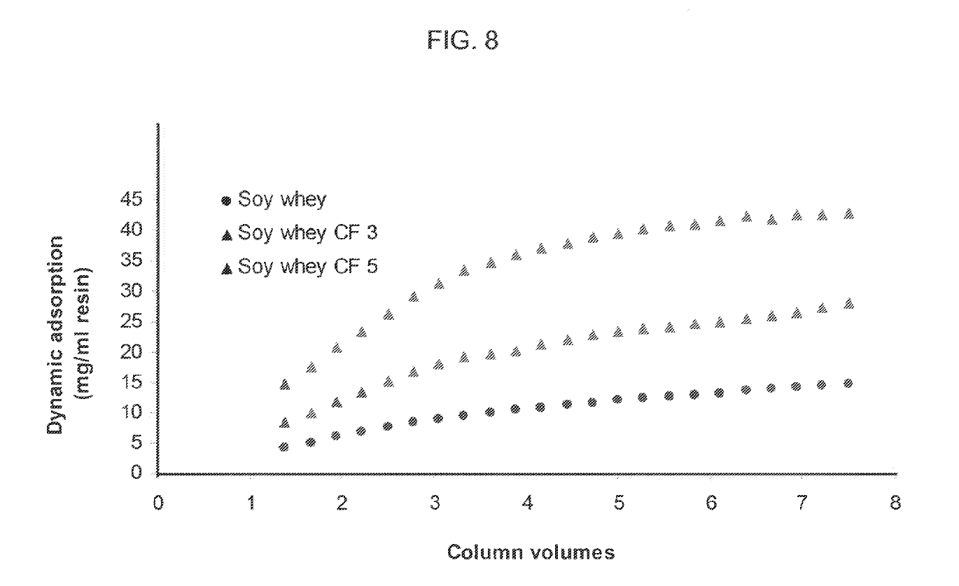

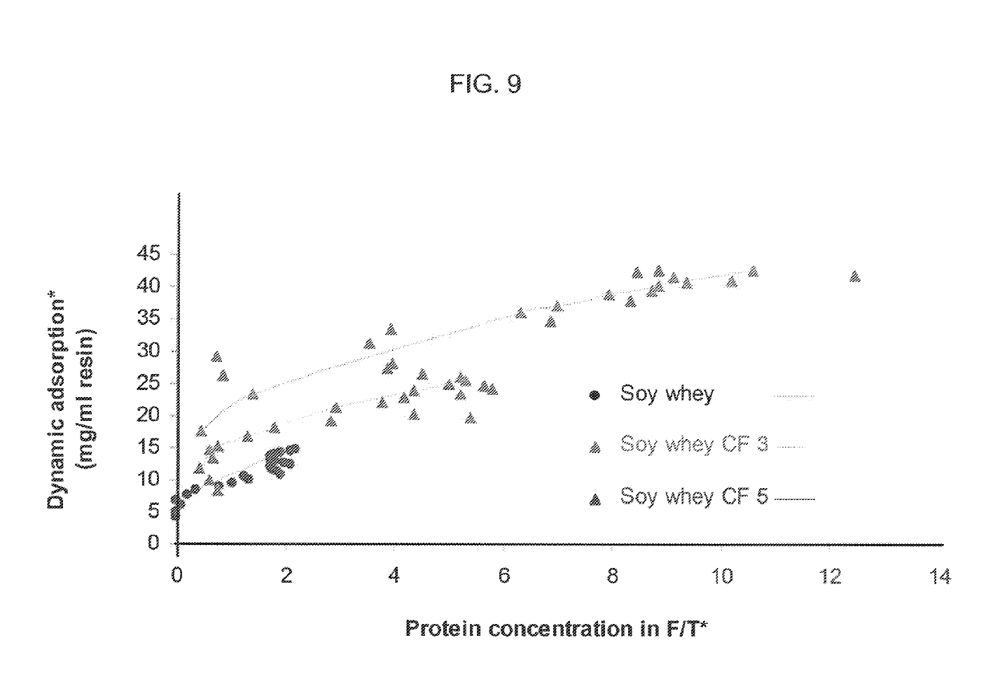

The profile of the breakthrough curve, where soy whey and soy whey concentrated by a factor of 3 and 5 was applied to an SP Gibco resin bed at 15 mL/min (8.5 cm/min linear flow rate), was similar with all three concentrations (see FIG. 7). This result indicated that as the feed protein concentration was increased the resin reached equilibrium with the protein concentration in the liquid stream by striving to reach maximum capacity. This increased adsorption is depicted in FIG. 8 where the protein concentration in the solid phase in equilibrium with the liquid phase has been plotted against the column volumes of soy whey passed through the bed. These data show that the protein adsorbed by the resin significantly increased with soy whey concentration factor, and hence the protein concentration in the soy whey (see FIG. 8). FIG. 9 shows the equilibrium characteristics of the resin and the flow through. This chart shows that as the number of column volumes were passed through the bed, the adsorption of proteins in the resin phase increased asymptotically but the protein content in the flow through also increased. Adsorption capacity can be increased by using concentrated whey and loading at high column volumes but this resulted in a relatively high protein content in the flow through. However, the high protein content in the flow through was minimized by counter current operation using a 2-stage adsorption strategy.

Based on the dynamic adsorption data (see FIG. 9), loading whey concentrated by factor >5 to achieve a protein concentration of >11 mg/mL and loading about 3.5 column volumes resulted in about 35 mg protein adsorbed per mL of resin, and the equilibrium protein concentration in the flow through was about 6.8 mg/mL. Presenting this primary flow through to another resin bed in a second pass (loading about 3.5 column volumes) resulted in a protein concentration in the flow through of about 1.3 mg/mL. Therefore, using two passes of adsorption and operating the chromatography in counter current mode resulted in adsorption of about 90% of the available soy protein that could be absorbed from soy whey at pH 4.5.

Elution Flow Rate

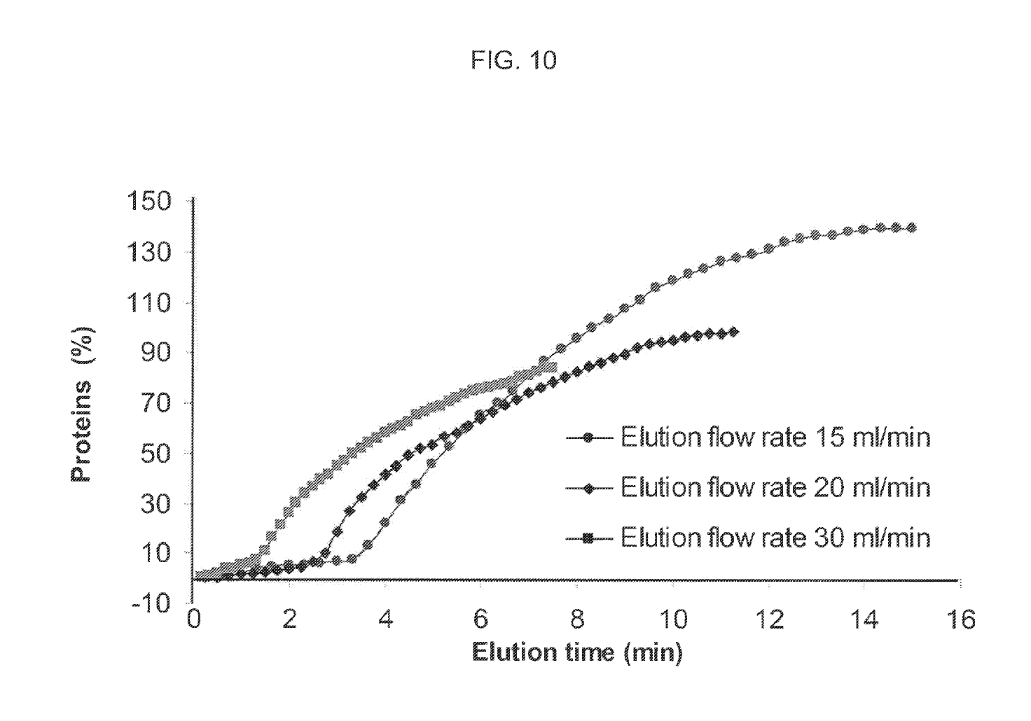

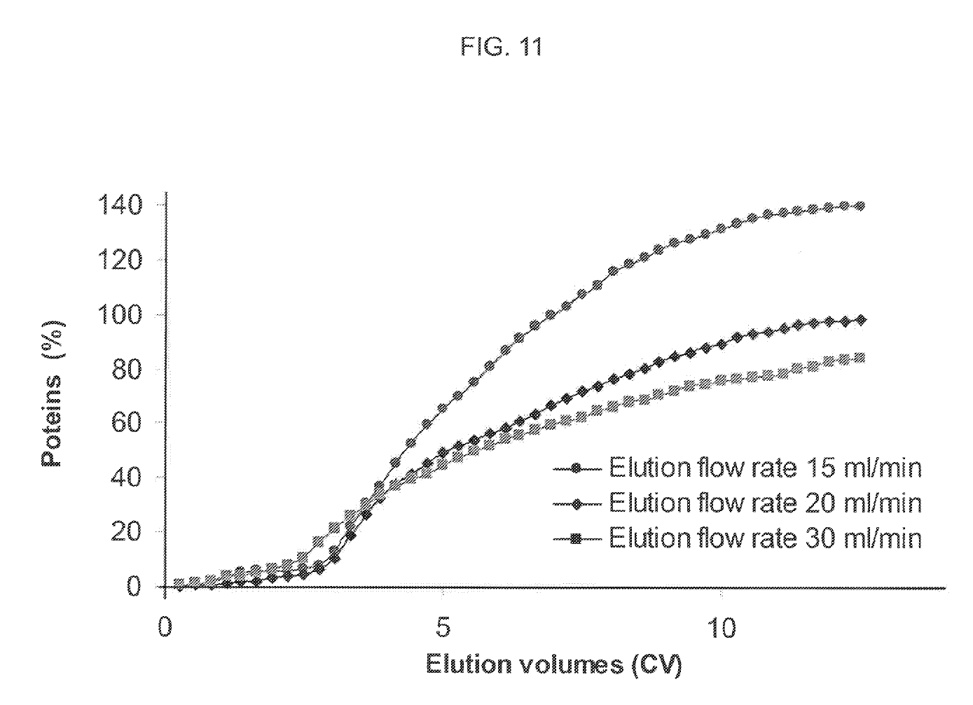

The effect of elution flow rate was investigated at three different flow rates and the recovery data are shown in Table 3. The recovery of protein at low flow rates in duplicate experiments resulted in recoveries of over 164% and 200%. The data indicate that eluting at 20 and 30 mL/min (11.3 and 17.0 cm/min, respectively) did not significantly affect the recoveries. Moreover, operating at higher flow rates achieved much faster elution (see FIG. 10), however at these higher flow rates a larger column volume of eluate was required to complete the elution (see FIG. 11). The need for a larger column volume of eluate was overcome by recycling the eluate which also reduced the total volume required for elution and also presented a more concentrated protein stream to the downstream ultrafiltration unit, reducing the membrane area needed for protein concentration.

TABLE 3
Elution and recovery of bound soy whey
proteins at three different flow rates.

| | ELUTION FLOW RATES | | |
|---|---|---|---|
| | 15 mL/min | 20 mL/min | 30 mL/min |
| Protein adsorbed (mg) | 75.4 ± 4.4 | 70.8 ± 2.7 | 72.9 ± 4.8 |
| Protein eluted (mg) | 139.7 ± 22.9 | 73.2 ± 1.5 | 68.4 ± 6.8 |
| Recovery (%) | 184.2 ± 19.7 | 103.4 ± 6.1 | 93.8 ± 15.6 |

Protein adsorption was calculated as the difference in the protein content in the feed and flow through by mass balance.

### Example 5

### Capture of Bulk Soy Whey Protein From a Pre-Treated Whey Process (PT)

The feed stream to the process, pre-treated whey protein, (also referred to PT whey) had approximately 1.4%-2.0% solids. It was comprised of approximately 18% minerals, 18% protein, and 74% sugars and other materials. Implementation of a Nanofiltration (NF) process allowed for water removal while retaining most of the sugars and protein, and other solid material, in the process to be recovered downstream. The NF membranes (Alfa Laval NF99 8038/48) for the trial were polyamide type thin film composite on polyester membranes with a 2 kDa molecular weight cutoff (MWCO) that allowed water, monovalent cations, and a very small amount of sugars and protein to pass through the pores. The membrane housing held 3 membrane elements. Each element was 8 inches in diameter and had 26.4 square meters of membrane surface area. The total membrane surface area for the process was 79.2 square meters. These membranes were stable up to 1 bar of pressure drop across each membrane element. For the entire module containing 3 membrane elements, a pressure drop of 3 bar was the maximum allowable. The NF feed rate of PT whey was approximately 2,500 L/hour. The temperature of this feed was approximately 45-50° C., and the temperature of the NF operation was regulated to be in this range using cooling water. Initial product flux rates were approximately 16-22 liters per meter squared per hour (LMH). The feed pressure at the inlet of the module was approximately 6 bar. Through the duration of the 6 hour run, the flux dropped as a result of fouling. The feed pressure was increased incrementally to maintain higher flux, but as fouling occurred, the pressure was increased to the maximum, and the flux slowly tapered from that point. Volumetric concentration factors were between 2× and approximately 4×.

A Precipitation step was performed to separate, e.g., phosphorous and calcium salts and complexes from the PT whey. Precipitation conditions were at pH 9 while maintaining the temperature at 45° C. with a residence time of approximately 15 minutes. The precipitation process occurred in a 1000 liter. This tank had multiple inlets and outlets where materials can be piped into and out of it. A small centrifugal pump circulated product out of the tank and back into the side of the tank to promote agitation and effective mixing of the 35% NaOH added to the system to maintain the target pH. This pump also sent product into the centrifuge when one of the T-valves connected to this recirculation loop was opened. Concentrated PT whey from the NF was fed directly into the top of the tank. 35% NaOH was connected into the feed line from the NF in order to control the pH at the target value. PT whey was fed into this mixing tank at approximately 2,500 L/hour and fed out at the same rate.

In following process step, an Alfa Laval Disc Centrifuge (Clara 80) with intermittent solids ejection system was used to separate precipitated solids (including insoluble soy fiber, insoluble soy protein) from the rest of the sugar-and protein-containing whey stream. In this process, concentrated PT whey from the precipitation tank was pumped into a disc-centrifuge where this suspension was rotated and accelerated by centrifugal force. The heavier fraction (precipitated solids) settles on the walls of the rotating centrifuge bowl with the lighter fraction (soluble liquid) was clarified through the use of disc-stacks and continuously discharged for the next step of the process. The separated precipitated solids was discharged at a regular interval (typically between 1 and 10 minutes). The clarified whey stream was less than 0.2% solids on a volumetric basis. The continuous feed flow rate was approximately 2.5 m3/hr, with a pH of 9.0 and 45° C. The insoluble fraction reached Ash=30-60%; Na=0.5-1.5% dry basis, K=1.5-3% dry basis, Ca=6-9% dry basis, Mg=3-6% dry basis, P=10-15% dry basis, Cl=1-2% dry basis, Fe, Mn, Zn, Cu<0.15% dry basis. Changes to the soluble fraction were as follows: Phytic acid was approximately 0.3% dry basis (85% reduction, P=0.2-0.3% dry basis (85-90% reduction), Ca=0.35-0.45% dry basis (80-85% reduction), Mg=0.75-0.85% dry basis (15-20% reduction).

The next step was an Ultrafiltration (UF) membrane. Protein was concentrated by being retained by a membrane while other smaller solutes pass into the permeated stream. From the centrifuge a diluted stream the containing protein, minerals and sugars was fed to the UF. The UF equipment and the membrane were supplied from Alfa Laval while the ClP chemicals came from Ecolab, Inc. The tested membrane, GR70PP/80 from Alfa-Laval, had a MWCO of 10 kD and was constructed of polyethersulfone (PES) cast onto a polypropylene polymer backing. The feed pressure varied throughout the trial from 1-7 bar, depending upon the degree of fouling of the membranes. The temperature was controlled to approximately 65° C. The system was a feed and bleed setup, where the retentate was recycled back to the feed tank while the permeate proceeded on to the next step in the process. The system was operated until a volume concentration factor of 30× was reached. The feed rate to the UF was approximately 1,600 L/hour. The setup had the ability to house 3 tubes worth of 6.3″ membrane elements. However, only one of the three tubes was used. The membrane skid had an automatic control system that allowed control of the temperature, operating pressures (inlet, outlet, and differential) and volume concentration factor during process. Once the process reached the target volume concentration factor, typically after 6-8 hours of operation, the retentate was diafiltered (DF) with one cubic meter of water, (approximately 5 parts of diafiltration water per part of concentrated retentate) to yield a high protein retentate. After a processing cycle, the system was cleaned with a typical CIP protocol used with most protein purification processes. The retentate contained about 80% dry basis protein after diafiltration.

The permeate of the UF/DF steps contained the sugars and was further concentrated in a Reverse Osmosis Membrane system (RO). The UF permeate was transferred to an RO system to concentrate the feed stream from approximately 2% total solids (TS) to 20% TS. The process equipment and membranes (RO98pHt) for the RO unit operation were supplied by Alfa-Laval. The feed pressure was increased in order to maintain a constant flux, up to 45 bar at a temperature of 50° C. Typically each batch started at a 2-3% Brix and end at 20-25% Brix (Brix=sugar concentration).

After the RO step the concentrated sugar stream was fed to an Electrodialysis Membrane (ED). Electrodialysis from Eurodia Industrie SA removes minerals from the sugar solution. The electrodialysis process has two product streams. One is the product, or diluate, stream which was further processed to concentrate and pasteurize the SOS concentrate solution. The other stream from the electrodialysis process is a brine solution which contains the minerals that were removed from the feed stream. The trial achieved >80% reduction in conductivity, resulting in a product stream that measured <3 mS/cm conductivity. The batch feed volume was approx 40 liters at a temperature of 40° C. and a pH of 7. The ED unit operated at 18V and had up to 50 cells as a stack size.

The de-mineralized sugar stream from the ED was further processed in an Evaporation step. The evaporation of the SOS stream was carried out on Anhydro's Lab E vacuum evaporator. SOS product was evaporated to 40-75% dry matter with a boiling temperature of approximately 50-55° C. and a AT of 5-20° C.

A Spray Dryer was used to dry UF/DF retentate suspension. The UF diafiltrate retentate, with a solids content of approximately 8%, was kept stirred in a tank. . The suspension was then fed directly to the spray dryer where it was combined with heated air under pressure and then sprayed through a nozzle. The dryer removed the water from the suspension and generated a dry powder, which was collected in a bucket after it was separated from the air stream in a cyclone. The feed suspension was thermally treated at 150° C. for 9 seconds before it entered the spray dryer to kill the microbiological organisms. The spray dryer was a Production Minor from the company Niro/GEA. The dryer was set up with co-current flow and a two fluid nozzle. The drying conditions varied somewhat during the trial. Feed temperatures were about 80° C., nozzle pressure was about 4 bars, and inlet air temperatures was about 250° C.

### Example 6

### Capture of Bulk Soy Whey Protein Whey Pre-Treatment Process and Cross-Flow Filtration Membranes

Approximately 8000 lbs of aqueous soy whey (also referred to as raw whey) at 110° F. and 4.57 pH from an isolated soy protein extraction and isoelectric precipitation continuous process was fed to a reaction vessel where the pH was increased to 5.3 by the addition of 50% sodium hydroxide. The pH-adjusted raw whey was then fed to a second reaction vessel with a 10 minute average residence time in a continuous process where the temperature was increased to 190° F. by the direct injection of steam. The heated and pH-adjusted raw whey was then cooled to 90 degrees F. by passing through a plate and frame heat exchanger with chilled water as the cooling medium. The cooled raw whey was then fed into an Alfa Laval VNPX510 clarifying centrifuge where the suspended solids, predominantly insoluble large molecular weight proteins, were separated and discharged in the underflow to waste and the clarified centrate proceeded to the next reaction vessel. The pH of the clarified centrate, or pre-treated whey protein, was adjusted to 8.0 using 12.5% sodium hydroxide and held for 10 minutes prior to being fed into an Alfa Laval VNPX510 clarifying centrifuge where the suspended solids, predominantly insoluble minerals, were separated and discharged in the underflow to waste. The clarified centrate proceeded to a surge tank prior to ultrafiltration. Ultrafiltration of the clarified centrate proceeded in a feed and bleed mode at 90° F. using 3.8″ diameter polyethersulfone spiral membranes, PW3838C, made by GE Osmonics, with a 10 kDa molecular weight cut-off. Ultrafiltration continued until a 60× concentration of the initial feed volume was accomplished, which required about 4.5 hrs. The retentate, 114 lbs at 4.5% total solids and 8.2 pH, was transferred to a reaction vessel where the pH was adjusted to 7.4 using 35% hydrochloric acid. The retentate was then heated to 305° F. for 9 seconds via direct steam injection prior to flash cooling to 140° F. in a vacuum chamber. The material was then homogenized by pumping through a homogenizing valve at 6000 psi inlet and 2500 outlet pressure prior to entering the spray drier through a nozzle and orifice combination in order to atomize the solution. The spray drier was operated at 538° F. inlet temperature and 197° F. outlet temperature, and consisted of a drying chamber, cyclone and baghouse. The spray dried soy whey protein, a total of 4 lbs, was collected from the cyclone bottom discharge.

### Example 7

### Capture of Bulk Soy Whey Protein Using Expanded Bed Adsorption (EBA) Chromatography

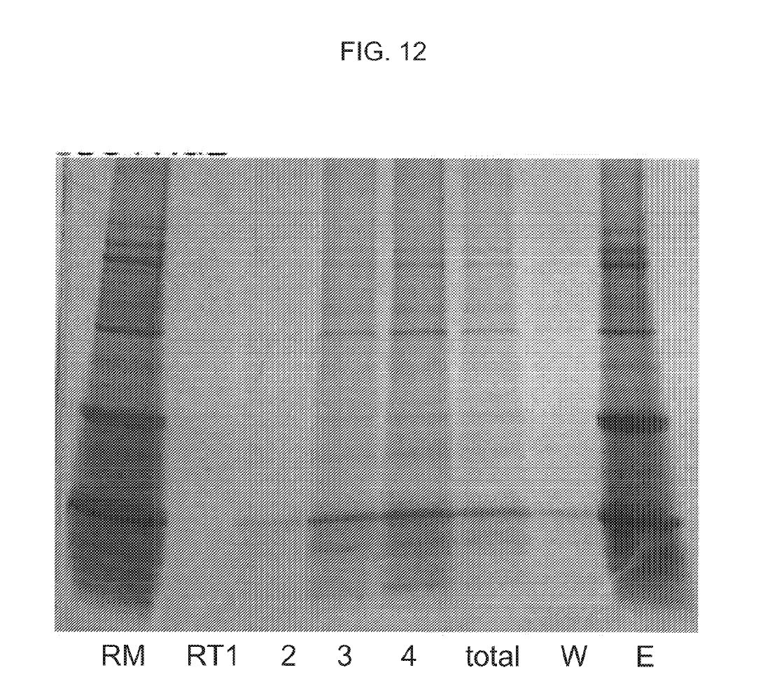

200 ml of aqueous raw soy whey (not pre-treated) with a total solids content of 1.92%, was adjusted to pH 4.5 with acetic acid and applied to a 1×25 cm column of Mimo6ME resin (UpFront Chromatography, Copenhagen Denmark) equilibrated in 10 mM sodium citrate, pH 4.5. Material was loaded onto the column from the bottom up at 20-25° C. using a linear flow rate of 7.5 cm/min. Samples of the column flow-through were collected at regular intervals for later analysis. Unbound material was washed free of the column with 10 column volumes of equilibration buffer, then bound material recovered by elution with 50 mM sodium hydroxide. 10 μls of each fraction recovered during EBA chromatography of aqueous soy whey were separated on a 4-12% SDS-PAGE gel and stained with Coomassie Brilliant Blue R 250 stain. SDS-PAGE analysis of the column load, flow-through, wash, and sodium hydroxide eluate samples is depicted in FIG. 12. As used in FIG. 12, RM: raw material (column load); RT1-4: column flow-through (run through) collected at equal intervals during the load; total: the total run-through fraction; W: column wash; E: column eluate. Binding was reasonably efficient, as very little protein is seen in the initial breakthrough fractions, only showing up in the later fractions. A total of 662 mg of protein was recovered in the eluate, for a yield of 3.3 mg/ml of starting material. Under these conditions, the capacity of this resin was shown to be 33.1 mg of protein per ml of adsorbent.

### Example 8

### Capture of Bulk Soy Whey Protein From Spray-Dried SWP Using Expanded Bed Adsorption (EBA) Chromatography

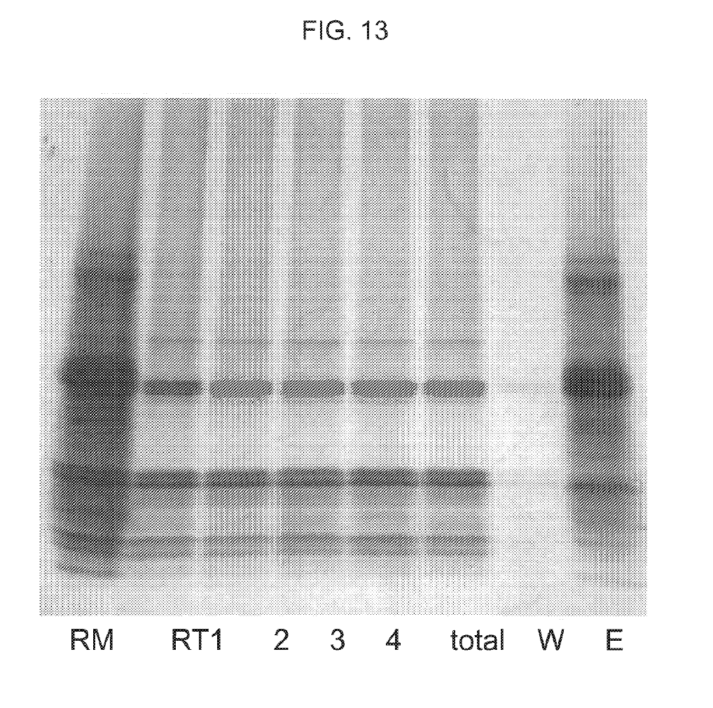

Spray-dried soy whey powder was slurried to a concentration of 10 mg/ml in water and adjusted to pH 4.0 with acetic acid. 400 ml of the slurry was then applied directly to the bottom of a 1×25 cm column of Mimo-4SE resin (UpFront Chromatography, Copenhagen Denmark) that had been equilibrated in 10 mM sodium citrate, pH 4.0. Material was loaded at 20-25° C. using a linear flow rate of 7.5 cm/min. Samples of the column flow-through were collected at regular intervals for later analysis. Unbound material was washed free of the column using 10 column volumes of equilibration buffer. Bound material was eluted with 30 mM NaOH. 10 μls of each fraction recovered during EBA chromatography of a suspension of soy whey powder were separated on a 4-12% SDS-PAGE gel and stained with Coomassie Brilliant Blue R 250 stain. SDS-PAGE analysis of the column load, flow-thru, wash, and eluate are depicted in FIG. 13. As used in FIG. 13, RM: raw material (column load); RT1-4: column flow-through (run through) collected at equal intervals during the load; total: the total run-through fraction; W: column wash; E: column eluate. Binding was not as efficient as was observed using the Mimo6ME resin, as several protein bands are seen in the breakthrough fractions. A total of 2070 mg of protein were recovered in the eluate, for a yield of 5.2 mg/ml of starting material. Under these conditions, the capacity of this resin was shown to be 104 mg of protein per ml of adsorbent.

### Example 9

### Removal of KTI From Bulk Soy Whey Protein Using Expanded Bed Adsorption (EBA) Chromatography

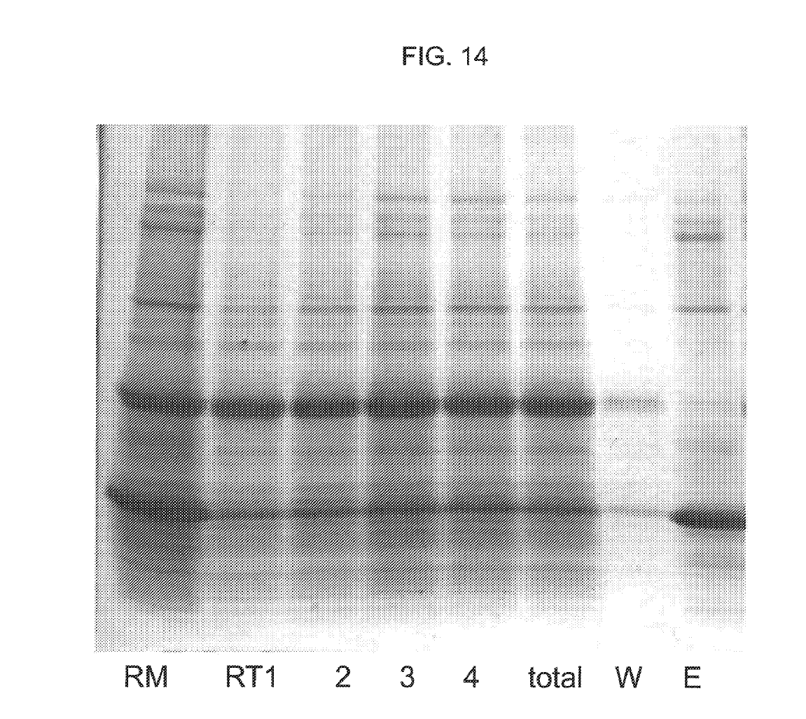

Two procedures were used to remove the majority of contaminating KTI protein from the bulk of the soy whey protein by EBA chromatography. In the first, 200 ml of aqueous raw soy whey (not pre-treated) with a total solids content of 1.92%, was adjusted to pH 6.0 with sodium hydroxide and applied to a 1×25 cm column of Mimo6HE resin (UpFront Chromatography, Copenhagen Denmark) equilibrated in10 mM sodium citrate, pH 6.0. Material was loaded onto the column from the bottom up at 20-25° C. using a linear flow rate of 7.5 cm/min. Samples of column flow-through were collected at regular intervals for later analysis. Unbound material was washed free of the column with 10 column volumes of equilibration buffer, then bound material recovered by elution with 30 mM sodium hydroxide. 10 μls of each fraction recovered during EBA chromatography of a suspension of soy whey powder were separated on a 4-12% SDS-PAGE gel and stained with Coomassie Brilliant Blue R 250 stain. SDS-PAGE analysis of the column load, flow-through, wash, and sodium hydroxide eluate samples is depicted in FIG. 14. As used in FIG. 14, RM: raw material (column load); RT1-4: flow-through material (run through) collected at equal intervals during the load; total: the total run-through fraction; W: column wash; E: column eluate. The bulk of the loaded protein is clearly seen eluting in the flow-through, while the bulk of the KTI protein remains bound to the resin. A total of 355 mg of protein, the bulk of which is KTI, was recovered in the eluate, for a yield of 1.8 mg/ml of starting material. Under these conditions, the capacity of this resin was shown to be 17.8 mg of KTI (plus minor contaminants) per ml of adsorbent.

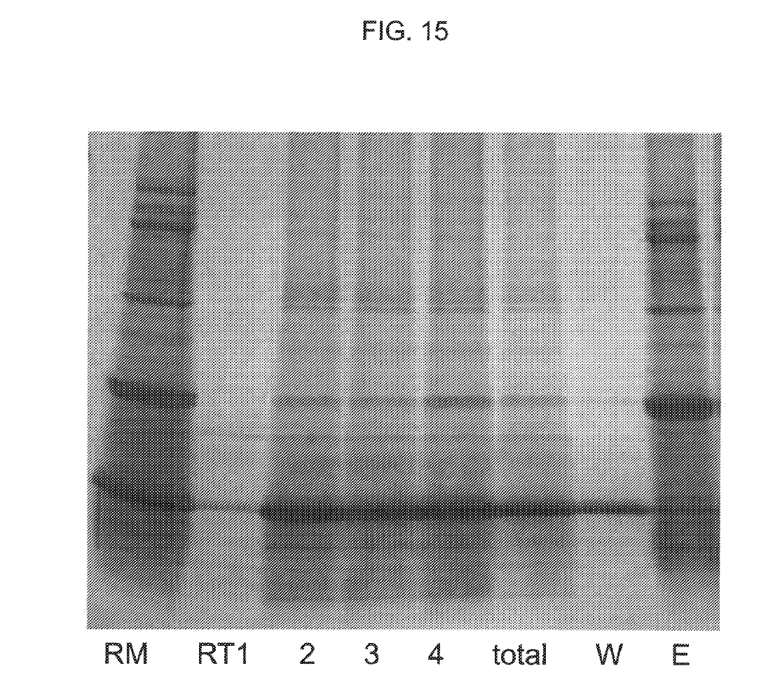

In the second procedure, 160 mls of aqueous raw soy whey (not pre-treated) with a total solids content of 1.92%, was adjusted to pH 5.1 with acetic acid and applied to a 1×25 cm column of Mimo6ZE resin (UpFront Chromatography, Copenhagen Denmark) equilibrated in 10 mM sodium citrate, pH 5.0. Material was loaded onto the column from the bottom up at 20-25° C. using a linear flow rate of 7.5 cm/min. Samples of column flow-through were collected at regular intervals for later analysis. Unbound material was washed free of the column with 10 column volumes of equilibration buffer, then bound material recovered by elution with 30 mM sodium hydroxide. 10 μls of each fraction recovered during EBA chromatography of a suspension of soy whey powder were separated on a 4-12% SDS-PAGE gel and stained with Coomassie Brilliant Blue R 250 stain. SDS-PAGE analysis of the column load, flow-through, wash, and sodium hydroxide eluate samples is depicted in FIG. 15. As used in FIG. 15, RM: raw material (column load); RT1-4: flow-through material (run through) collected at equal intervals during the load; total: the total run-through fraction; W: column wash; E: column eluate. The bulk of the KTI is clearly seen eluting in the flow-through, while the bulk of the remaining protein remains bound to the resin. A total of 355 mg of soy protein essentially devoid of contaminating KTI was recovered in the eluate, for a yield of 2.1 mg/ml of starting material. Under these conditions, the capacity of this resin was shown to be 16.8 mg of soy protein per ml of adsorbent.

### Example 10

### Formation of Flame Retardant Foam Containing a Foaming Agent Comprising an Amount of Soy Whey Protein

A flame retardant foam product can be prepared according to typical industry processing techniques using a foaming agent from soy whey protein as described hereinabove. Table 4 is the list of ingredients that can be used to prepare a flame retardant foam product having a foaming agent comprised of between 1.0%-10.0% soy whey protein.

TABLE 4
Flame Retardant Foam Formulation with a Foaming
Agent Comprised of Soy Whey Protein

| Ingredient | Concentration (wt %) |
|---|---|
| Water | 85.0-95.0% |
| Soy Whey Protein | 1.0-10.0% |
| Ethylene Glycol; 1,2 Ethanediol | 1.0-2.0% |
| Hexylene Glycol; 2 methyl-2,4 Pentanediol | 1.0-2.0% |
| Ferrous Sulfate | 0.1-1.0% |
| Zinc Chloride | 0.5-3.0% |
| TOTAL | To make 100% |

The flame retardant foam samples that can be prepared with a foaming agent comprising a low amount of soy whey protein (i.e., 1.0% -10.0% soy whey protein) will produce stable and sustainable foam similar to a flame retardant foam containing only commonly known surfactants as foaming agents.

### Example 11

### Formation of a Hand Cleanser Containing a Foaming Agent Comprising an Amount of Soy Whey Protein

A hand cleanser product was prepared according to typical industry processing techniques using a foaming agent comprised of soy whey protein as described hereinabove. Table 5 is the list of ingredients used to prepare a hand cleanser product having a foaming agent comprised of soy whey protein.

TABLE 5
Hand Cleanser Formulation with a Foaming
Agent Comprised of Soy Whey Protein

| | Concentration | |
|---|---|---|
| Ingredient | (wt %) | Grams |
| Lactic acid (Purac ® Hipure90) | 0.20 | 0.80 |
| Cocamidopr(Tego ® Betain C60) | 5.00 | 20.00 |
| Ammonium Laureth Sulfate | 15.00 | 60.00 |
| Ammonium Lauryl Sulfate | 15.00 | 60.00 |
| Soy Whey Protein | 10.00 | 40.00 |
| Sodium benzoate, Potassium sorbate | 1.00 | 4.00 |
| (Euxyl ® K 712) (preservative) | | |
| 20% NaCl Aq. solution | 0.28 | 1.12 |
| Water | 53.52 | 214.08 |
| TOTAL | 100.00% | 400.00 grams |

The hand cleanser product was prepared by first charging the water into a main vessel. The soy whey protein was slowly sifted into the water and the protein slurry was mixed until uniform. The protein slurry was then heated to a temperature of about 75° C. while mixing continued and until the protein was fully hydrated. Mixing continued while the slurry was beginning to cool. The ammonium lauryl sulfate and ammonium laureth sulfate were added to the main vessel while mixing. The remaining components were then added in the following order while mixing the contents to achieve room temperature: Tego® Betain C60, Purac® Hipure90, Euxyl® K 712, and 20% NaCl aqueous solution. The contents were mixed after each addition in order to achieve uniformity.

Depending on the type of dispenser chosen for the hand cleanser, the final product may either be in liquid form or foam form. For instance, the hand cleanser could be supplied in a charged container or foaming pump container whereby the cleanser would foam immediately upon release from the container by the user. Alternatively, the liquid cleanser could be provided in a non-charged container or non-foaming pump dispenser such that the cleanser would remain in liquid form when dispensed from the container and would only foam upon reaction with water.

The hand cleanser prepared with a foaming agent comprising a low amount of soy whey protein (i.e., 10% soy whey protein) produced a stable and sustainable foam when used similar to a hand cleanser containing only commonly known surfactants as foaming agents.

### Example 12

### Formation of a Toothpaste Product Containing a Foaming Agent Comprised of an Amount of Soy Whey Protein

A toothpaste product was prepared according to typical industry processing techniques using a foaming agent comprised of soy whey protein as described hereinabove. Table 6 is the list of ingredients that were used to prepare a toothpaste product having a foaming agent comprised of soy whey protein.

TABLE 6
Toothpaste Formulation with a Foaming
Agent Comprised of Soy Whey Protein

| | Concentration | |
|---|---|---|
| Ingredient | (wt %) | Grams |
| Glycerin | 25.00 | 125.00 |
| Carageenan (Ticagel ® 795) | 0.60 | 3.00 |
| Calcium Carbonate | 25.00 | 125.00 |
| (Acala ™ USP 7300) | | |
| Calcium Carbonate | 15.00 | 75.00 |
| (Acala ™ USP 5300) | | |
| Calcium Carbonate | 15.00 | 75.00 |
| (Microwhite ® Codex 100) | | |
| Soy Whey Protein | 5.70 | 28.50 |
| Mica 100K | 2.00 | 10.00 |
| Peppermint oil | 0.80 | 4.00 |
| Stevia leaf powder | 0.20 | 1.00 |
| Water | 10.70 | 53.50 |
| TOTAL | 100.00% | 500.00 grams |

The toothpaste product was prepared by first premixing the glycerin and Ticagel® 795 in a main vessel. When uniform, the water was slowly added. Slow mixing of the blend continued until uniform.

In a separate vessel, the Acala™ USP 7300, Acala™ USP5300, Microwhite® Codex 100, Mica 100 K, and Stevia leaf powder were premilled together. The powder blend was then slowly sifted into the main vessel under slow agitation. Slow mixing of the blend continued until the contents were uniform.

In a separate vessel, the soy whey protein and peppermint oil were mixed together and then slowly added to the main vessel avoiding aeration. The contents were mixed until uniformity was achieved.

The toothpaste prepared with a foaming agent comprising a low amount of soy whey protein (i.e., 5.70% soy whey protein) produced a stable and sustainable foam when used similar to a toothpaste containing only commonly known surfactants as foaming agents.

### Example 13

### Formation of a Powder Detergent Containing a Foaming Agent Comprising an Amount of Soy Whey Protein

A powder detergent product can be prepared according to typical industry processing techniques using a foaming agent comprised of soy whey protein as described hereinabove. Table 7 is the list of ingredients that can be used to prepare a powder detergent product having a foaming agent comprised of soy whey protein.

TABLE 7
Powder Detergent Formulation with a Foaming
Agent Comprised of Soy Whey Protein

| Ingredient | Conc. (wt %) |
|---|---|
| Linear Alkylbenzenesulfonate (LAS) | 20 |
| Soy Whey Protein (SWP) | 5 |
| Sodium Tripolyphosphate (STPP) | 25 |
| Sodium Carbonate | 5 |
| Sodium Sulfate | 35 |
| Polyacrylate | 1 |
| Bleach and Bleach Activators | 7 |
| Detersive Enzymes | 2 |
| TOTAL | 100% |

The powder detergent samples that can be prepared with a foaming agent comprising a low amount of soy whey protein (i.e., 5% soy whey protein) will produce stable and sustainable foam when used similar to a powder detergent containing only commonly known surfactants as foaming agents.

### Example 14

### Formation of a Shaving Cream Containing a Foaming Agent Comprising an Amount of Soy Whey Protein

A shaving cream product was prepared according to typical industry processing techniques using a foaming agent comprised of soy whey protein as described hereinabove. Table 8 is the list of ingredients that were used to prepare a shaving cream having a foaming agent comprised of soy whey protein.

TABLE 8
Shaving Cream Formulation with a Foaming
Agent Comprised of Soy Whey Protein

| | Concentration | |
|---|---|---|
| Ingredient | (wt %) | Grams |
| Soy Whey Protein | 10.00 | 40.00 |
| Soybean oil | 20.00 | 80.00 |
| Jojoba oil | 10.00 | 40.00 |
| Argan oil | 2.00 | 8.00 |
| Avocado oil | 7.00 | 28.00 |
| Coconut oil | 10.00 | 40.00 |
| Vitamin E Acetate | 0.100 | 0.400 |
| Acrylates/C10-30 Alkyl Acrylate | 0.70 | 2.80 |
| Crosspolymer (Carbopol ® Ultrez 20) | | |
| Sodium Benzoate, Potassium | 1.00 | 4.00 |
| sorbate (Euxyl ® K 712) | | |
| Water | 39.20 | 156.80 |
| TOTAL | 100.00% | 400.00 grams |

The shaving cream was prepared by first charging the water in a main vessel and applying moderate shear. The soy whey protein was added to the water and the slurry was mixed to uniformity. In a separate vessel, the oils and vitamin E acetate were combined and mixed to uniformity. The oil mixture was warmed slightly as needed to keep the oils in liquid form. The Carbopol® Ultrez 20 was dispersed into the oil mixture and mixing continued until all of the components were uniform.

The oil mixture containing the Carbopol® Ultrez 20 was then added to the protein slurry in the main vessel under moderate agitation. The mixture was homogenized to uniformity.

After completing the homogenization step, the Euxyl® K 712 was added to the mixture and the contents were mixed with an impeller until uniform.

The shaving cream prepared with a foaming agent comprising a low amount of soy whey protein (i.e., 10% soy whey protein) produced a stable and sustainable foam when used similar to a shaving cream containing only commonly known surfactants as foaming agents.

### Example 15

### Formation of a Make-up Remover Containing a Foaming Agent Comprising an Amount of Soy Whey Protein

A make-up remover/facial cleansing product was prepared according to typical industry processing techniques using a foaming agent comprised of soy whey protein as described hereinabove. Table 9 is the list of ingredients that were used to prepare a make-up remover/facial cleanser having a foaming agent comprised of soy whey protein.

TABLE 9
Make-up Removing Formulation with a Foaming
Agent Comprised of Soy Whey Protein

| | Concentration | |
|---|---|---|
| Ingredient | (wt %) | Grams |
| Soy Whey Protein | 5.00 | 20.00 |
| Xanthan gum | 0.30 | 1.20 |
| Sodium benzoate, Potassium | 5.00 | 20.00 |
| sorbate (Euxyl ® K 712) | | |
| Water | 93.70 | 374.80 |
| TOTAL | 100.00% | 400.00 grams |

The make-up remover/facial cleanser was prepared by first charging the water in a main vessel. The xanthan gum was added and the contents were mixed to uniformity. The soy whey protein was then added to the main vessel and mixing continued until uniform. The Euxyl® K 712 was added to the mixture and the contents were mixed again until uniform.

The make-up remover/facial cleanser prepared with a foaming agent comprising a low amount of soy whey protein (i.e., 5% soy whey protein) produced a stable and sustainable foam when used similar to a facial cleanser containing only commonly known surfactants as foaming agents.

### Example 16

### Formation of a Facial Cleanser Containing a Foaming Agent Comprising an Amount of Soy Whey Protein

A facial cleanser product was prepared according to typical industry processing techniques using a foaming agent comprised of soy whey protein as described hereinabove. Table 10 is the list of ingredients that were used to prepare a facial cleanser lotion product having a foaming agent comprised of soy whey protein.

TABLE 10
Facial Cleanser Formulation with a Foaming
Agent Comprised of Soy Whey Protein

| | Concentration | |
|---|---|---|
| Ingredient | (wt %) | Grams |
| Soy Whey Protein | 10.00 | 40.00 |
| Poloxamer 407 | 10.00 | 40.00 |
| Blend of | 1.00 | 4.00 |
| cocoamphocarboxyglycinate | | |
| including sodium lauryl sulfate | | |
| PPG-26 oleate (Polypropylene | 1.00 | 4.00 |
| glycol ester of oleic acid) | | |
| Blend of acetylated lanolin | 1.00 | 4.00 |
| alcohol including cetyl | | |
| acetate and polysorbate 80 | | |
| Water | 77.00 | 308.00 |
| TOTAL | 100.00% | 400.00 g |

The facial cleanser was prepared by first charging the amount of water and Poloxamer 407 into a main vessel immersed in an ice water bath. Moderate shear was applied until all of the Poloxamer 407 was dissolved. The soy whey protein was added to the main vessel and mixing continued until the contents were uniformly combined. The blend of cocoamphocarboxyglycinate was added to the main vessel and mixing continued until the contents were uniformly combined. In a separate vessel, the PPG-26 oleate and blend of acetylated lanolin alcohol were combined and the blend was added to the main vessel. The batch was mixed until uniform, while raising the temperature of the batch to room temperature.

The facial cleanser samples that were prepared with a foaming agent comprising a low amount of soy whey protein (i.e., 10% soy whey protein) produced a stable and sustainable emulsion similar to a facial cleanser containing only commonly known surfactants as foaming agents.

Depending on the type of dispenser chosen for the cleanser, the final product may either be in liquid form or foam form. For instance, the cleanser could be provided in a charged container or foaming pump container whereby the cleanser would foam upon release from the container by the user. Alternatively, the liquid could be provided in a non-charged container or non-foaming pump dispenser such that the cleanser would remain in liquid form when dispensed from the container and would only foam upon reaction with water.

### Example 17

### Formation of a Conditioning Hair Shampoo Containing a Foaming Agent Comprising an Amount of Soy Whey Protein

A conditioning hair shampoo was prepared according to typical industry processing techniques using a foaming agent comprised of soy whey protein as described hereinabove. Table 11 is the list of ingredients that were used to prepare a conditioning hair shampoo having a foaming agent comprised of soy whey protein in addition to a commonly known surfactant.

TABLE 11
Conditioning Hair Shampoo Formulation with a
Foaming Agent Comprised of Soy Whey Protein

| | Concentration | |
|---|---|---|
| Ingredient | (wt %) | Grams |
| Disodium Sulfosuccinate | 40.00 | 200.00 |
| Laurylglucoside Crosspolymer | | |
| (Poly Suga ®Mate L) (surfactant) | | |
| Soy whey protein (foaming agent) | 6.00 | 30.00 |
| Glycol Distearate (Cutina ® AGS) | 4.00 | 20.00 |
| Sodium Benzoate, Potassium | 1.00 | 5.00 |
| Sorbate (Euxyl ® K 712) (preservative) | | |
| Fragrance | 0.30 | 1.50 |
| Water | 48.70 | 243.50 |
| TOTAL | 100.00% | 500.00 g |

The conditioning hair shampoo was prepared by first charging the amount of water into a main vessel. Moderate shear was applied while sifting in the soy whey protein. The blend was heated to a temperature of 80° C. while mixing to achieve uniformity. Poly Suga®Mate L and Cutina® AGS were then added to the blend while the temperature of 80° C. was maintained and mixed to achieve complete uniformity. While mixing, the blend was cooled to 40° C. Once the reduced temperature of 40° C. was attained, the fragrance and Euxyl® K 712 were then added. The entire blend was mixed until uniform and until room temperature was attained.

The conditioning hair shampoo samples that were prepared with a foaming agent comprising a low amount of soy whey protein (i.e., 6% soy whey protein) will produce stable and sustainable foam similar to a hair conditioning shampoo formulation containing only commonly known surfactants as foaming agents.

### Example 18

### Determination of SSI

A sample of the protein material is obtained by accurately weighing out 12.5 g of protein material. 487.5 g of deionized water is added to a quart blender jar. 2 to 3 drops of defoamer (Dow Corning® Antifoam B Emulsion, 1:1 dilution with water) is added to the deionized water in the blender jar. The blender jar containing the water and defoamer is placed on a blender (Osterizer), and the blender stirring speed is adjusted to create a moderate vortex (about 14,000 rpm). A timer is set for 90 seconds, and the protein sample is added to the water and defoamer over a period of 30 seconds while blending. Blending is continued for the remaining 60 seconds after addition of the protein sample (total blending time should be 90 seconds from the start of addition of the protein sample).

The resulting protein material sample/water/defoamer slurry is then transferred to a 500 ml beaker containing a magnetic stirring bar. The beaker is then covered with plastic wrap or aluminum foil. The covered beaker containing the slurry is then placed on a stirring plate, and the slurry is stirred at moderate speed for a period of 30 minutes.

200 g of the slurry is then transferred into a centrifuge tube. A second 200 g sample of the slurry is then transferred into a second centrifuge tube. The remaining portion of the slurry in the beaker is retained for measuring total solids. The 2 centrifuge tube samples are then centrifuged at 500×g for 10 minutes (1500 rpm on an IEC Model K). At least 50 ml of the supernatant is withdrawn from each centrifuge tube and placed in a plastic cup (one cup for each sample from each centrifuge tube, 2 total cups).

Soluble Solids is then determined by drying a 5 g sample of each supernatant at 130° C. for 2 hours, measuring the weights of the dried samples, and averaging the weights of the dried samples.

Total Solids is determined by drying two 5 g samples of the slurry retained in the beaker, measuring the weights of the dried samples, and averaging the weights of the dried samples.

The Soluble Solids Index (SSI) is calculated from the Soluble Solids and Total Solids according to the formula (Soluble Solids/Total Solids)×100.

One skilled in the art would readily appreciate that the methods, compositions, and products described herein are representative of exemplary embodiments, and not intended as limitations on the scope of the invention. It will be readily apparent to one skilled in the art that varying substitutions and modifications may be made to the present disclosure disclosed herein without departing from the scope and spirit of the invention.

All patents and publications mentioned in the specification are indicative of the levels of those skilled in the art to which the present disclosure pertains. All patents and publications are herein incorporated by reference to the same extent as if each individual publication was specifically and individually indicated as incorporated by reference.

The present disclosure illustratively described herein suitably may be practiced in the absence of any element or elements, limitation or limitations that are not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the present disclosure claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A foaming agent comprising an amount of soy whey protein.

2. The foaming agent of claim 1, wherein the soy whey protein has a soluble solids index (SSI) of at least about 80% across a pH range of from 2 to 10 and a temperature of 25° C.

3. The foaming agent of claim 1, wherein the foaming agent comprises 100% by weight of soy whey protein.

4. The foaming agent of claim 1, further comprising at least one additional foaming agent.

5. The foaming agent of claim 4, wherein the at least one additional foaming agent is selected from the group consisting of mono- & diglycerides of fatty acids, esters of monoglycerides of fatty acids, mono- & diglycerides, propylene glycol monoesters, lecithin, hydroxylated lecithin, dioctyl sodium sulphosuccinate, sodium stearoyl-2-lactylate (SSL), calcium stearoyl lactate (CSL), sorbitan monolaurate (Polysorbate 20), sorbitan monopalmitate (Polysorbate 40), sorbitan monostearate (Polysorbate 60), sorbitan monooleate (Polysorbate 80), sorbitan tristearate, stearyl citrate, polyglycerol polyricinoleate (PGPR), lactylates, sodium lauryl ether sulfate (SLES), sodium dodecyl sulfate (SDS), ammonium lauryl sulfate (ALS), cocamide diethanolamine, triethanolamine, sodium lauroyl sarcosinate (INCl), and combinations thereof.

6. The foaming agent of claim 4, wherein the foaming agent comprises between about 25% to about 99.9% by weight of soy whey protein.

7. The foaming agent of claim 1, wherein the molecular weight of the soy whey protein is between about 8 kDa and about 50 kDa.

8. A personal care product comprising the foaming agent of claim 1.

9. The personal care product of claim 8, wherein the product is selected from the group consisting of toothpaste, hair color product, face soap, hand soap, body soap, and hair styling foam.

10. The personal care product of claim 8, wherein the foaming agent is present in the industrial product in an amount from about 0.1% to about 25% by weight of the product.

11. The personal care product of claim 10, wherein the foaming agent is present in the personal care product in an amount from about 1% to about 10% by weight of the product.

12. An industrial product comprising the emulsifying agent of claim 1.

13. The industrial product of claim 12, wherein the product is selected from the group consisting of foam mattress, foam cushion, foam pillow, dish soap, laundry soap, a fire retardant product, and a packaging product.

14. The industrial product of claim 12, wherein the foaming agent is present in the industrial product in an amount from about 0.1% to about 25% by weight of the product.

15. A product comprising a foaming agent, wherein the foaming agent comprises an amount of soy whey protein and wherein the product is a personal care product or an industrial product.

16. The product of claim 15, wherein the product further comprises water.

17. The product of claim 15, wherein the product further comprises at least one additional foaming agent.

18. The product of claim 17, wherein the at least one additional foaming agent is selected from the group consisting of mono- & diglycerides of fatty acids, esters of monoglycerides of fatty acids, mono- & diglycerides, propylene glycol monoesters, lecithin, hydroxylated lecithin, dioctyl sodium sulphosuccinate, sodium stearoyl-2-lactylate (SSL), calcium stearoyl lactate (CSL), sorbitan monolaurate (Polysorbate 20), sorbitan monopalmitate (Polysorbate 40), sorbitan monostearate (Polysorbate 60), sorbitan monooleate (Polysorbate 80), sorbitan tristearate, stearyl citrate, polyglycerol polyricinoleate (PGPR), lactylates, sodium lauryl ether sulfate (SLES), sodium dodecyl sulfate (SDS), ammonium lauryl sulfate (ALS), cocamide diethanolamine, triethanolamine, sodium lauroyl sarcosinate (INCl), and combinations thereof.

19. The product of claim 15, wherein the product further comprises an ingredient selected from the group consisting of thickening agent, fragrance, pigment, preservatives, and combinations thereof.

20. A method of making a product comprising a foaming agent, the method comprising the steps of:
(1) combining the foaming agent with water to form an aerated mixture; and
(2) processing the aerated mixture into the product, wherein the foaming agent comprises an amount of soy whey protein and wherein the product is a personal care product or an industrial product.

21. The method of claim 20, wherein the foaming agent further comprises at least one additional foaming agent.

22. The method of claim 20, further comprising adding to the aerated mixture an additional ingredient selected from the group consisting of a thickening agent, fragrance, pigment, preservatives, and combination thereof.

23. The method of claim 20, wherein the foaming agent is present in the industrial product in an amount of from about 0.1% to about 25% by weight.

24. The method of claim 20, wherein the personal care product is selected from the group consisting of toothpaste, hair color product, face soap, hand soap, body soap, hair styling foam,

25. The method of claim 20, wherein the industrial product is selected from the group consisting of foam insulation, foam mattress, foam rubber, foam packaging, dish soap, and laundry soap.

26. A stabilized foaming agent comprising an amount of soy whey protein.

27. The stabilized foaming agent of claim 26, wherein the soy whey protein has a soluble solids index (SSI) of at least about 80% across a pH range of from 2 to 10 and a temperature of 25° C.

28. The stabilized foaming agent of claim 26, wherein the molecular weight of the soy whey protein is between about 8 kDa and about 50 kDa.

29. A product comprising the stabilized foaming agent of claim 26, wherein the product is a personal care product or an industrial product.
